(19) United States
(12) Patent Application Publication
(10) Pub. No.: US 2010/0199548 A1
(43) Pub. Date: Aug. 12, 2010

(54) SYSTEMS AND METHODS FOR THE PRODUCTION OF FATTY ESTERS

(75) Inventors: Stephen del Cardayre, So. San Francisco, CA (US); Michael Charles Milner Cockrem, Madison, WI (US)

Correspondence Address: LS9, Inc., 600 Gateway Boulevard, South San Francisco, CA, 94080, US

(73) Assignee: LS9, Inc., So. San Francisco, CA (US)

(21) Appl. No.: 12/278,964

(22) PCT Filed: Jul. 2, 2008
(86) PCT No.: PCT/US08/69075

§ 371 Date: Apr. 19, 2010

Related U.S. Application Data
(60) Provisional application No. 60/948,406, filed on Jul. 6, 2007.
(60) Provisional application No. 61/054,427, filed on May 19, 2008.

(51) Int. Cl. C10L 1/19; C12P 7/64; C12M 1/00; C12N 1/15; C12N 1/21; C12N 1/16; C12N 1/18

(52) U.S. Cl. 44/388; 435/134; 435/289.1; 435/254.2; 435/254.21; 435/252.3; 435/252.31; 435/252.35; 435/252.34; 435/254.22; 435/254.23; 435/252.33

(57) ABSTRACT

Disclosed herein are various embodiments regarding the use of impure and/or unrefined alcohol in the production of fatty esters. Various production hosts that are capable of producing a fatty ester from an impure or unrefined alcohol are also disclosed.

### REFERENCE TO RELATED APPLICATIONS

The present application claims priority to U.S. provisional Application Nos. 60/948,406, filed Jul. 6, 2007 and 61/054,427, filed May 19, 2008, both of which are incorporated by reference in their entireties.

### FIELD OF THE INVENTION

The present disclosure relates generally to systems and methods for producing fatty esters and the fatty ester products made by the systems and methods.

### BACKGROUND

Developments in technology have been accompanied by an increased reliance on fuel sources and such fuel sources are becoming increasingly limited and difficult to acquire.

As a result, efforts have been directed toward harnessing sources of renewable energy, such as sunlight, water, wind, and biomass. The use of biomasses to produce new sources of fuel which are not derived from petroleum sources, (e.g., biofuel, such as biodiesel) has emerged as one alternative option. Current methods of making biodiesel involve transesterification of triacylglycerides (e.g., vegetable oil) which leads to a mixture of fatty esters and glycerin.

As demand for biofuels grow, there is a continuing need for new biofuels and for methods and systems of economically producing the biofuels.

### SUMMARY OF THE INVENTION

The present disclosure provides systems and methods for producing fatty esters, which can be utilized as a biofuel (e.g., biodiesel). Advantageously, in some embodiments, the systems and methods allow for the production of fatty esters from unrefined or relatively impure alcohol sources, which can provide a more economical starting material for fatty ester production than refined or purer forms of alcohol.

In some embodiments, the present disclosure provides a method of making a fatty ester. The method comprises processing an alcohol production substrate to produce an alcohol composition that comprises an alcohol, providing the alcohol composition, without refining the alcohol composition, to a fatty ester production host, providing a fatty ester production substrate to the fatty ester production host, and processing the fatty ester production substrate in the presence of the alcohol to produce a fatty ester.

In some embodiments, the present disclosure provides a method of making a fatty ester. The method comprises providing an alcohol composition to a fatty ester production host. The alcohol composition contains less than about 20% alcohol by volume immediately prior to being provided to the fatty ester production host. The method further comprises adding a fatty ester production substrate to the fatty ester production host and processing the fatty ester production substrate in the presence of the alcohol composition to produce a fatty ester.

In some embodiments, the present disclosure provides a production system. The production system can comprise a fatty ester production vessel, a fatty ester production host, and a source of impure alcohol in fluid communication with the fatty ester production vessel.

In some embodiments, the present disclosure provides a production system. The production system can comprise a production substrate storage unit and an ethanol production vessel comprising an ethanol production host. The ethanol production vessel can be in fluid communication with the production substrate storage unit. The production system can further comprise a fatty ester production vessel comprising a fatty ester production host. The fatty ester production vessel is in fluid communication with the production substrate storage unit and the fatty ester production host comprises a heterologous nucleic acid sequences encoding a thioesterase, an ester synthase, and an acyl-CoA synthase.

In some embodiments, the present disclosure provides a fatty ester composition. In some embodiments, the fatty ester composition comprises an alcohol production host and a fatty ester. In some embodiments the fatty ester is at least 40% by volume of the fatty ester composition.

In some embodiments, the present disclosure provides a biofuel. In some embodiments, the present disclosure provides a fatty ester composition comprising a fatty ester, wherein the fatty ester is produced according to any of the methods disclosed herein.

In other embodiments, the present disclosure provides a biofuel comprising a fatty ester, wherein the fatty ester is produced according to any of the methods disclosed herein.

In one embodiment, the present disclosure provides a production system having a fatty ester production subsystem. The system also includes a source of a substantially unrefined ethanol in communication with the fatty ester production subsystem. In particular configurations, the fatty ester production subsystem produces fatty esters from the substantially unrefined ethanol, such as an ethanol stream which includes between about 1% and about 25% by volume ethanol. In some examples, the system includes a production substrate storage unit in communication with the fatty ester production subsystem. The production substrate of the production substrate storage unit, in more particular examples, is a production substrate which includes glucose to be fermented by a production host in the fatty ester production subsystem. In particular implementations, the fatty acid production host includes a microorganism adapted to produce fatty acids or derivatives thereof and having one or more exogenous nucleic acid sequences encoding at least one thioesterase (EC 3.1.2.14), and at least one of a wax synthase (EC 2.3.1.75), an alcohol acetyltransferase (2.3.1.84), fatty alcohol forming acyl-CoA reductase (1.1.1.*), and the combination of at least one acyl-CoA reductase (EC 1.2.1.50) and at least one alcohol dehydrogenase (EC 1.1.1.1).

In another embodiment, the present disclosure provides a production system having an ethanol production subsystem and a fatty ester production subsystem, which are operated in series in some examples and in parallel in other examples. The ethanol production subsystem produces a product that includes ethanol, such as a stream of unrefined ethanol, which is provided as a feed to the fatty acid production system. The fatty acid production system includes a production host adapted to produce a fatty acid or fatty acid derivative, such as from a glucose containing production substrate. In particular examples, the fatty acid production host includes a microorganism having one or more exogenous nucleic acid sequences encoding at least one thioesterase (EC 3.1.2.14), and at least one of a wax synthase (EC 2.3.1.75), an alcohol acetyltransferase (2.3.1.84), fatty alcohol forming acyl-CoA reductase (1.1.1.*), and the combination of at least one acyl-CoA reductase (EC 1.2.1.50) and at least one alcohol dehydrogenase (EC 1.1.1.1).

In particular implementations, the production system includes a production substrate storage unit which provides a feed to the production system, such as to the ethanol production subsystem. In more particular examples, the production substrate storage unit also provides a feed to the fatty ester production subsystem. In specific examples of this system the production substrate storage unit is the only source of production substrate for the system.

Where the disclosed systems include ethanol and fatty ester production subsystems, the subsystems include ethanol and fatty acid or fatty acid derivative production vessels, in some examples. In more particular examples, the ethanol and fatty acid production vessels are in fluid communication. Other embodiments of the present disclosure provide a system where ethanol fermentation and fatty acid or fatty acid derivative fermentation are carried out in a common production vessel.

In various examples of embodiments of the present disclosure, the production system can be a closed system for producing fatty esters in which the only externally supplied production substrate is a carbohydrate, such as glucose, xylose, and other fermentable carbon sources, including sources derived from starch or lignocellulosic biomass. The production substrate is glucose in some examples. In such a system the fermentable carbon source is transformed into ethanol, and the ethanol is supplied to a fatty ester fermentation sub-system.

The present disclosure also provides methods of producing fatty esters, such as for use as biofuels. In a particular method, a fermentable carbon source stream is fermented to produce ethanol, such as unrefined ethanol. The ethanol is provided to a fatty acid or fatty acid derivative production host to produce a fatty ester. In particular configurations, a glucose feed stream is also provided to the fatty acid or fatty acid derivative production host. In some examples, the production host includes a microorganism having one or more exogenous nucleic acid sequences encoding at least one thioesterase (EC 3.1.2.14), and at least one of a wax synthase (EC 2.3.1.75), an alcohol acetyltransferase (2.3.1.84), fatty alcohol forming acyl-CoA reductase (1.1.1.*), and the combination of at least one acyl-CoA reductase (EC 1.2.1.50) and at least one alcohol dehydrogenase (EC 1.1.1.1).

There are additional features and advantages of the subject matter described herein. They will become apparent as this specification proceeds.

In this regard, it is to be understood that this is a brief summary of varying aspects of the subject matter described herein. The various features described in this section and below for various embodiments can be used in combination or separately. Any particular embodiment need not provide all features noted above, nor solve all problems or address all issues in the prior art noted above.

### BRIEF DESCRIPTION OF THE DRAWINGS

Certain embodiments will be described in more detail with reference to the following drawings:

FIG. 1 illustrates an embodiment of a production system for producing fatty esters according to the present disclosure.

FIG. 2 shows the FAS biosynthetic pathway.

FIG. 3 shows biosynthetic pathways that produce fatty esters.

FIG. 4 shows biosynthetic pathways that produce fatty alcohols.

FIG. 5 shows biosynthetic pathways that produce fatty esters.

FIGS. 6A-6D show GS-MS spectra of octyl octanoate (C8C8) produced by E. coli expressing alcohol acetyl transferase (AATs, EC 2.3.1.84) and ester synthase (EC 2.3.1.75).

FIG. 6A shows ethyl acetate extract of strain E. coli C41(DE3, ΔfadE/pCOLADuet-1-atfA1/pRSET B/pCDFDuet-1-fadD-acr1) wherein the plasmid expressed atfA (ester synthase).

FIG. 6B shows ethyl acetate extract of strain E. coli C41(DE3, ΔfadE/pCOLADuet-1-atfA/pRSET B/pCDFDuet-1-fadD-acr1) wherein the pCOLADuet-1-atfA plasmid expressed saat.

FIG. 6C shows ethyl acetate extract of strain E. coli C41(DE3, ΔfadE/pCOLADuet-1-atfA/pRSET B/pCDFDuet-1-fadD-acr1) wherein the pCOLADuet-1-atfA plasmid did not contain atfA (ester synthase) or saat.

FIG. 6D shows the mass spectrum and fragmentation pattern of C8C8 produced by C41(DE3, ΔfadE/pCOLADuet-1-atfA/pRSET B/pCDFDuet-1-fadD-acr1) wherein the pCOLADuet-1-atfA plasmid expressed SAAT).

FIG. 7 shows the distribution of ethyl esters made when the ester synthase from A. baylyi ADP1 (AtfA) was co-expressed with thioesterase gene from Cuphea hookeriana in a production host.

FIGS. 8A and 8B show chromatograms of GC/MS analysis.

FIG. 8A shows a chromatogram of the ethyl acetate extract of the culture of E. coli C41(DE3, ΔfadE) strain transformed with plasmids pCDFDuet-1-fadD-atfA and pETDuet-1-'tesA. Ethanol was fed to the E. coli.

FIG. 8B shows a chromatogram of ethyl hexadecanoate and ethyl oleate used as reference.

FIG. 9 shows a table that identifies examples of various genes that can be over-expressed or attenuated to increase fatty acid derivative production in various embodiments.

FIGS. 10A, 10B, 10C, and 10D show the results of a GC/MS analysis.

FIG. 10A shows a bar chart summarizing the fatty esters present in the supernatant and the fatty esters present in the pellet obtained from production runs using distilled ethanol or unrefined ethanol (e.g., beer), (using plasmid pCDFDuet-1-fadD-atfA (including ester synthase atfA and FadD) transformed along with plasmid pMAL-c2X-TEchfatB3 (thioesterase ChfatB3) into C41 (DE3, ΔfadE) cells).

FIG. 10B shows a bar chart summarizing the total fatty esters present in both the supernatants and pellets of FIG. 10A.

FIG. 10C shows a bar chart summarizing the total fatty esters for three separate distilled ethanol runs (control 1-3) and using either the vector alone or the unrefined ethanol.

FIG. 10D shows a bar chart summarizing the total fatty esters for the unrefined ethanol at various time points.

### DETAILED DESCRIPTION OF SOME PREFERRED EMBODIMENTS

Fatty esters can be made using various fatty ester production hosts. The production hosts are typically fed refined, highly concentrated, or highly pure alcohol compositions. Processes to refine or increase the purity of alcohol compositions can add undesirable costs and complexities to fatty ester production systems and methods.

Advantageously, the present disclosure provides systems and methods that do not require high purity, high concentration, and/or refined alcohol. As such, in some embodiments, the invention involves the use of impure (and/or unrefined) alcohol compositions in the production of fatty esters using a fatty ester production host. In some embodiments, when the correct production host is used, one can use an impure alcohol composition to synthesize a fatty ester. In some embodiments, this allows one to take an alcohol composition directly from its source and to use that alcohol as a substrate for a fatty ester production host to produce a fatty ester. In some embodiments, the alcohol is not refined prior to its use in the fatty ester production.

The following section presents the meaning of various terms and abbreviations. It also provides various alternative embodiments. Following this section is a general description of various embodiments, which is followed by a section outlining additional specific variations of the various embodiments and parts thereof. This section is then followed by a series of examples that outline various specific embodiments.

### ABBREVIATIONS, TERMS, VARIOUS EMBODIMENTS

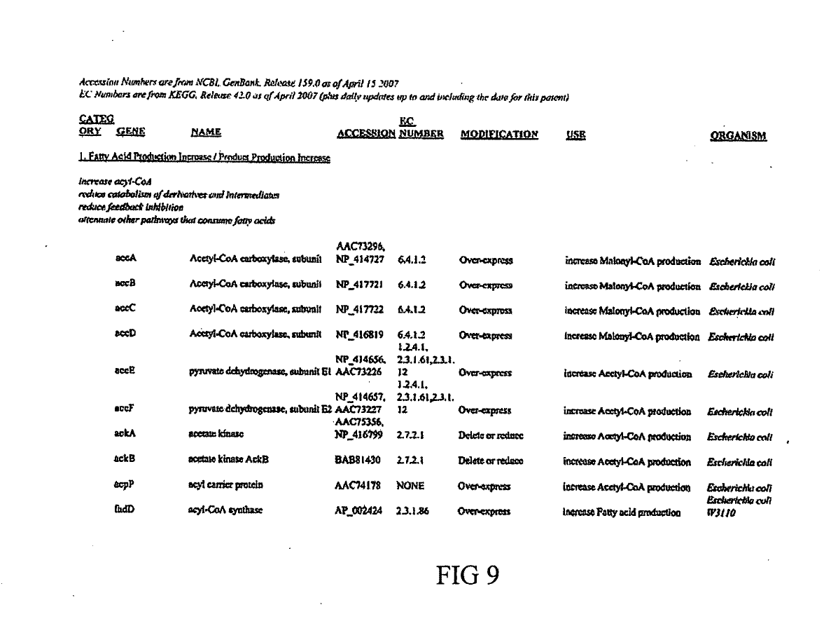

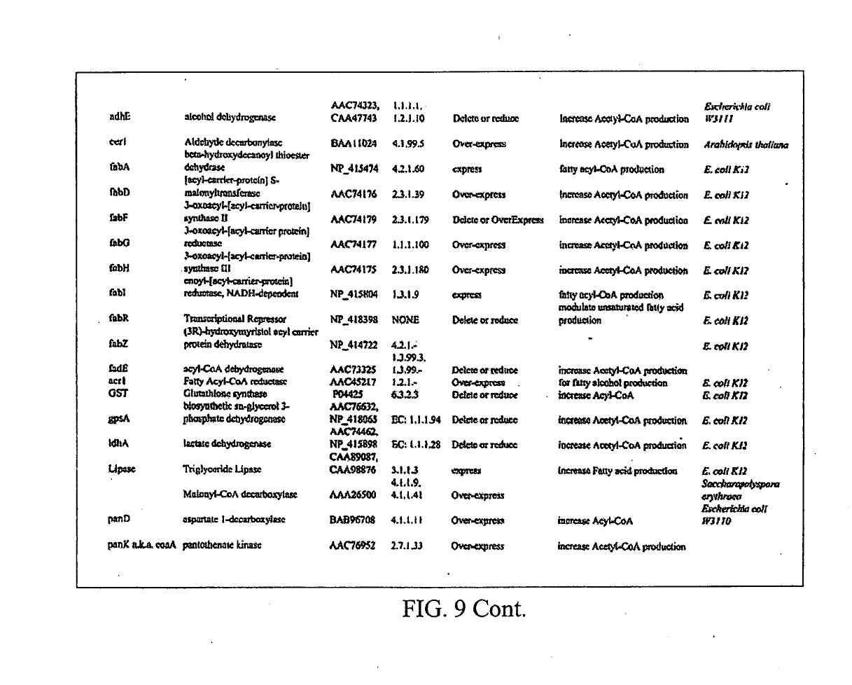

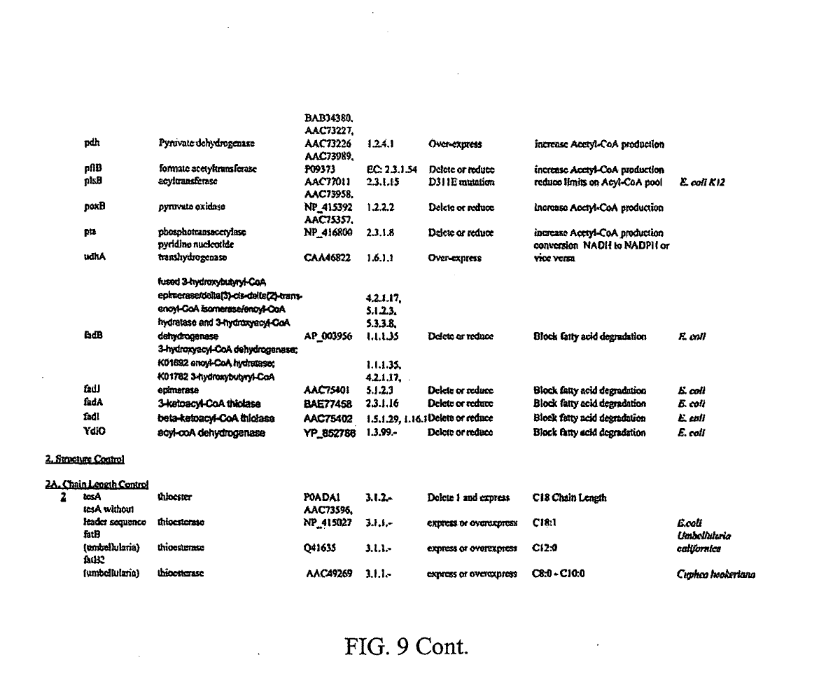

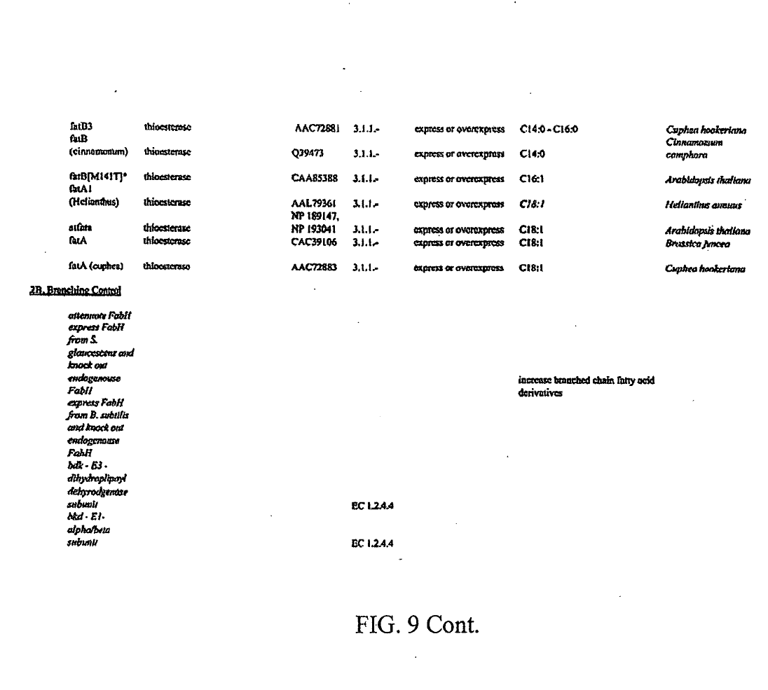

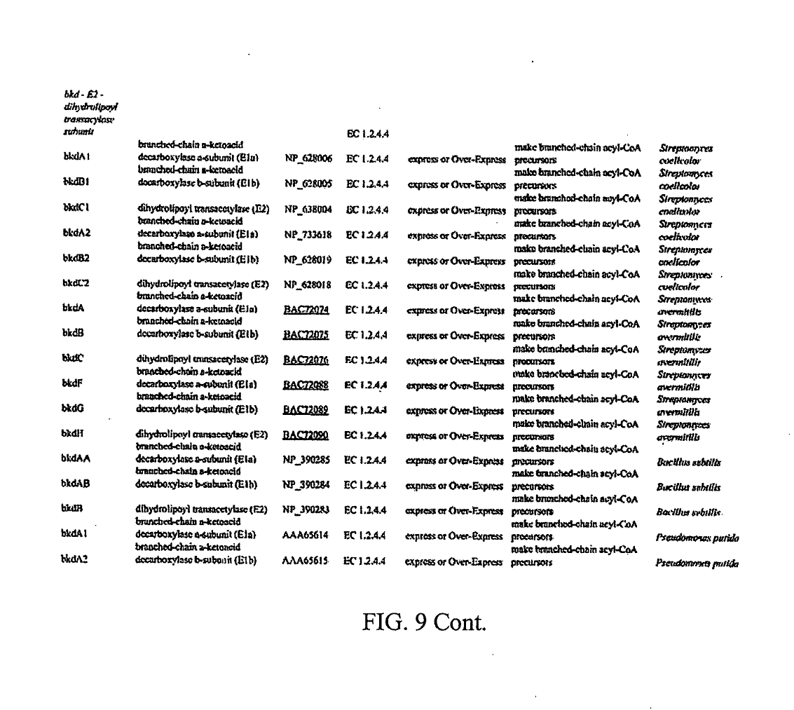

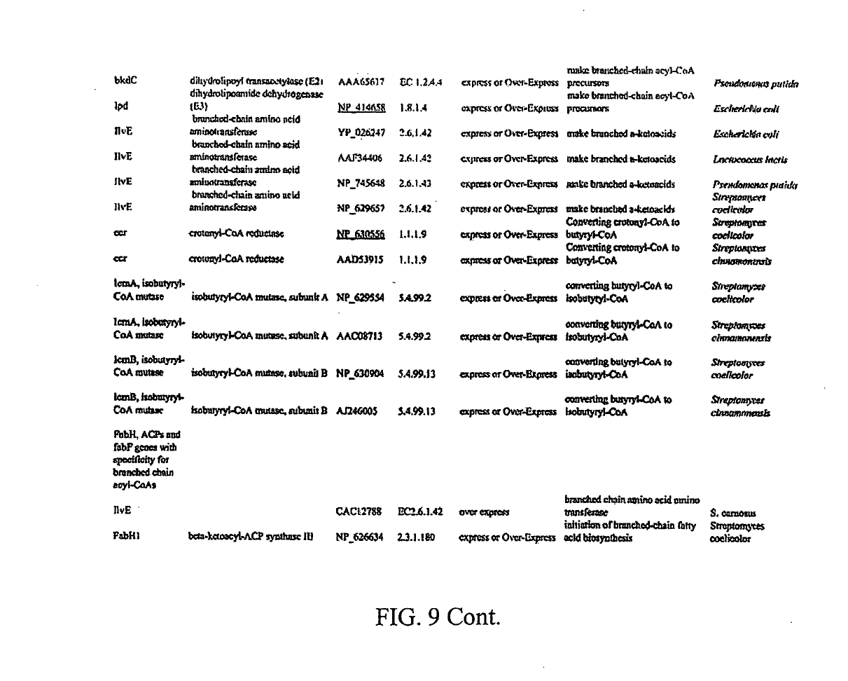

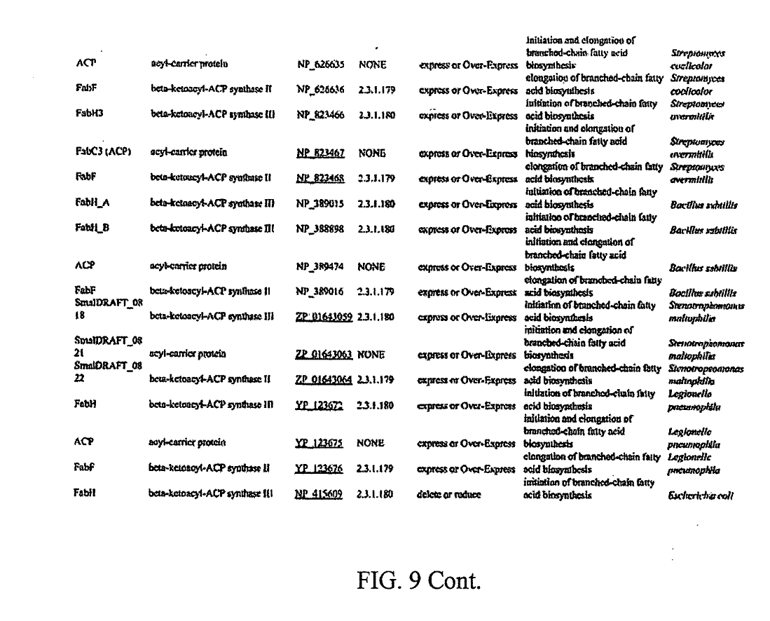

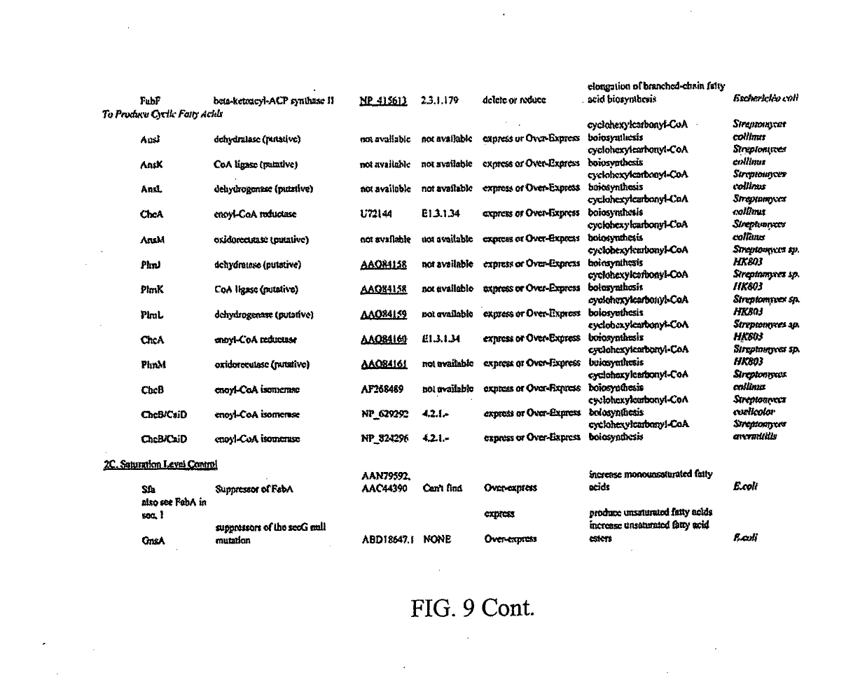

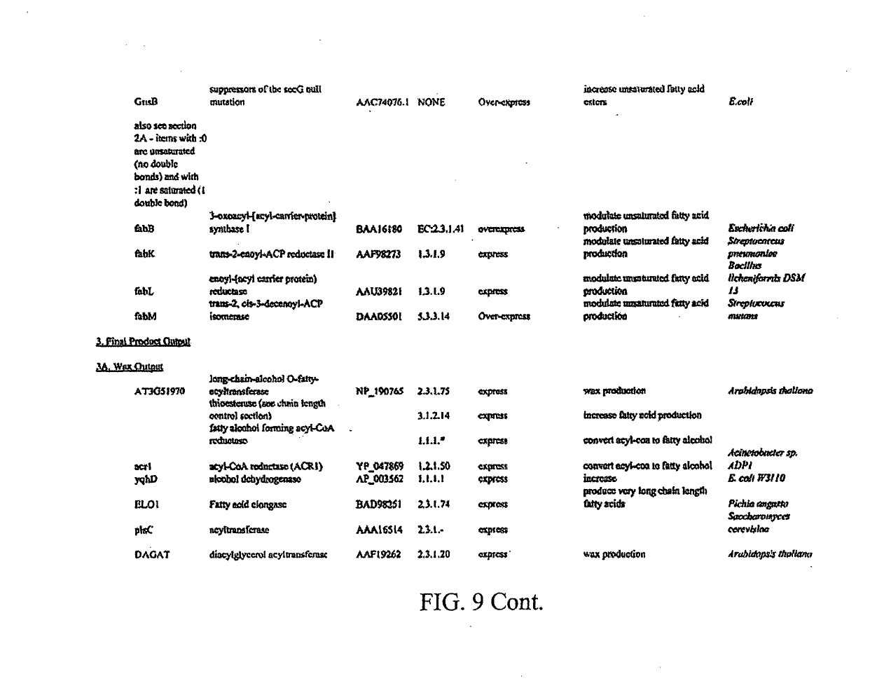

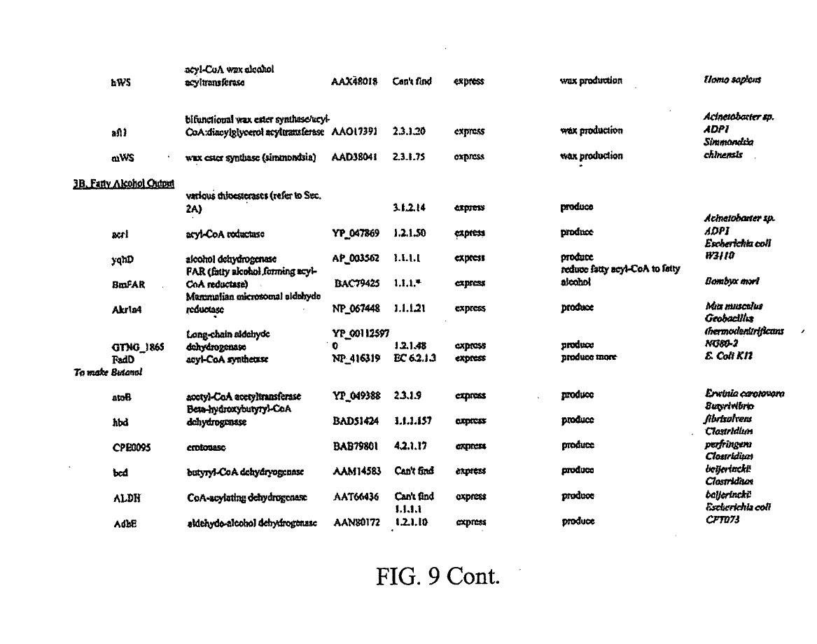

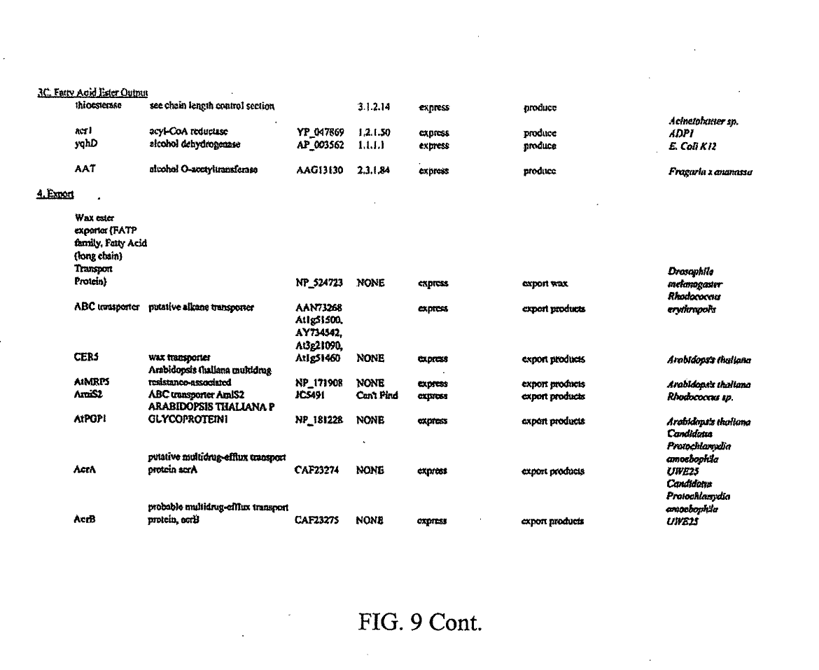

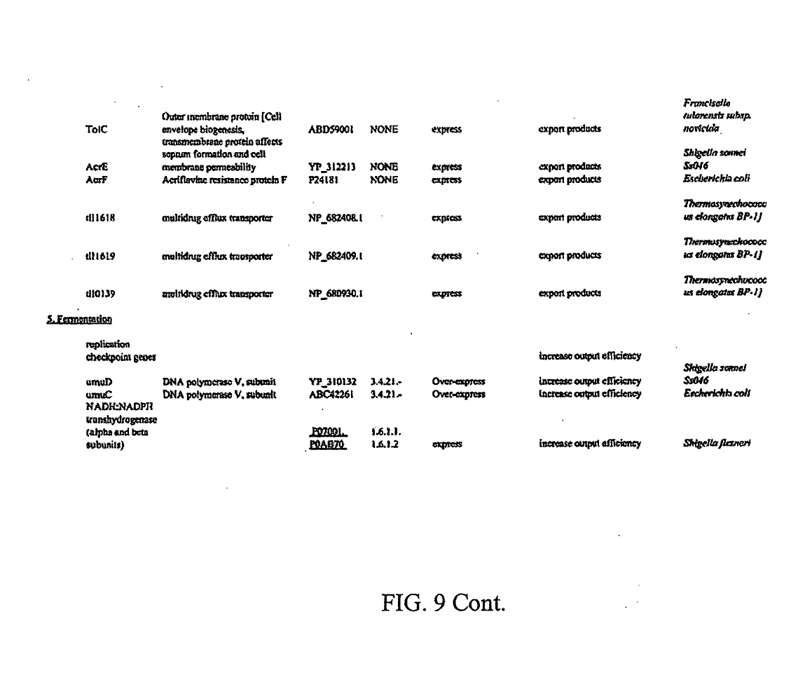

The following explanations of terms and methods are provided to better describe features of the present disclosure and to guide those of ordinary skill in the art in the practice of the present disclosure. As used herein, the singular forms “a,” “an,” or “the” include plural references unless the context clearly dictates otherwise. For example, reference to “a cell” or “the cell” includes one or a plurality of such cells. The term “or” refers to a single element of stated alternative elements or a combination of two or more elements, unless the context clearly indicates otherwise. For example, the phrase “thioesterase activity or fatty alcohol-forming acyl-CoA reductase activity” refers to thioesterase activity, fatty alcohol forming acyl-CoA reductase activity, or a combination of both thioesterase activity and fatty alcohol forming acyl-CoA reductase activity. Additionally, throughout the specification, a reference may be made using an abbreviated gene name or enzyme name, but it is understood that such an abbreviated gene or enzyme name represents the genus of genes or enzymes. For example “fadD” refers to a gene encoding the enzyme “FadD,” as well as genes encoding acyl-CoA synthase (EC 6.2.1.-). Such gene names include all genes encoding the same peptide and homologous enzymes having the same physiological function. Enzyme names include all peptides that catalyze the same fundamental chemical reaction or have the same activity. FIG. 9 provides various abbreviated gene and peptide names, descriptions of their activities, and their enzyme classification numbers. These can be used to identify other members of the class of enzymes having the associated activity and their associated genes, which can be used to produce fatty acid derivatives.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and are not intended to be limiting. Other features of the disclosure are apparent from the following detailed description and the claims.

Accession Numbers: The accession numbers throughout this description are derived from the NCBI database (National Center for Biotechnology Information) maintained by the National Institute of Health, U.S.A. The accession numbers are as provided in the database on Mar. 27, 2007.

Alcohol Composition: Denotes a composition comprising an alcohol molecule and at least one nonalcohol molecule. For example, a mixture comprising ethanol and water would be an alcohol composition. A mixture comprising alcohol and benzene would be another example of an alcohol composition. In some embodiments, at least 0.0001% of the composition is an alcohol (by volume). In some embodiments, such as when alcohol is being produced in the same vessel as the fatty ester, there is no lower requirement for the amount of alcohol that needs to be present in an alcohol composition.

Enzyme Classification Numbers (EC): EC numbers are established by the Nomenclature Committee of the International Union of Biochemistry and Molecular Biology (NC-IUBMB) (available at http://www.chem.qmul.ac.uk/iubmb/enzyme/). The EC numbers provided herein are derived from the KEGG Ligand database, maintained by the Kyoto Encyclopedia of Genes and Genomics, sponsored in part by the University of Tokyo. The EC numbers are as provided in the database on Mar. 27, 2007.

Attenuate: To weaken, reduce or diminish. For example, a polypeptide can be attenuated by modifying the polypeptide to reduce its activity (e.g., by modifying a nucleotide sequence that encodes the polypeptide). In another example, an enzyme that has been modified to be less active can be referred to as attenuated. In some embodiments, a gene or protein that has been removed or deleted can be characterized as having been attenuated.

Biofuel: The term “biofuel” refers to any fuel derived from biomass.

Biomass is a biological material that can be converted into a biofuel. One exemplary source of biomass is plant matter. For example, corn, sugar cane, and switchgrass can be used as biomass. Another non-limiting example of biomass is animal matter, for example cow manure. Biomass also includes waste products from industry, agriculture, forestry, and households. Examples of such waste products which can be used as biomass are fermentation waste, straw, lumber, sewage, garbage and food leftovers. Biomass also includes sources of carbon, such as carbohydrates (e.g., sugars).

In some embodiments, biofuels can be substituted for petroleum based fuels. For example, biofuels are inclusive of transportation fuels (e.g., gasoline, diesel, jet fuel, etc.), heating fuels, and electricity-generating fuels. Biofuels are a renewable energy source. Non-limiting examples of biofuels are biodiesel, hydrocarbons (e.g., alkanes, alkenes, alkynes, or aromatic hydrocarbons), and alcohols derived from biomass.

Biodiesel: Biodiesel is a form of biofuel. Biodiesel can be a substitute of diesel, which is derived from petroleum. Biodiesel can be used in internal combustion diesel engines in either a pure form, which is referred to as “neat” biodiesel, or as a mixture in any concentration with petroleum-based diesel.

Biodiesel can be comprised of hydrocarbons or esters. In some embodiments, biodiesel is comprised of fatty esters, such as fatty acid methyl esters (FAME) or fatty acid ethyl esters (FAEE). In some embodiments, these FAME and FAEE are comprised of fatty acyl moieties having a carbon chain length of about 8-20, 10-18, or 12-16 carbons in length. Fatty esters used as biodiesel may contain carbon chains which are saturated or unsaturated.

Carbon Source: Generally refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, gases (e.g., CO and CO₂), etc. These include, for example, various monosaccharides, such as glucose, fructose, mannose, and galactose; oligosaccharides, such as fructo-oligosaccharide and galacto-oligosaccharide; polysaccharides, such as xylose and arabinose; disaccharides, such as sucrose, maltose, and turanose; cellulosic material, such as methyl cellulose and sodium carboxymethyl cellulose; saturated or unsaturated fatty esters, such as succinate, lactate, and acetate; alcohols, such as ethanol, etc., or mixtures thereof.

The carbon source can additionally be a product of photosynthesis, including, but not limited to glucose. The carbon source can additionally be a carbon containing gas, such as carbon dioxide, carbon monoxide, or syngas.

cDNA (complementary DNA): A piece of DNA lacking internal, non-coding segments (introns) and regulatory sequences which determine transcription.

Cloud Point of a Fluid: The temperature at which dissolved solids are no longer completely soluble, precipitating as a second phase giving the fluid a cloudy appearance. This term is relevant to several applications with different consequences.

In the petroleum industry, cloud point refers to the temperature below which wax or other heavy hydrocarbons crystallizes in a crude oil, refined oil or fuel to form a cloudy appearance. The presence of solidified waxes influences the flowing behavior of the fluid, the tendency to clog fuel filters/injectors etc., the accumulation of wax on cold surfaces (e.g., pipeline or heat exchanger fouling), and even the emulsion characteristics with water. Cloud point is an indication of the tendency of the oil to plug filters or small orifices at cold operating temperatures.

The cloud point of a nonionic surfactant or glycol solution is the temperature where the mixture starts to phase separate and two phases appear, thus becoming cloudy. This behavior is characteristic of non-ionic surfactants containing polyoxyethylene chains, which exhibit reverse solubility versus temperature behavior in water and therefore “cloud out” at some point as the temperature is raised. Glycols demonstrating this behavior are known as “cloud-point glycols” and are used as shale inhibitors. The cloud point is affected by salinity, being generally lower in more saline fluids.

Cloud Point Lowering Additive: An additive which may be added to a composition to decrease or lower the cloud point of a solution, as described above.

Deletion: The removal of one or more nucleotides from a nucleic acid molecule or one or more amino acids from a protein, the regions on either side being joined together. A deletion can also refer to the missing nucleotide(s) from the nucleic acid molecule.

Detectable: Capable of having an existence or presence ascertained. For example, production of a product from a reactant (e.g., the production of C18 fatty acids) is detectable using the methods provided below.

Endogenous: As used herein, with reference to a nucleic acid molecule and a particular cell or microorganism, “endogenous” refers to a nucleic acid sequence or peptide that is in the cell and was not introduced into the cell using recombinant engineering techniques. For example, a gene that was present in the cell when the cell was originally isolated from nature. A gene is still considered endogenous if the control sequences, such as a promoter or enhancer sequences that activate transcription or translation, have been altered through recombinant techniques.

In some embodiments, if an endogenous sequence is cloned into a different location in the genome of its native cell, or is introduced into the cell as a component of a plasmid, then the gene would no longer be endogenous, but exogenous.

Ester Synthase: An ester synthase is a peptide capable of producing fatty esters. More specifically, an ester synthase is a peptide which converts a thioester to a fatty ester. In a preferred embodiment, the ester synthase converts the thioester, acyl-CoA, to a fatty ester.

In an alternate embodiment, an ester synthase uses a thioester and an alcohol as substrates to produce a fatty ester. Ester synthases are capable of using short and long chain acyl-CoAs as substrates. In addition, ester synthases are capable of using short and long chain alcohols as substrates.

Non-limiting examples of ester synthases are wax synthases, wax-ester synthases, acyl-CoA:alcohol transacylases, acyltransferases, and fatty acyl-coenzyme A:fatty alcohol acyltransferases. Exemplary ester synthases are classified in enzyme classification number EC 2.3.1.75. Exemplary GenBank Accession Numbers are provided in FIG. 9.

Exogenous: As used herein, with reference to a nucleic acid molecule and a particular cell, “exogenous” refers to any nucleic acid molecule that does not originate from that particular cell as found in nature. For example, “exogenous DNA” could refer to a DNA sequence that was inserted within the genomic DNA sequence of a microorganism, or an extra chromosomal nucleic acid sequence that was introduced into the microorganism. Thus, a non-naturally-occurring nucleic acid molecule is considered to be exogenous to a cell once introduced into the cell. A nucleic acid molecule that is naturally-occurring can also be exogenous to a particular cell. For example, an entire coding sequence isolated from an E. coli DH5 alpha cell is an exogenous nucleic acid with respect to a second E. coli DH5 alpha cell once that coding sequence is introduced into the second E. coli DH5 alpha cell, even though both cells are DH5 alpha cells.

Expression: The process by which the inheritable information in a gene, such as the DNA sequence, is made into a functional gene product, such as protein or RNA.

Several steps in the gene expression process may be modulated, including the transcription step, the translational step, and the post-translational modification of the resulting protein. Gene regulation gives the cell control over its structure and function, and it is the basis for cellular differentiation, morphogenesis, and the versatility and adaptability of any organism. Gene regulation may also serve as a substrate for evolutionary change, since control of the timing, location, and amount of gene expression can have a profound effect on the functions (actions) of the gene in the organism.

Expressed genes include genes that are transcribed into messenger RNA (mRNA) and then translated into protein, as well as genes that are transcribed into types of RNA, such as transfer RNA (tRNA), ribosomal RNA (rRNA), and regulatory RNA that are not translated into protein.

Fatty Ester: A fatty ester is an ester. In a preferred embodiment, a fatty ester is any ester made from a fatty acid, for example a fatty acid ester.

In some embodiments, a fatty ester is described as having an A side (i.e., the carbon chain attached to the carboxylate oxygen) and a B side (i.e., the carbon chain comprising the parent carboxylate). In some embodiments, when the fatty ester is derived from the fatty acid biosynthetic pathway, the A side is contributed by an alcohol, and the B side is contributed by a fatty acid.

Any alcohol can be used to form the A side of the fatty esters. For example, the alcohol can be derived from the fatty acid biosynthetic pathway. Alternatively, the alcohol can be produced through non-fatty acid biosynthetic pathways. For example, the alcohol can be produced by the terpenoid pathway or through the branched chain amino acid synthesis or degradation pathways. Moreover, the alcohol can be provided exogenously. For example, the alcohol can be supplied in the production broth in instances where the fatty ester is produced by an organism. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. The B side of the ester is at least about 4, 6, 8, 10, 12, 14, 16, 18, 20, 22, 24, or 26 carbons in length. The A side and/or the B side can be straight or branched chain. The branched chains may have one or more points of branching. In addition, the branched chains can include cyclic branches. Furthermore, the A side and/or B side can be saturated or unsaturated. If unsaturated, the A side and/or B side can have one or more points of unsaturation. As used herein, the B side can include linear alkanes, branched alkanes, and cyclic alkanes (e.g., cycloalkanes).

In some embodiments, the fatty ester is described as follows:

B₁COOA₁

Where B₁ (also known as the B side) is an aliphatic carbon group, such as an alkyl group. In some embodiments, B₁ comprises, consists, or consists essentially of a chain of carbons at least 5, 6, 7, 8, 9, 10, 11, 12, 13, 14, 15, 16, 17, 18, 19, 20, 21, 22, 23, 24, 25, 26, 27, 28, 29, or 30 carbons in length. A₁ (also known as the A side) will include at least one carbon and can be an aliphatic group, such as an alkyl group. In some embodiments, the alkyl group comprises, consists or consists essentially of 1, 2, 3, 4, 5, 6, 7, 8, 9, 10, 11, 12, 13, 14, 15, 16, 17, 18, 19, or 20 carbon atoms.

In one embodiment, the fatty ester is produced biosynthetically. In this embodiment, first the fatty acid is “activated.” Non-limiting examples of “activated” fatty acids are acyl-CoA, acyl ACP, and acyl phosphate. Acyl-CoA can be a direct product of fatty acid biosynthesis or degradation. In addition, acyl-CoA can be synthesized from a free fatty acid, a CoA, and an adenosine nucleotide triphosphate (ATP). An example of an enzyme which produces acyl-CoA is acyl-CoA synthase

After the fatty acid is activated, it can be readily transferred to a recipient nucleophile. Exemplary nucleophiles are alcohols, thiols, amines, or phosphates.

In another embodiment, the fatty ester can be derived from a fatty acyl-thioester and an alcohol.

In one embodiment, the fatty ester is a wax. The wax can be derived from a long chain alcohol and a long chain fatty acid. In another embodiment, the fatty ester is derived from a long chain alcohol and acetyl-CoA. For example, the long chain alcohol could be derived from fatty acid biosynthesis or from terpenoid biosynthesis. The resulting esters include alkyl acetates, isopentenyl acetate, geranyl acetate, farnesyl acetate, and geranyl acetate. In another embodiment, the fatty ester is a fatty acid thioester, for example fatty acyl Coenzyme A (CoA). In other embodiments, the fatty ester is a fatty acyl panthothenate, an acyl carrier protein (ACP), or a fatty phosphate ester.

Fatty esters have many uses. For examples, fatty esters can be used as a biofuel, a surfactant, or as the intermediate to the synthesis of a commodity, specialty, or fine chemicals, such as fuels, alcohols, olefins, and pharmaceuticals.

Fatty Acid Derivative: The term “fatty acid derivative” includes products made in part from the fatty acid biosynthetic pathway of the production host organism. “Fatty acid derivative” also includes products made in part from acyl-ACP or acyl-ACP derivatives. The fatty acid biosynthetic pathway includes fatty acid synthase enzymes which can be engineered as described herein to produce fatty acid derivatives, and in some examples can be expressed with additional enzymes to produce fatty acid derivatives having desired structural characteristics. Exemplary fatty acid derivatives include, for example, short and long chain alcohols, hydrocarbons, fatty alcohols, and esters, including waxes or fatty esters.

Fatty Acid Derivative Enzymes: All enzymes that may be expressed or overexpressed that affect the production of fatty acid derivatives are collectively referred to herein as fatty acid derivative enzymes. These enzymes may be part of the fatty acid biosynthetic pathway. Non-limiting examples of fatty acid derivative synthases include fatty acid synthases, thioesterases, acyl-CoA synthases, acyl-CoA reductases, alcohol dehydrogenases, alcohol acyltransferases, acetyl-CoA, acetyl transferases, fatty alcohol-forming acyl-CoA reductase, and ester synthases. Fatty acid derivative enzymes convert a substrate into a fatty acid derivative. In some examples, the substrate may be a fatty acid derivative which the fatty acid derivative enzyme converts into a different fatty acid derivative. Additional exemplary fatty acid derivative enzymes include enzymes such as those in glycolysis, acetyl-CoA carboxylase, and panK.

Fatty Alcohol Forming Peptides: Peptides capable of catalyzing the conversion of acyl-CoA to fatty alcohol, including fatty alcohol forming acyl-CoA reductase (FAR, EC 1.1.1.*), acyl-CoA reductase (EC 1.2.1.50) or alcohol dehydrogenase (EC 1.1.1.1). Additionally, one of ordinary skill in the art will appreciate that some fatty alcohol forming peptides will catalyze other reactions as well. For example, some acyl-CoA reductase peptides will accept other substrates in addition to fatty acyl-CoA. Such non-specific peptides are, therefore, also included. Nucleic acid sequences encoding fatty alcohol forming peptides are known in the art and such peptides are publicly available. Exemplary GenBank Accession Numbers are provided in FIG. 9.

Fraction of Modern Carbon: Fraction of modern carbon (f_{M}) is defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

Fermentation: Fermentation denotes the use of a carbon source by a production host. Fermentation can be aerobic, anaerobic, or variations thereof (such as micro-aerobic).

Functional Deletion: A mutation, partial or complete deletion, insertion, or other variation made to a gene sequence which reduces or inhibits production of the gene product, or renders the gene product non-functional. For example, functional deletion of fabR in E. coli reduces the repression of the fatty acid biosynthetic pathway and allows E. coli to produce more unsaturated fatty acids (uFAs). In some instances a functional deletion is described as a knock-out mutation.

In some embodiments, isolated refers to a naturally-occurring nucleic acid molecule that is not immediately contiguous with both of the sequences with which it is immediately contiguous (one on the 5′ end and one on the 3′ end) in the naturally-occurring genome of the organism from which it is derived.

Heterologous: Heterologous nucleic acid sequence denotes that the nucleic acid sequence has been genetically modified and/or is non-naturally occurring sequence. A sequence can be heterologous, even if the gene has been passed from one organism to another organism. Thus, bacteria produced from an initial bacterium with a heterologous gene would also contain a nucleic acid that is heterologous. Furthermore, differences by deletion or attenuation will also make an altered nucleic acid sequence heterologous.

Isolated: An “isolated” biological component (such as a nucleic acid molecule, protein, or cell) is a biological component that has been substantially separated or purified away from other biological components in which the biological component naturally occurs, such as other chromosomal and extra-chromosomal DNA sequences; chromosomal and extra-chromosomal RNA; and proteins. Nucleic acid molecules and proteins that have been “isolated” include nucleic acid molecules and proteins purified by standard purification methods. The term embraces nucleic acid molecules and proteins prepared by recombinant expression in a production host cell as well as chemically synthesized nucleic acid molecules and proteins.

In one example, isolated refers to a naturally-occurring nucleic acid molecule that is not contiguous with both of the sequences with which it is directly adjacent to (i.e., the sequence on the 5′ end and the sequence on the 3′ end) in the naturally-occurring genome of the organism from which it is derived.

Microorganism: Includes prokaryotic and eukaryotic microbial species from the domains Archaea, Bacteria and Eucarya, the latter including yeast and filamentous fungi, protozoa, algae, or higher Protista. The terms “microbial cells” and “microbes” are used interchangeably with the term microorganism.

Nucleic Acid Molecule: Encompasses both RNA and DNA sequences including, without limitation, cDNA, genomic DNA sequences, and mRNA. The term includes synthetic nucleic acid molecules, such as those that are chemically synthesized or recombinantly produced. The nucleic acid molecule can be double-stranded or single-stranded. When single-stranded, the nucleic acid molecule can be the sense strand or the antisense strand. In addition, a nucleic acid molecule can be circular or linear.

Operably Linked: A first nucleic acid sequence is operably linked to a second nucleic acid sequence when the first nucleic acid sequence is placed in a functional relationship to the second nucleic acid sequence. For instance, a promoter is operably linked to a coding sequence if the promoter is in a position to affect the transcription or expression of the coding sequence. Generally, operably linked DNA sequences are contiguous and may join two protein coding regions, in the same reading frame. Configurations of separate genes which are operably linked and are transcribed in tandem as a single messenger RNA are denoted as operons. Placing genes in close proximity, for example in a plasmid vector, under the transcriptional regulation of a single promoter, constitutes a synthetic operon.

ORF (open reading frame): A series of nucleotide triplets (i.e., codons) coding for amino acids without any termination codons. These sequences are usually translatable into a peptide.

Over-express: When a peptide is present in a greater concentration in a recombinant host cell compared to its concentration in a non-recombinant host cell of the same species. Over-expression can be accomplished using any method known in the art. For example, over-expression can be caused by altering the control sequences in the genomic DNA sequence of a host cell, introducing one or more coding sequences into the genomic DNA sequence, altering one or more genes involved in the regulation of gene expression (e.g., deleting a repressor gene or producing an active activator), amplifying the gene at a chromosomal location (tandem repeats), introducing an extra chromosomal nucleic acid sequence, increasing the stability of the RNA transcribed via introduction of stabilizing sequences, and combinations thereof.

Examples of recombinant microorganisms that over-produce a peptide include microorganisms that express nucleic acid sequences encoding acyl-CoA synthases (EC 6.2.1.-). Other examples include microorganisms that have had exogenous promoter sequences introduced upstream to the endogenous coding sequence of a thioesterase peptide (EC 3.1.2.-). Over-expression also includes elevated rates of translation of a gene compared to the endogenous translation rate for that gene. Methods of testing for over-expression are well known in the art. For example, transcribed RNA levels can be assessed using rtPCR and protein levels can be assessed using SDS page gel analysis.

Partition Coefficient: The partition coefficient, P, is defined as the equilibrium concentration of a compound in an organic phase divided by the concentration at equilibrium in an aqueous phase (e.g., production broth). In one embodiment of the bi-phasic system described herein, the organic phase is formed by the fatty acid derivative during the production process. However, in some examples, an organic phase can be provided, such as by providing a layer of octane, to facilitate product separation. When describing a two phase system, the partition coefficient, P, is usually discussed in terms of log P. A compound with a log P of 1 would partition 10:1 to the organic phase. A compound with a log P of −1 would partition 1:10 to the organic phase. By choosing an appropriate production broth and organic phase, a fatty acid derivative with a high log P value will separate into the organic phase even at very low concentrations in the production vessel.

Process or Production: The term “process” or “production,” when used in reference to a production host denotes the biological manipulation of a production substrate via a production host to result in a product.

Production Broth: Includes any production medium which supports microorganism life (i.e., a microorganism that is actively metabolizing carbon). When noted, a production broth also can refer to “spent” production broth, a production broth which no longer supports microorganism life, and production broths with diminished capacity to support such life, such as being depleted or partially depleted of a carbon source, such as glucose.

Production Host: A production host is a cell that can produce one or more of the products disclosed herein. As disclosed herein, the production host can be modified to express or over-express selected genes, or to have attenuated expression of selected genes. Non-limiting examples of production hosts include plant, animal, human, bacteria, yeast, or filamentous fungi cells. There are various species of production hosts and are generally named by the product they produce. Thus, a fatty ester production host will at least produce fatty esters, an alcohol production host will at least produce an alcohol, and an ethanol production host will at least produce ethanol.

As noted herein, the production hosts can often have heterologous nucleic acid sequences or lack certain otherwise endogenous nucleic acid sequences.

Production Medium: As used herein, can refer to the medium in which a production process occurs. In some embodiments, the production medium can include a production host, a production substrate, and other substances, such as nutrients for the production host, process additives, carriers, or solvents.

Nutrients which can be included in some production media include buffers, minerals, and growth factors. Growth factors can include vitamins, such as biotin, thiamine, pantothenate, nicotinic acid, riboflavin, meso-inositol, folic acid, para-aminobenzoic acid, vitamins A, B (including niacin), C, D, and E, and pyridoxine. Additional growth factors which can be included are peptides or amino acids, such as tryptophan, glutamine, and asparagine. Enzymes can also be included as nutrients or process additives, such as to assist in production, such as by conversion of a substrate to a form more easily fermented by the production host or assisting in the conversion of a substrate to a production product, such as ethanol or a fatty ester.

Minerals which can be included in the production medium include Mg, P, K, Ca, Cu, S, Zn, Fe, Co, Mn, Ni, and Mo and ions, or other inorganic substances, such as ammonium, phosphate, sulfate, chloride, sodium, and borate. Nitrogen sources can also be included in the production media, such as ammonia, urea, ammonium nitrate, ammonium sulfate, grain meal.

Suitable production media are described in Jayme et al., Culture Media for Propagation of Mammalian Cells, Viruses, and Other Biologicals, Advances in Biotechnical Processes 5, p. 1 (1985). Examples of suitable production media include lysogeny broth, corn steep liquor (CSL), M9 minimal medium, SOC medium, Terrific broth, SOB medium, NZM medium, NZCYM medium, MZYM medium, and ZXYT medium.

The chemical and physical properties of the production medium can also be adjusted to suit the needs of a particular production process, production host, or production substrate. For example, in yeast to produce ethanol, the pH of the production medium is typically between about pH 4.0 and about pH 8.8, such as between about pH 4.0 and about pH 5.0. In some examples to produce fatty esters, the pH of the production medium is between about pH 6.0 and about pH 8.0, such as between about pH 6.5 and about pH 7.5 or between about pH 7.0 and about pH 7.4.

In particular examples of yeast fermentation to produce ethanol or when using fatty ester production hosts according to the present disclosure, the temperature of the production medium is maintained at about 10° C. to about 47° C., such as about 30° C. to about 45° C. or about 20° C. to about 40° C. The temperature of the fermentation can be adjusted to produce a desired production rate, for the needs of a particular production host, or can be chosen to facilitate the overall production process. The production temperature can be adjusted during the course of a production, such as being maintained at a higher temperature initially and then decreasing the temperature once production is underway or reaches a certain point, which can be indicated by a chance in the consumption of an input, such as oxygen, or production of an output, such as carbon dioxide. For example, a fatty ester production process can be held at a first temperature for a first part of the production and a second, lower, temperature for a second part of the production, such as after the addition of ethanol to the production.

Production Substrate: Refers to one or more materials which serve as a source of carbon for a production host during a production process (e.g., production of an alcohol or a fatty ester). Different production substrates can be used for different production processes. This will depend on the production host, the production process, and the desired product. For example, when ethanol is the desired product, suitable production substrates include, for example, a carbon source, such as a carbohydrate (e.g., sugar, starch, lignocellulosic biomass, or cellulose), carbon monoxide, or syngas. When fatty esters are the desired product, suitable production substrates include carbon sources, such as, carbohydrates (e.g., glucose), starch, cellulose, lignocellulosic biomass, carbon monoxide, syngas, or ethanol.

Suitable carbohydrate containing substrates for ethanol and fatty ester production include, for example, biological sources, such as sugarcane, sweet sorghum, or sugar beets. Suitable starch sources include, for example, cassava, millet, tapioca, wheat, barley, corn, rice, potatoes, rye, triticale, sorghum grain, sweet potatoes, and Jerusalem artichokes. In further embodiments, the ethanol and fatty esters are produced from biomass, such as grasses (e.g., energy cane, switchgrass, and mycanthus), legumes (e.g., soybeans and peas), algae, seaweed, bagasse, corn stover, pulp and paper mill residues, paper, corn fiber, agricultural residue, plant materials, and wood. In yet further embodiments, the production substrate is a municipal or industrial waste source, such as paper, waste sulfite liquors, or fruit or vegetable wastes from processing plants or canning operations.

In some cases, such as with production substrates having a high content of reducing sugar (e.g., sugar cane and sugar beets), the production substrate can be added to the production medium or production vessel without preprocessing or with minimal processing. For example, a solid production substrate can be broken down into smaller pieces to facilitate production or processing. In particular implementations, the production substrate is milled, either dry or wet, such as using a hammer mill. In further examples, the production substrate is passed through a dispersing machine, such as an in-line machine running the Supramyl process or a batch process using Ultra-Turrax dispersing machines (available from IKA Works, Inc., of Wilmington, N.C.).

However, other production substrates, such as starches or cellulose materials can be subjected to one or more processing steps in order to put the production substrate into a suitable form for production. For example, cellulose materials, such as lingocellulose materials, can be subjected to a hydrolysis, or saccharification, pretreatment step to convert the cellulose to more easily fermentable compounds, such as sugar, including reducing sugars, such as glucose. Hydrolysis, in some implementations, is acid hydrolysis. In other implementations enzymatic hydrolysis is used to convert the cellulose to a more easily fermentable form.

Acid hydrolysis can be carried out using dilute acid, such as 1% sulfuric acid, in a continuous flow reactor at relatively higher temperatures (such as about 215° C.) with a conversion ratio of about 50%. Concentrated acid hydrolysis can be carried out by treating the substrate with 70% sulfuric acid at about 100° F. for 2-6 hours to convert hemicellulose to sugar, followed by treatment with 30 to 40% sulfuric acid for 1 to 4 hours, followed by 70% sulfuric acid treatment for about 1 to about 4 hours. The conversion rate using concentrated acid is typically about 90%. Enzymatic hydrolysis can be carried out using a suitable cellulase enzyme, such as a cellulase derived from Trichoderma viride or Trichoderma reesei. In particular examples, hydrolysis and production are carried out in the same vessel, in a process referred to as Simultaneous Saccharification and Fermentation (SSF).

In particular examples, such as when the production substrate includes a starchy material, the production substrate can be liquefied prior to fermentation, such as by heating and the addition of enzymes, as described in paragraphs 68-71 of U.S. Patent Publication US2007/0082385. The starches can be converted to sugars using various starch reducing enzymes. In particular examples, enzymatic starch reduction is accomplished using as a combination of liquefying α-amylases and saccharifying glucoamylases. Suitable α-amylases include thermostable bacterial α-amylase of Bacillus licheniformis (TBA) (typically used in a production medium having a pH between about 6.2 to about 7.5 at a temperature of about 80° C. to about 85° C.), bacterial alpha-amylase of Bacillus subtilis (BAA) (typically used in a production medium having a pH between about 5.3 to about 6.4 and a temperature of about 50° C.); bacterial alpha-amylase expressed by Bacillus licheniformis (BAB) (typically used in a production medium having a pH between about 4.5 to about 4.8 and a temperature of about 90° C.); and fungal alpha-amylase of Aspergillus oryzae (typically used in a production medium having a pH between about 5.5 to about 8.5 and a temperature between about 35° C. and about 60° C.).

Saccharifying glucoamylases include beta-amylases (such as alpha-1,4-glucan maltohydrolase (EC 3.2.1.2)), and alpha-amylases; glucoamylase (EC 3.2.1.3). Glucoamylase of Aspergillus niger (GAA) (which can operate at a pH range of 3.4 to 5.0 e.g., 4.5 to 5.0; and at a temperature range of 55° C. to 70° C., 60° C.); Glucoamylase of Rhizopus sp. (GAR) (which can operate at a pH range of 4.0 to 6.3, e.g., 4.0 to 5.5; and at a temperature range of 40° C.-60° C. Combinations of glucoamylases can also be used, such as GAR and FAA or GAR, GAA, and FAA. Suitable starch reducing enzymes include those present in malted grains.

Grain malting can be accomplished using any suitable technique, many of which are well known in the art. Prior to mashing, a high pressure cooking process, such as in a jet cooker, can be used to release starches from the production substrate. In some examples, mashing is carried out in a stainless steel vessel, which can include a mechanical agitator. The temperature can be maintained at a desired temperature using heaters and cooling coils, such as stainless steel cooling coils. Heat exchangers can be used to conserve energy used in heating and cooling the mash, including spiral-plate, spiral-tubular, plate, or tubular heat exchangers. Suitable mashing processes include cold mashing, the Groβe-Lohmann-Spradau (GLS) process, and milling and mashing process at higher temperatures.

Other souces of a production substrate include carbon containing gases, such carbon monoxide and syngas. Carbon monoxide is a major waste stream from steal mills. When it is compressed it can be fed into a bioreactor as a source of reduced carbon. Syngas is a mixture gases including carbon monoxide, carbon dioxide, and hydrogen that can be generated from carbonaceous materials, such as coal and biomass. There are organisms, such as various Clostridial species, that can use carbon monoxide and/or syngas as a source of carbon and electrons to support growth and as a substrate for chemical production, such as for ethanol and polyhydroxyalkanoate production.

Production System: The various components, including at least a production vessel, used to produce a product, such as an alcohol, a fatty ester, and derivatives thereof, from a production substrate using a production host. The production system can include processes upstream from the production process itself or production vessel, such as substrate handling and conditioning processes. The production system can also include downstream processes, such as processes for separating the product from at least a portion of other components of a mixture from the production vessel. For example, separation can be accomplished by filtration, such as using a membrane filter, a string-discharge filter, or a knife discharge filter. Distillation can also be used to separate the product from at least a portion of the mixture from the production vessel.

In some implementations, the production system includes various components to aid or monitor the process. For example, in some configurations, the system includes defoamers, such as mechanical foam breakers (which, in some examples, are included in the production vessel) or chemical defoamers, such as fatty acids, polyglycols, higher alcohols, or silicones. Particular disclosed production systems include various monitors or sensors, including sensors to measure temperature, pH (such as glass and reference electrodes), dissolved oxygen, foam (such as conductance/capacitance probes), agitation speed (e.g., tachometer), air flow (e.g., rotameter, mass flow meter), pressure, fluid flow, CO₂ content, and specific gravity.

The production system can be run as a batch or continuous process, such as a continuous process with a cell cycle to return a portion of the production host to the production vessel, which can increase product yield. In some embodiments, the process is carried out under vacuum, such as a vacuum fermentation, which includes recycling of at least a portion of the production host. When vacuum fermentation is used, heat from the fermentation process can be used to distill at least a portion of the product, such as ethanol.

Steps can be taken to sterilize the production vessel or other components of the production system. In some methods, heat is used for sterilization, such as treating a surface with pressurized steam for a suitable period of time, for example applying steam at about 120° C. for about 20 minutes. Surfaces can also be disinfected chemically, such as using NaOH, nitric acid, sodium hypochlorite (bleach), ethylene oxide, peracetic acid, ozone, formaldehyde, or antibacterial agents, such as kanamycin, streptomycin, or carbenicillin. In some cases, surfactants are added to the disinfectant in order to help increase disinfectant permeation or penetration. In further implementations, filtration is used to help remove microbes from air or liquid streams. In particular examples, absolute filters having a pore opening of about 0.2 microns are used. In further embodiments, radiation, such as microwave or ultraviolet radiation, can be used to sanitize various system components, including feed or product streams.

Production Vessel: A vessel or container that holds a production host and a substrate, during at least a portion of a production process. Any suitable structure can be used as a production vessel, including those presently in laboratory and commercial use, such as tanks, vats, bags, bottles, flasks, or reactors. In particular implementations, the production vessel can be a stirred tank reactor equipped with a mechanical agitator. Suitable mechanical agitators include paddles, blades, impellers, propellers, or turbines. Tower reactors can also be used as production vessels, particular examples of which are described in U.S. Pat. Nos. 5,888,806 and 4,654,308; and Wieczorek et al., Continuous Ethanol Production by Flocculating Yeast in the Fluidized Bed Bioreactor, FEM Microbio. Rev., 4, pp. 69-74 (1994). In further implementations, the production vessel is a pneumatically agitated reactor, such as tower jet loop, plunging jet, tower jet, and tower pneumatic reactors. In some examples, pneumatic agitation can also serve to increase the oxygen level in the production medium for aerobic production.

In further embodiments, the production vessel is an immobilized microorganism bioreactor. In particular configurations, the production host is immobilized by adsorption onto a preformed carrier (such as wood chips, cellulose, glass, ceramic, or synthetic materials). In some examples, the production host is adsorbed only to the surface of the carrier, while in other examples the production host is also adhered in pores of the carrier. Another method of production host immobilization is by entrapment of the production host in a matrix, such as alginate, kappa-carrageenan, or pectate gels. The production host can also be immobilized by self-aggregation of cells, such as by cross-linking, or by containment of production host behind a barrier, such as encapsulating yeast cells within polyvinyl alcohol beads or plug flow reactors where the production host is retained by one or more support plates.

Various specific implementation of bioreactors using immobilized microorganisms include packed bed reactors, fluidized bed reactors, silicon carbide cartridge loops (silicone carbine rods seeded with yeast cells), or internal loop gas-lift reactors.

In particular embodiments where the production vessel is provided with gas, such as to agitate the vessel contents or to provide an oxygen source for production, the reactor vessel includes a gas inlet, such as a sparger for introducing the gas below the level of the production medium. Suitable gas inlets include one or more nozzles, nozzle clusters, rings or orifices, or porous materials, such as sintered metal or stone. The air source, in some implementations, is supplied by a compressor, such as a rotary, reciprocating, or centrifugal compressor. In some examples, the gas is filtered before introduction into the reactor vessel, such as using a membrane or activated carbon filter.

Promoters and Enhancers: Transcriptional control signals in eukaryotes comprise “promoter” and “enhancer” elements. Promoters and enhancers consist of short arrays of DNA sequences which interact specifically with cellular proteins involved in transcription (Maniatis et al., Science 236:1237, 1987). Promoter and enhancer elements have been isolated from a variety of eukaryotic sources including genes in yeast, insect, mammalian and plant cells. Promoter and enhancer elements can be isolated from viruses. Analogous control elements, such as promoters and enhancers, are also found in prokaryotes. The selection of a particular promoter and enhancer depends on the cell type used to express the protein of interest. Some eukaryotic and prokaryotic promoters and enhancers have a broad production host cell range while others are functional in a limited subset of production host cells (see, e.g., Voss et al., Trends Biochem. Sci., 11:287, 1986; and Maniatis et al., 1987 supra).

The terms “promoter element,” “promoter,” or “promoter sequence” refer to a DNA sequence that functions as a switch which activates the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Purified: The term “purified” refers to molecules that are removed from their natural environment by, for example, isolation or separation. “Substantially purified” molecules are at least about 60% free, preferably at least about 75% free, and more preferably at least about 90% free from other components with which they are naturally associated. As used herein, the term “purified” or “to purify” also refers to the removal of contaminants from a sample. For example, the removal of contaminants can result in an increase in the percentage of fatty acid derivatives of interest in a sample. For example, after fatty acid derivatives are expressed in plant, bacterial, yeast, or mammalian production host cells, the fatty acid derivatives are purified by the removal of production host cell proteins. After purification, the percentage of fatty acid derivatives in the sample is increased.

The term purified does not require absolute purity; rather, it is intended as a relative term. Thus, for example, a purified fatty acid derivative preparation is one in which the product is more concentrated than the product is in its environment within a cell. For example, a purified fatty ester is one that is substantially separated from cellular components (e.g., nucleic acids, lipids, carbohydrates, and other peptides) that can accompany it. In another example, a purified fatty ester preparation is one in which the fatty ester is substantially free from contaminants, such as those that might be present following production and/or fermentation.

For example, a fatty ester is purified when at least about 50% by weight of a sample is composed of the fatty ester. In another example when at least about 60%, 70%, 80%, 85%, 90%, 92%, 95%, 98%, or 99% or more by weight of a sample is composed of the fatty ester.

Recombinant: A recombinant nucleic acid molecule is one that has a sequence that is not naturally occurring, has a sequence that is made by an artificial combination of two otherwise separated segments of sequence, or both. This artificial combination can be achieved, for example, by chemical synthesis or by the artificial manipulation of isolated segments of nucleic acid molecules, such as genetic engineering techniques. Recombinant is also used to describe nucleic acid molecules that have been artificially manipulated, but contain the same regulatory sequences and coding regions that are found in the organism from which the nucleic acid was isolated. A recombinant protein is a protein derived from a recombinant nucleic acid molecule.

A recombinant or transformed cell is one into which a recombinant nucleic acid molecule has been introduced, such as an acyl-CoA synthase encoding nucleic acid molecule, for example by molecular biology techniques. Transformation encompasses all techniques by which a nucleic acid molecule can be introduced into such a cell, including, but not limited to, transfection with viral vectors, conjugation, transformation with plasmid vectors, and introduction of naked DNA sequence by electroporation, lipofection, and particle gun acceleration.

Release: The movement of a compound out of a cell. The movement can be active or passive. When release is active it can be facilitated by one or more transporter peptides and in some examples it can consume energy. When release is passive, it can be through diffusion through the membrane and can be facilitated by continually collecting the desired compound from the extracellular environment, thus promoting further diffusion. Release of a compound can also be accomplished by lysing a cell.

Substantially Impure Alcohol Composition: Refers to an alcohol composition that contains a significant amount of an impurity. While the amount of an impurity can vary, in some embodiments, the amount of impurity is at least a significant amount such as at least, or at least about 2, 2-5, 5-10, 10-15, 15-20, 20-25, 25-30, 30-40, 40-50, 50-60, 60-70, 70-80, 80-90, 90-99, or 99 percent of the alcohol mixture (by volume). In some embodiments, there can be more than one impurity. Impurities can include any compound that is not a fatty ester. In some embodiments, an impurity does not encompass water.

Substantially Unrefined Alcohol Composition: Refers to alcohol that has not been subjected to substantial purification and/or concentration steps. In some embodiments, the alcohol can be produced via a biological process, such as fermentation. Substantially unrefined alcohol typically includes about 0.1% to 30% ethanol by volume, for example about 0.1, 1, 2, 3, 4, 5, 6, 7, 8, 9, 10, 111, 12, 13, 14, 15, 16, 17, 18, 19, 20, 21, 22, 23, 24, 25, 26, 27, 28, 29, or 30 percent, or any value defined between any combination of any two of the preceding numbers. In particular implementations, unrefined alcohol includes alcohol at concentrations of less than 40% by volume. As will be appreciated by one of skill in the art, the term “unrefined alcohol” and “impure alcohol” implicitly denote an alcohol composition comprising at least alcohol and another substance. The remaining portion of unrefined alcohol can include solvents or carriers, such as water, unfermented sugars or other production substrates, solids, yeast or other production hosts, carbon dioxide, other organic compounds, including those produced by fermentation, proteins, and amino acids. Substantially unrefined alcohol does not exclude processes, such as filtration or separation of a portion of the components, such as by sedimentation or minor distillation. Substantially unrefined alcohol specifically excludes fully distilled or substantially fully distilled alcohol, including solutions greater than about 85% by volume alcohol, including anhydrous or absolute alcohol.

Surfactants: Substances capable of reducing the surface tension of a liquid in which they are dissolved. They are typically composed of a water-soluble head and a hydrocarbon chain or tail. The water soluble head is hydrophilic and can be either ionic or nonionic. The hydrocarbon chain is hydrophobic. Surfactants are used in a variety of products, including detergents and cleaners, and are also used as auxiliaries for textiles, leather, and paper, in chemical processes, in cosmetics and pharmaceuticals, in the food industry, and in agriculture. In addition, they can be used to aid in the extraction and isolation of crude oils which are found in hard to access environments or in water emulsions.

There are four types of surfactants characterized by varying uses. Anionic surfactants have detergent-like activity and are generally used for cleaning applications. Cationic surfactants contain long chain hydrocarbons and are often used to treat proteins. Amphoteric surfactants contain long chain hydrocarbons and are typically used in shampoos. Non-ionic surfactants are generally used in cleaning products.

Synthase: A synthase is an enzyme which catalyzes a synthesis process. As used herein, the term synthase includes synthases and synthetases.

Transformed or Recombinant Cell: A cell into which a nucleic acid molecule has been introduced. Transformation encompasses all techniques by which a nucleic acid molecule can be introduced into a cell, including, but not limited to, transfection with viral vectors, conjugation, transformation with plasmid vectors, and introduction of naked DNA by electroporation, lipofection, and particle gun acceleration.

Transport Protein: A protein that facilitates the movement of one or more compounds in and/or out of an organism or organelle. In some embodiments, an exogenous DNA sequence encoding an ATP-Binding Cassette (ABC) transport protein will be functionally expressed by the production host so that the production host exports the fatty acid derivative into the culture medium. ABC transport proteins are found in many organisms, such as Caenorhabditis elegans, Arabidopsis thalania, Alcaligenes eutrophus (later renamed Ralstonia eutropha), or Rhodococcus erythropolis. Non-limiting examples of ABC transport proteins include CER5, AtMRP5, AmiS2 and AtPGP1. In a preferred embodiment, the ABC transport protein is CER5 (e.g., AY734542).

In other embodiments, the transport protein is an efflux protein selected from: AcrAB, TolC, or AcrEF from E. coli or tll1618, tll1619, and tll0139 from Thermosynechococcus elongatus BP-1.

In further embodiments, the transport protein is a fatty acid transport protein (FATP) selected from Drosophila melanogaster, Caenorhabditis elegans, Mycobacterium tuberculosis, or Saccharomyces cerevisiae or any one of the mammalian FATPs well known in the art.

Under Conditions that Permit Product Production: Any production conditions that allow a production host to produce a desired product. Exemplary products include acyl-ACP, acyl-CoA and other fatty acid derivatives such as fatty acids, hydrocarbons, fatty alcohols, fatty esters, as well as, in some embodiments, alcohol(s). Production conditions usually comprise many parameters. Exemplary conditions include, but are not limited to, temperature ranges, levels of aeration, and media composition. Each of these conditions, individually and in combination, allows the production host to grow.

Exemplary mediums include liquids or gels. In some embodiments, the medium includes a carbon source, such as glucose, fructose, cellulose, or the like, that can be metabolized by the microorganism directly. In addition, enzymes can be used in the medium to facilitate the mobilization (e.g., the depolymerization of starch or cellulose to fermentable sugars) and subsequent metabolism of the carbon source.

To determine if the culture conditions permit product production, the production host can be cultured for a sufficient time (e.g., about 4, 8, 12, 24, 36, or 48 hours). During culturing or after culturing, samples can be obtained and analyzed to determine if the culture conditions permit product production. For example, the production hosts in the sample or the medium in which the production hosts were grown can be tested for the presence of the desired product. When testing for the presence of a product, assays, such as, but not limited to, TLC, HPLC, GC/FID, GC/MS, LC/MS, MS, as well as those provided in the examples below, can be used.

Vector: A nucleic acid molecule as introduced into a cell, thereby producing a transformed cell. A vector can include nucleic acid sequences that permit it to replicate in the cell, such as an origin of replication. A vector can also include one or more selectable marker genes or other genetic elements known in the art.

Wax: Wax is comprised of fatty esters. In a preferred embodiment, the fatty ester contains an A side and a B side comprised of medium to long carbon chains.

In addition to fatty esters, a wax may comprise other components. For example, wax can also comprise hydrocarbons, sterol esters, aliphatic aldehydes, alcohols, ketones, beta-diketones, triacylglycerols, etc.

### General Embodiments

As noted above, the present Inventors have appreciated that various fatty ester production hosts can not only use high purity or refined alcohol to produce fatty esters, but that these production hosts can also, surprisingly effectively, use impure, low concentration, and/or unrefined alcohol.

As will be appreciated by one of skill in the art, given the present disclosure, this ability can result in numerous advantageous results. For example, purification steps that previously were assumed to be important can be removed. As will be appreciated by one of skill in the art, this removes a time consuming step and the costs associate with that step. In addition, this aspect opens up additional sources of ethanol for use in fatty ester production. Furthermore, in some embodiments, the ability to use impure alcohol can be used to combine the alcohol production and fatty ester production steps to various extents.

Indeed, as noted below, in some embodiments the present invention allows one to make both the alcohol product and the fatty ester product in a single reaction vessel. This not only allows one to accelerate the process by removing numerous steps, but can also reduce the cost, space required, and general efficiency of using an alcohol to produce a fatty ester composition. These and additional embodiments and advantages are described in more detail below.

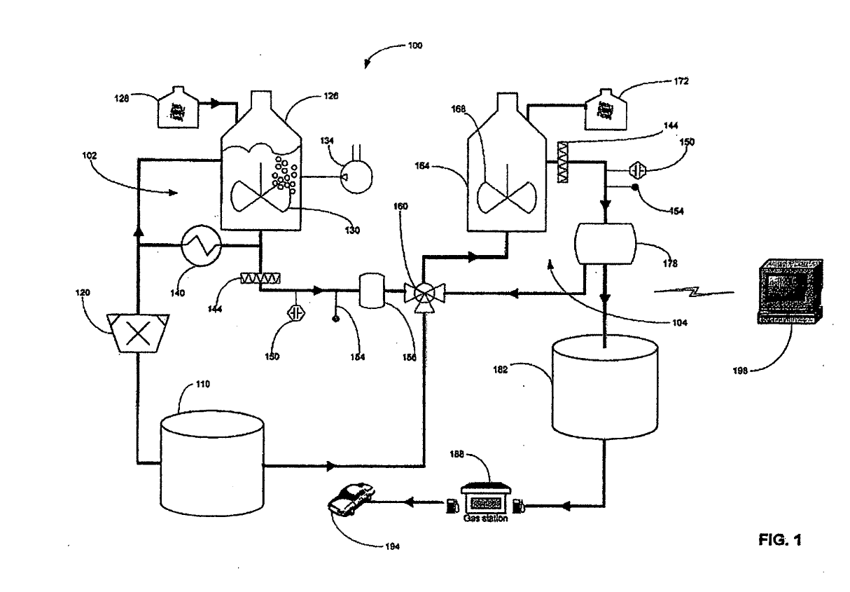

FIG. 1 displays an embodiment of a production system 100 for producing fatty acid derivatives, such as fatty esters. Fatty esters can be used as biodiesel. The system 100 includes a dual production process that includes an alcohol production subsystem 102 and a fatty ester production subsystem 104. Although illustrated with dual production subsystems 102 and 104, the system 100 can have other configurations. In various embodiments, the production subsystems 102 and 104 can be operated in series, parallel, or for cogeneration or coformation of their respective products. In some embodiments, both ethanol production and fatty ester production occur in a single subsystem or even a single production vessel.

In the embodiment depicted in FIG. 1, a production substrate storage unit 110 is in communication with one or more optional processes and the devices for performing the processes 120 upstream from an alcohol production vessel 126. Upstream processes and the devices for performing the processes 120 can include, for example, various pre-production treatment steps. Exemplary pre-production treatment step include, but are not limited to, mashing, saccharification, hydrolysis, milling, heating, dispersing, and/or liquefaction. In particular examples, the upstream processes are omitted or performed upstream from the storage unit 110.

The upstream processes and the devices for performing the processes 120 are in communication with the alcohol production vessel 126 in which the production media formed from the production substrate, and any additional components, is fermented into alcohol using a suitable production host. A production host seed vessel 128 is in communication with the alcohol production vessel 126 and can be used to deliver one or more production hosts to the alcohol production vessel 126. In further embodiments, the production host seed vessel 128 is omitted or is located upstream of the production vessel.

The alcohol production vessel 126 optionally includes a mechanical agitator 130, such as a propeller. In further embodiments, the mechanical agitator is omitted and, optionally, pneumatic or other means of agitation, such as baffles within the alcohol production vessel 126, is used.

In some embodiments, the alcohol production vessel 126 is also in communication with a gas source 134, such as a compressor delivering oxygen or air to the alcohol production vessel 126. The gas source 134 is used, in some embodiments, to provide oxygen to the alcohol production vessel 126 to assist in production. In further embodiments, the gas source 134 is used for pneumatic agitation of the alcohol production vessel 126. In yet further embodiments, the gas source 134 is omitted.

An outlet of the alcohol production vessel 126 is in communication with one or more downstream processes in the system of FIG. 1. In some embodiments, the downstream processes are omitted. In particular examples, the alcohol containing product stream from the alcohol production vessel 126 includes between about 0.5% and about 25% alcohol by volume, such as between about 5% and about 20% alcohol by volume. The concentration of alcohol in the product stream can be modified by various means, such as adjusting the concentration of production substrate or production host added to the alcohol production vessel 126, the production temperature, the production time, the nature of the production host, the nature of the production substrate, or the nature of the production medium. In certain implementations, production is carried for about 1 to about 100 hours, such as about 24 to about 96 hours.

As shown in FIG. 1, one optional downstream process in communication with the product stream of the alcohol production vessel 126 is a heat exchanger 140, which is also in communication with the input stream to the alcohol production vessel 126. The heat exchanger 140 can recycle energy in order to cool the product stream and heat the input stream. In further configurations, the heat exchanger 140 is omitted, located elsewhere in the system 100, or in communication with alternate or additional feeds.

Another optional downstream process is a filtration process (and device for performing a filtration) 144. In some examples, the filtration process 144 includes a screen or mesh filter, a membrane filter, a string-discharge filter, or a knife discharge filter. The downstream processes can also include various sensors 150, such as sensors to measure pH, dissolved oxygen, foam, turbulence, flow rate, CO₂ content, and specific gravity. The downstream processes also include a temperature sensor 154. In further embodiments, one or more of the filter 144, temperature sensor 154, or sensors 150 are omitted or placed in locations other than as shown in FIG. 1.

Another optional downstream processing step included in the system 100 is a distillation process 156, which can include an apparatus for performing the process. In further implementations, the distillation process 156 is located elsewhere in the system 100 or is omitted. The alcohol concentration in the product stream can also be concentrated by extracting a portion of the product stream. Alcohol, either from the system 100 or an external source, can also be added to the product stream in order to give a desired alcohol concentration. The alcohol concentration can also be diluted, such as by adding an aqueous solution to the product stream.

After passing through any downstream processes, the product stream from the alcohol production vessel 126 can be in communication with an optional mixing valve 160. The mixing valve 160 is also in communication with the substrate storage unit 110, a recycling feed from the fatty ester production system 104, and provides an input stream to the fatty ester production system 104.

The output stream from the optional mixing valve 160 is in communication with the fatty ester production vessel 164. The concentration of alcohol (e.g., ethanol) in the feed stream to the fatty ester production vessel 164 can be adjusted based on the contents of the fatty ester production vessel 164, such as the concentration of production substrate (e.g., sugar) to be used. In particular examples, the alcohol stream includes about 0.1 to about 25% by volume alcohol, such as about 4 to about 12% by volume. The amount of alcohol can be chosen so that it remains in excess, such as a 2-fold excess, compared with the concentration of fatty acyl-CoA produced by a production host from a production substrate (e.g., glucose). For example, 2 moles of alcohol can be provided for each mole of fatty acyl-CoA produced. In certain implementations, production is carried for about 1 to about 100 hours, such as about 24 to about 96 hours, at a temperature of about 15° C. to about 45° C., such as about 25° C. to about 35° C.

In some embodiments, the fatty ester production system 104 can be constructed generally as described with respect to the alcohol production system 102. As shown, the fatty ester production vessel 164 includes a mechanical agitator 168. However, in further embodiments, the mechanical agitator 168 is omitted or replaced with a pneumatic agitator or other sources of agitation, such as internal baffles. The fatty ester production vessel 164 is in communication with a fatty ester production seed vessel 172, which contains a suitable fatty ester production host, examples of which are provided by the present disclosure. In particular implementations, the fatty ester production seed vessel 172 is omitted or is located upstream of the production vessel 164.

A product stream of the production vessel 164 is optionally in communication with one or more downstream processes, such as filtration process 144, sensors 150, and temperature sensor 154, which can be configured as described with respect to the alcohol production system 102. The product stream of the production vessel 164 is also, optionally, in communication with a separation process 178, such as a filtration, distillation, or phase separation process, which can separate the fatty ester product from at least a portion of the product stream mixture. An output of the separation process 178 is optionally in communication with the mixing valve 160. Another optional output of the separation process is in communication with a product collection vessel 182. In further embodiments, one or more of the downstream processes 144, sensor 150, temperature sensor 154, and separation process 178 are located in a different order or omitted. For example, in a specific embodiment the separation process is omitted and the product of the production vessel 164 is extracted directly from the production vessel 164.

In some embodiments, the product collection vessel 182 is in communication with a product supply chain, such as a supply chain for supplying biodiesel, in the form of fatty esters, to distribution stations 188 for distribution to end users, such as for use in motor vehicles 194. In further embodiments, one or more of the product collection vessel 182, distribution station 188, or end users 194 are omitted.

The various components of the system 100 are in communication with one or more controllers, generally indicated as 198, which can be used to monitor the production system 100 or control various aspects of its operation.

In some embodiments, in operation of the system 100, a production substrate (e.g., an alcohol production substrate, fatty ester production substrate, or both) is provided from the substrate storage unit 110 and pretreated, such as by milling, in upstream processing 120, where it can also be fluidized, such as by adding the milled substrate to a liquid carrier, such as water. The substrate slurry is then transferred to the alcohol production vessel 126.

In some embodiments, the alcohol production host is added to the alcohol production vessel 126 from the feed vessel 128. The production medium is agitated in the alcohol production vessel 126 using the mechanical agitator 130 and aerated with air from the gas source 134. Heating and cooling components (not shown) are used, in some examples, to maintain the production medium at a desired temperature. The pH of the production medium can be adjusted through the addition of acids or bases.

In some embodiments, the product of the alcohol production system 102 is an unrefined alcohol solution containing additional substances, such as increased carbon dioxide, yeast, or other microorganisms, and unfermented production substrate (or any of the herein disclosed impurities). The amount of alcohol in the solution is typically about 0.1% to about 25% by volume, such as about 1% to about 25% or about 4% to about 12% by volume.

When the production has reached a desired degree of completion, the product stream from the alcohol production vessel 126 can, optionally, be passed through the heat exchanger 140, where it can be cooled. The cooled product stream can then, optionally, be filtered through filter 144 and various properties measured using sensors 150, 154. While not necessary, in some embodiments, the product stream is distilled in the distillation unit 156 to concentrate the alcohol in the product stream. In some examples, the distillation step is omitted.

After the distillation step, if included, the alcohol product stream optionally passes to the mixing valve 160 where it is mixed with a feed from the substrate storage unit 110. Although not shown in FIG. 1, the feed from the substrate storage unit 110 to the mixing valve 160 can first be subjected to one or more pretreatment steps, such as the steps described in conjunction with upstream processes 120.

In some embodiments, the mixing valve 160 also mixes the alcohol product and substrate feed streams with a recycling stream from the separation process 178. In further embodiments, the recycling stream or feed streams are omitted. In yet further embodiments, the mixing valve 160 is omitted and the alcohol product from the alcohol production subsystem 102 is fed directly into the fatty ester production vessel 164 or fatty ester production subsystem 104. In some such embodiments, the system 100 can include a feed from a production substrate storage unit, such as the production substrate storage unit 110, to the fatty ester production vessel 164 or the fatty ester production subsystem 104. In further implementations, the system 100 does not include an additional production substrate feed or storage unit, and the production substrate for the fatty ester production vessel 164 or fatty ester production subsystem 104 comes only from the alcohol production subsystem 102.

In some embodiments, a fatty ester production feed stream is prepared in the production vessel 164 for a period of time before an alcohol is added to the production vessel 164, optionally with additional fatty ester production feed, which can be added in one or more batches or over a period of time. Similarly, the alcohol can be added in one or more batches or over a period of time.

In some embodiments, the fatty ester production feed stream enters the production vessel 164, where it is combined with a feed from the fatty ester production host seed vessel 172. The production medium can be mixed with the mechanical agitator 168.

In some embodiments, after the production has reached a desired degree of completion, the production mixture is transferred from the production vessel 164, filtered using filter 144, and monitored using sensors 150 and 154. The product stream then is optionally transferred to the separation process 178 where the fatty ester product is separated from other components, including unreacted production substrate and alcohol, which are sent in a recycling stream to the mixing valve 160.

In some embodiments, the separated fatty ester product, which can be used as biodiesel fuel in some implementations, is then transferred to the product storage tank 182, sent to distributing stations 188, and provided to end users, such as for use in motor vehicles 194. In some embodiments, the processes of the system 100 are monitored and controlled using the workstation 198.

Numerous changes can be made to the system 100 without departing from the scope of the present disclosure. For example, the alcohol production vessel 126 and the fatty ester production vessel 164 can be combined into a single unit. Rather than being set up for batch productions, one or both systems 102 and 104 can be set up for continuous production. Although productions 102 and 104 are shown as part of a combined, parallel process, they can be operably separated. For example, alcohol production could be carried out in a separate plant before being used as a feed to a fatty ester production system. Although systems 102 and 104 are shown having a common production substrate, separate storage units, or separate substrates, can be used for each system. For example, when the alcohol and fatty ester subsystems are operably separated, each subsystem can be provided with a separate production substrate storage unit or supply. In further configurations, the alcohol and fatty ester production subsystems use different production substrates and thus are provided with different production substrate storage units or supplies. In an alternate embodiment, the alcohol and fatty ester subsystems are operably separated, but each is provided with a production substrate from the same production substrate storage unit or supply.

In yet further embodiments, the fatty ester production system can include a fatty ester production unit or subsystem where fatty esters are produced by a fatty ester production host, such as those disclosed herein.

The product of the fatty ester production system 104 can be selected as desired through the use of an appropriate production host, as described herein. Examples of products which can be prepared from the fatty ester production system 104 using disclosed production hosts include a product which is at least about 50 to about 100 wt % C_{16:1} ethyl ester, at least about 50 to about 100 wt % C_{18:1} ethyl ester, at least about 50 to about 100 wt % C_{16:0} ethyl ester, and/or at least about 50 to about 100% C_{18:0} ethyl ester. In some embodiments, the product is at least about 50 to about 95 wt % C_{16:1} ethyl ester, at least about 50 to about 95 wt % C_{18:1} ethyl ester, at least about 50 to about 95 wt % C_{16:0} ethyl ester, and/or at least about 50 to about 95% C_{18:0} ethyl ester.

In some embodiments, the product comprises, consists, or consists essentially of C15:0, C16:1, C18:1, C14:0, C14:1, or any mixture of these.

The conditions under which the system 100 is operated can vary based on numerous parameters, such as the size (operational capacity) of the system 100, the production feeds and hosts used, whether the system is configured for batch or continuous processing, and the desired products. As an example, the following parameters are provided for a fatty ester production process. Of course, these parameters can vary as the process is scaled up or down or different components used.

| | |
|---|---|
| Production vessel Size | 2 L |
| Total initial glucose | 7.5 g in 1.5 L |
| Total glucose added during | 215 g in 0.5 L |
| production | |
| Glucose solution addition rate | 0.1 mL/min ≧ x ≧ 0.5 ml/min |
| Alcohol (such as ethanol) | 45 mL (at start of feed glucose |
| | addition) |
| | 45 mL (after 12 hours) |
| Production host | 100 mg/L |
| pH | 7.2 |
| Temperature | 37° C. (startup) |
| | 30° C. (during glucose/ethanol |
| | addition) |

In some embodiments, the above parameters are scaled up appropriately for 10, 10-100, 100-1000, 10³-10⁴, 10⁴-10⁵, 10⁵-10⁶, 10⁶-10⁷, or more liters.

When both the alcohol and fatty ester processing is to occur in a single vessel, various modifications can be made to further yield beneficial results. For example, in some embodiments, the two production hosts perform their specific roles in similar conditions and can use a single substrate. In some embodiments, while the two production hosts can operate under similar conditions (e.g., pH and temperature), they can use different production substrates. For example, by using a modified Zymomonas mobilis that utilizes 5 carbon sugars, but not 6 carbon sugars, in combination with one of the fatty ester production hosts disclosed herein that utilizes 6 carbon sugars, one can effectively control the activity of both hosts in a single vessel by simply controlling the amount of the 5 carbon or 6 carbon sugar that is provided. In addition, the health of both the E. coli and the Z. mobilis are ensured since they both grow at similar pH and temperature. For example, Zymomonas mobilis can be engineered to utilize pentoses (see, e.g., Zhang et al., (1995). Metabolic engineering of a pentose metabolism pathway in ethanologenic Zymomonas Science 267, 240-243) and can be modified so that it is deficient in one or more of the genes required for glucose metabolism, such as that encoded by glucokinase (Georg. A. Sprenger (1996), Carbohydrate metabolism in Zymomonas mobilis: A Catabolic Highway with Some Scenic Routes. FEMS Microbiology let. 145, 301-307.)

In some embodiments, the method of making a fatty ester comprises processing an alcohol production substrate to produce an alcohol composition that comprises an alcohol; providing the alcohol composition, without refining the alcohol composition, to a fatty ester production host; providing a fatty ester production substrate to the fatty ester production host; and then processing the fatty ester production substrate in the presence of the alcohol to produce a fatty ester. In some embodiments, between processing the alcohol production substrate and providing the alcohol composition to the fatty ester production host, an alcohol concentration of the alcohol composition, as measured by volume, changes by no more than about 50%, for example, about 45, 40, 35, 30, 25, 20, 15, 10, 9, 8, 7, 6, 5, 4, 3, 2, or 1%, or any amount below any of the previous values and any range defined between any of the previous two values. In some embodiments, the alcohol composition is less than about 40% by volume when provided to the fatty ester production host, for example, about 39, 38, 37, 35, 30, 25, 20, 19, 18, 17, 16, 15, 14, 13, 12, 11, 10, 9, 8, 7, 6, 5, 4, 3, 2, 1, 0.1, 0.05, 0.001%, or any amount below any of the previous values or any range defined between any of the previous two values.

In some embodiments, processing the alcohol production substrate and processing the production substrate are performed simultaneously.

In some embodiments, the alcohol comprises, consists, or consists essentially of ethanol. In some embodiments, the alcohol comprises an aliphatic group (e.g., an alkyl group) having at least 1 carbon atom. In some embodiments, the aliphatic group (e.g., an alkyl group) includes a number of carbon atoms, wherein the number of carbon atoms is selected from the group consisting of: 3, 4, 5, 6, 7, 8, 9, 10, 11, 12, 13, 14, 15, and 16. In some embodiments, the alcohol is chosen from the group consisting of ethanol, propanol, isopropanol, butanol, isobutanol, pentanol, isoamyl alcohol, isopentenol, hexanol, heptanol, octanol, nonanol, decanol, geraniol, undecanol, dodecanol, tetradecanol, pentadecanol, farnesol, and any combination thereof.

In some embodiments, the fatty ester production host is selected from the group consisting of at least one of the following: a mammalian cell, plant cell, insect cell, yeast cell, fungus cell, filamentous fungi cell, bacterial cell, a Gram-positive bacteria, a Gram-negative bacteria, genus Escherichia, genus Bacillus, genus Lactobacillus, genus Rhodococcus, genus Pseudomonas, genus Aspergillus, genus Trichoderma, genus Neurospora, genus Fusarium, genus Humicola, genus Rhizomucor, genus Kluyveromyces, genus Pichia, genus Mucor, genus Myceliophtora, genus Penicillium, genus Phanerochaete, genus Pleurotus, genus Trametes, genus Chrysosporium, genus Saccharomyces, genus Stenotrophamonas, genus Schizosaccharomyces, genus Yarrowia, genus Streptomyces, a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma-viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, a Mucor michei cell, a Streptomyces lividans cell, a Streptomyces murinus cell, an Actinomycetes cell, a CHO cell, a COS cell, a VERO cell, a BHK cell, a HeLa cell, a Cv1 cell, a MDCK cell, a 293 cell, a 3T3 cell, a PC12 cell, an E. coli cell, a strain B E. coli, a strain C E. coli, a strain K E. coli, and a strain W E. coli.

In some embodiments, the fatty ester production host comprises a recombinant cell. In some embodiments, the recombinant cell lacks a nucleic acid sequence encoding an acyl-CoA dehydrogenase enzyme (E.C. 1.3.99.3, 1.3.99.-) or wherein expression of an acyl-CoA dehydrogenase enzyme is attenuated in the recombinant cell. In some embodiments, the recombinant cell comprises a nucleic acid sequence encoding an ester synthase enzyme. In some embodiments, the recombinant cell comprises a nucleic acid sequence encoding a thioesterase enzyme. In some embodiments, the recombinant cell comprises a nucleic acid sequence encoding an acyl-CoA synthase enzyme.

In some embodiments, processing the alcohol production substrate is performed by an alcohol production host. In some embodiments, processing the alcohol production substrate is performed by a production host (e.g., an alcohol production host) selected from the group consisting of at least one of the following: Saccharomyces cerevisiae, Saccharomyces distaticus, Saccharomyces uvarum, Schizosaccharomyces pombe, Kluyveromyces marxianus, Kluyveromyces fragilis, Candida pseudotropicalis, Candida brassicae, Clostridium, Clostridium acetobutylicum, Clavispora lusitaniae, Clavispora opuntiae, Pachysolen tannophilus, Bretannomyces clausenii, Zymomonas mobilis, Zymomonas, Clostridium thermocellum, Klebsiella oxytoca, B. subtillis, yeast, Saccharomyces, Thermatoga, Bacillus, Pseudomonas, Actinomycetes, Streptomyces, Escherichia, yeast, Kluyveromyces, Candida, Clavispora, Pichia, Schizosaccharomyces, Hansenula, Pachysolen, and Bretannomyces.

In some embodiments, the specific alcohol production host can be selected to make a desired alcohol. In some embodiments, a host can be used to create iso-butanol (see, e.g., Metabolic engineering of Escherichia coli for 1-butanol production Metabolic Engineering, Available online 14 Sep. 2007 Shota Atsumi, Anthony F. Cann, Michael R. Connor, Claire R. Shen, Kevin M. Smith, Mark P. Brynildsen, Katherine J. Y. Chou, Taizo Hanai, James C. Liao). In some embodiments, Clostridia species and/or E. coli can be used to create n-butanol. In some embodiments, Clostridium, Erwinia, and/or Pseudomonas can be used to produce methanol (see, e.g., Microbial methanol formation: A major end product of pectin metabolism, Journal Current Microbiology, Issue Volume 4, Number 6/November, 1980, pages 387-389; and Microbial methanol formation: A major end product of pectin metabolism, Bernhard Schink and J. G. Zeikus.) Alcohol production hosts can also be selected for preparation of fusel alcohols and propanols. In some embodiments, the alcohol is prepared synthetically, e.g., from conversion of syn-gas (produced from, e.g., biomass, coal or oil).

In some embodiments, the alcohol production substrate comprises glucose. In some embodiments, the alcohol production substrate and the fatty ester production substrate both consist essentially of glucose. In some embodiments, the alcohol production substrate and the fatty ester production substrate are selected from the group consisting of at least one of the following: monosaccharide, glucose, fructose, mannose, galactose, oligosaccharide, fructo-oligosaccharide, galacto-oligosaccharide, polysaccharide, xylose, arabinose, disaccharide, sucrose, maltose, turanose, cellulosic material, methyl cellulose, sodium carboxymethyl cellulose, saturated or unsaturated fatty ester, succinate, lactate, acetate, starch derivatives, lignocellulosic biomass, carbon monoxide, carbon dioxide, syngas, and any combination thereof.

Fatty Esters from Impure Alcohol Compositions

While the embodiments described above focus on various impurities that result from the immediate or prior production of the alcohol itself, in some embodiments, the impurity is from a different production process that is unrelated to the synthesis of the alcohol. Thus, in some embodiments, any impure alcohol composition can be employed with the fatty ester production host(s).

In some embodiments, a production system similar to that depicted in FIG. 1 can be used to make fatty esters from impure alcohol. In some embodiments, the system is the same as depicted in FIG. 1 except that the system does not include one or more of the following aspects: alcohol production host seed vessel 128, alcohol production vessel 126, gas source 134, alcohol production subsystem 102, mechanical agitator 130, mixing valve 160, distillation process/apparatus 156, temperature sensor 154, sensor 150, production substrate storage unit 110, upstream processes 120, filtration process 144, and/or heat exchanger 140. In some embodiments, the alcohol production subsystem 102 is absent from the production system. In some embodiments, the alcohol production vessel 126 is absent.

In some embodiments, a container having an impure alcohol composition is linked to the fatty ester production vessel 164 to allow the impure alcohol composition to be delivered from the source of the impure alcohol composition to the fatty ester production vessel. In some embodiments, the impure alcohol is filtered prior to being added into the fatty ester production vessel 164. However, even when filtered, an impurity will or can remain in the impure alcohol composition prior to it being added to the fatty ester production vessel.

In some embodiments, any of the production hosts disclosed herein can be used in this process. In some embodiments, any of the production hosts described in regard to the unrefined alcohol process noted above can be used in this process as the fatty ester production host. In some embodiments, the fatty ester production host comprises a heterologous nucleic acid sequence encoding a thioesterase (e.g., EC 3.1.2.14). In some embodiments, the fatty ester production host comprises a heterologous nucleic acid sequence encoding an ester synthase (e.g., EC 2.3.1.75). In some embodiments, the fatty ester production host comprises a heterologous nucleic acid sequence encoding an acyl-CoA synthase (e.g., E.C.2.3.1.86). In some embodiments, the fatty ester production host has attenuated acyl-CoA dehydrogenase activity. In some embodiments, the fatty ester production host lacks an acyl-CoA dehydrogenase gene. In some embodiments, the fatty ester production vessel comprises a fatty ester production host comprising a heterologous nucleic acid sequence encoding an enzyme chosen from the group consisting of: thioesterase (e.g., EC 3.1.2.14), an ester synthase (e.g., EC 2.3.1.75), an alcohol acyltransferase (e.g., EC 2.3.1.84), a fatty alcohol forming acyl-CoA reductase (e.g., EC 1.1.1.*), an acyl-CoA reductase (e.g., EC 1.2.1.50), an alcohol dehydrogenase (e.g., EC 1.1.1.1), and combinations thereof.

In some embodiments, the method employed to create a fatty ester from an impure alcohol composition can be the same as described above regarding the use of an unrefined alcohol except that the steps involving the production of the alcohol need not be performed. In some embodiments, unrefined alcohol of some of the above embodiments is also an impure alcohol composition, in which case the method is the same as that outlined above. However, in other embodiments, the actual refinement and/or production of alcohol is not performed, and thus, the process can be different from other embodiments described herein.

In some embodiments, the method comprises providing an impure alcohol composition to a fatty ester production host. In some embodiments, the impure alcohol composition can be impure due to the presence of an alcohol production substrate. Thus, in some embodiments, a separate fatty ester production substrate need not be added to the fatty ester production vessel. In some embodiments, the impure alcohol is filtered and then added to the fatty ester production vessel which contains a fatty ester production substrate and a fatty ester production host. The fatty ester production host is then allowed to process the production substrate and the alcohol as noted herein. The resulting fatty esters can then be separated as described herein.

In some embodiments, the method of making a fatty ester from an impure alcohol composition comprises providing an alcohol composition to a fatty ester production host, wherein the alcohol composition contains less than about 20% alcohol by volume. One can then combine a production substrate with the fatty ester production host and allow the production host to process the production substrate in the presence of the alcohol composition to produce a fatty ester. In some embodiments, the impure alcohol composition comprises at least one alcohol selected from the group consisting of: ethanol, propanol, isopropanol, butanol, isobutanol, pentanol, isoamyl alcohol, isopentenol, hexanol, heptanol, octanol, nonanol, decanol, geraniol, undecanol, dodecanol, tetradecanol, pentadecanol, farnesol, and any combination thereof. In some embodiments, the alcohol composition comprises at least one alcohol of the formula: R₁—OH, wherein R₁ is an aliphatic group (e.g., an alkyl group) comprising a number of carbon atoms selected from the group consisting of 1, 2, 3, 4, 5, 6, 7, 8, 9, 10, 11, 12, 13, 14, and 15. In some embodiments, the production substrate is provided in the impure alcohol composition. In some embodiments, the impure alcohol composition comprises an alcohol production substrate.

In some embodiments, the fatty ester product can contain some of the original impurities from the impure alcohol composition. In some embodiments, the any one of the above methods further comprises an additional step of removing one or more impurities from a final fatty ester product. As will be appreciated by one of skill in the art, the various impurities can be removed in a variety of ways. The specific technique will depend upon the specific impurity and the properties of the specific fatty ester composition. In some embodiments, the impurity can be removed, for example, by washing, adsorption, distillation, filtration, centrifugation, settling, and/or coalescence.

In some embodiments, the impurity is removed by one of the previous steps in the production of the fatty ester. In some embodiments, the impurity is altered by the fatty ester production host or by another host in the production vessel. For example, in some embodiments, the impurity can be a sugar or some other metabolite that is altered in the fatty ester production step.

As will be appreciated by one of skill in the art, the impurity in the alcohol can be from any number of sources. In some embodiments, the impurity comprises a byproduct of the alcohol production and/or alcohol isolation. In some embodiments, the impurity is from the transportation of the alcohol composition. In some embodiments, the impurity is from the storage of the alcohol composition. In some embodiments, the impurity is from a pre-fatty ester processing step.

In some embodiments, the impurity is present due to the alcohol production, but is not present in production processes that employ a fatty ester production host.

While there are a number of impurities that can be present in the alcohol or the final fatty ester product, in some embodiments, one or more of the following impurities is present in a detectable amount: sugar, mannitol, cellulose, hemicelluloses, starch, soluble polysaccharides, dextran, phytoglycogen, potassium, sodium, calcium, magnesium, chlorides, bicarbonate, sulfate, phosphate, iron, aluminum, silica, ammonium, nitrate, ketones, polyols, dihydroxyacetone, furfural, hydroxymethylfurfural, Amadori products, Heyns products, pyrrole derivatives, pyridine derivatives, imidazole derivatives, pyrazine derivatives, heterocylcic caramel products, alicyclic caramel products, H-bonded caramel products, phenolic based colors, cis-aconitic acid, trans-aconitic acid, tartaric acid, citric acid, fumaric acid, malic acid, succinic acid, shikimic acid, 2,4-dihydroxybutyric acid, methylglyceric acid, saccharinic acids, palmitic acid, oleic acid, linoleic acid, linolenic acid, acetic acid, lactic acid, formic acid, glyceric acid, oxalic acid, glycolic acid, aromatic acids, ferulic acid, p-hydroxybenzoic acid, vanillic acid, caffeic acid, p-coumaric acid, 3,4-dihydroxybenzoic acid, 2,3-dihydroxybenzoic acid, phenolics, lignin, chlorogenic acid, neutral phenolics, glycosidic flavinoids, luteolins, 6-methoxyluteolin, apigenins, tricins, fats, phosphatides, chlorophyll A, chlorophyll B, carotene, xanthophyll, anthocyanins, phosphatidylethanolamine, lecithin, vitamins, thiamine, riboflavin, pyridoxine (B6 group), niacin, calcium pantothenate, biotin, folic acid, betaine, amides, acetamide, lactamide, N-sugar color, pyrollidone carboxylic acid (PCA), allantoin, allantoic acid, aspartic acid, asparagine, asparagine, glutamic acid, glutamine, glutamine, α-alanine, valine, γ-aminobutyric acid, threonine, isoleucine, glycine, leucine, lysine, serine, arginine, phenylalanine, tyrosine, histidine, hydroxyproline, proline, methionine, tryptophan, uridine, adenine, pesticides, herbicides, aldrin, dieldrin, chlordane, trehalose, acetaldehyde, acetals, 3-methyl-1-butanol, 2-methyl-1-propanol, 2-propanol, 1-propanol, 1-butanol, 2-methylbutanol, sulfite waste liquor, fusel alcohols, n-pentanol, n-hexanol, n-heptanol, higher straight-chain aldehydes, pentanal, hexanal, heptanal, octanal, aromatic alcohols, phenol derivatives, silica, maillard, caramel color, organic acids, aromatic acids, polypeptides, nucleic acids, fructose, iso-maltose, sorbitol, erythritol, glycerol, lactobacillus, and combinations thereof.

Impurities from industrial ethanol from quick cane juice fermentation, include, for example, mannitol, polysaccharides, cations (Ca⁺² Mg⁺² Na⁺ K⁺, iron, aluminum), sulfate, phosphate, chloride, silica, maillard and caramel color, organic acids (C4-C6), fatty acids, organics acids (C2-C3), aromatic acids, phenolics/lignins, other fatty species, vitamins, polypeptides, amino acids, and nucleic acids (as well as combinations thereof).

Impurities can be found in crude ethanol due to alcohol fermentation or industrial ethanol fermentation. Impurities related to raw sugar, processing of the raw sugar, or to the yeast fermentation can be present. Impurities due to sugar processing include glucose, fructose, and iso-maltose.

Impurities can include those from yeast reactions and byproducts of yeast reactions. Such impurities can include, for example, acetaldehyde, acetic acid, and trehalose (e.g., 200 to 400 ppm of trehalose and 50 to 200 ppm isomaltose). Impurities can include polyols such as sorbitol, erythritol, and mannitol (e.g., concentrations of 5 to 50 ppm). Impurities can include glycerol. Glycerol can be present at levels of 10 g/L or more. Impurities can also include monoglycerides (e.g., at up to 10% by volume of the concentration of the ethanol-fatty esters). Other impurities include organic acids, Lactobacillus, and other contaminants present during the ethanol fermentation, lactic acid and succinic acid, and acetic acid.

Additional fusels can be found for example at Appl. Environ. Microbiol. doi:10.1128/AEM.02625-07 “The Ehrlich pathway for fusel alcohol production: a century of research on yeast metabolism” by Lucie A. Hazelwood, Jean-Marc Daran, Antonius J. A. van Maris, Jack T. Pronk, and J. Richard Dickinson, the entirety of which is incorporated herein by reference.

In some embodiments, the impurity is a molecule that, while not present in a standard fatty ester production process, is present in an alcohol or ethanol production process, and, thus, can be present in the fatty ester production process disclosed herein.

In some embodiments, the liquid comprising the alcohol (i.e., the alcohol composition) that is added to the fatty ester production vessel is less than 100% alcohol. The amount of the impurity will be less than 100% by volume of the alcohol composition. For example, in some embodiments, the impurity is about 99, 95, 90, 80, 70, 60, 50, 40, 30, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, or 0.1 percent of the alcohol composition.

In some embodiments, an impurity is at least 0.1% of the volume of the impure alcohol composition, for example, about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 30, 40, 50, 60, 70, 80, 90, 95, 98, or 99 percent of the volume of the alcohol composition, including any amount above or below the previous numbers or any amount defined between any two of the previous numbers.

In some embodiments, an impurity is at least 0.1% of the volume of the fatty ester product that is created when the fatty ester produced by the production host mixes with the solution containing the production host. In some embodiments, the impurity in the fatty ester product is at least, for example, about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 30, 40, 50, 60, 70, 80, 90, 95, 98, or 99 percent of the volume in the fatty ester production vessel, including any amount above or below the previous numbers or any amount defined between any two of the previous numbers.

In some embodiments, more than one type of impurity is present in any of the above amounts. In some embodiments, at least about 2, 3, 4, 5, 6, 7, 8, 9, 10, 11, 12, 13, 14, 15, 16, 17, 18, 19, 20, 25, 30, 35, 40, 50, 60, 70, 80, 90, or 100 different impurities in detectable amounts are present in a single impure alcohol composition.

In some embodiments, even in the presence of one or more of the above impurities at one of the above impurity concentrations, the presently disclosed process using the production hosts described herein can result in the significant production of fatty esters, for example, the fatty ester production process can produce at least about 0.0001, 0.001, 0.01, 0.1, 1, 5, 10, 20, 50, 70, 80, 90, 95, 98, 99, 100% as much fatty ester as the same production process (using the same production hosts), but without the one or more impurities at the relevant concentration. In some embodiments, the production hosts can be as efficient, even in the presence of the impurity. Thus, in some embodiments, the production host can produce any of the above amounts of the fatty ester under the same conditions and in the same time. In some embodiments, the time it takes to produce the same amount of fatty ester, while longer than in the absence of an impurity, is not prohibitively longer. Thus, in some embodiments, the amount of the fatty ester is produced in at least the same amount of time, including, for example, about 100, 120, 150, 200, 300, 400, 500, 800, or 1000 percent of the amount of time it takes to make the fatty esters without the impurity present.

In some embodiments, the impurity is acceptable for use in various applications, including, for example, for use in various biofuels, such as biodiesels. Thus, in some embodiments, the impurity can be present in a useable biofuel (e.g., biodiesel). In some embodiments, the retained impurity (an impurity that is in both the initial alcohol composition and a final fatty ester product) is one that is not detrimental to the device or use for which the biofuel (e.g., biodiesel) is to be used. In some embodiments, the impurity is one that will not harm a biofuel (e.g., biodiesel) engine any more than a pure fatty ester composition. In some embodiments, the impurity is as volatile as the fatty ester and acts as a source of energy in the biofuel (e.g., biodiesel), similar to the fatty ester. In some embodiments, the amount of the impurity is substantially low so as to allow the device using the fatty ester composition to still operate.

In some embodiments, an impurity is at least about 0.1% of the volume of the refined fatty ester product (including, for example, a biofuel (e.g., biodiesel)). In some embodiments, the impurity in the refined fatty ester product is at least, for example, about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 30, 40, 50, 60, 70, 80, 90, 95, 98, or 99 percent of the volume of the refined fatty ester product.

In some embodiments, the alcohol is synthetic and can be produced from gas (e.g., from biomass, coal or oil). Thus, in some embodiments, the impurities in the alcohol are produced from such a process. Such processes are disclosed in U.S. Pat. Nos. 7,288,689, and 5,856,592, the entireties of which are incorporated by reference.

In some embodiments, the impure alcohol composition source is a crude methanol produced from syn-gas. For example a syn-gas can be compressed and then fed into a converter column containing a catalyst, producing methanol with several impurities. Traditionally, in a methanol production process, such crude methanol is purified by distillation to remove these trace ethers and hydrocarbon impurities, as well as water. In some of the present embodiments, the crude methanol can be fed undistilled or partially distilled to the fatty ester production hosts.

In some embodiments, the syngas can be derived from various biomass gasification techniques. In some embodiments, the method or device to produce the fatty ester employs an impure or only partially purified syngas.

### Impure Fatty Ester Products

In some embodiments, the fatty ester composition produced from an impure alcohol or via one of the herein disclosed methods comprises an alcohol production host and a fatty ester. The fatty ester can be a significant amount of the composition. For example, the fatty ester can be at least about 10, 20, 30, 40, 50, 60, 70, 80, 90, 85% or more of the impure fatty ester composition. In some embodiments, the alcohol production host is a yeast. In some embodiments, the yeast is Saccharomyces cerevisiae. In some embodiments, the alcohol production host is selected from the group of Saccharomyces cerevisiae, Saccharomyces distaticus, Saccharomyces uvarum, Schizosaccharomyces pombe, Kluyveromyces marxianus, Kluyveromyces fragilis, Candida pseudotropicalis, Candida brassicae, the genus Clostridium, Clostridium acetobutylicum, Clavispora lusitaniae, Clavispora opuntiae, Pachysolen tannophilus, Bretannomyces clausenii, Zymomonas mobilis, the genus Zymomonas, Clostridium thermocellum, Klebsiella oxytoca, Bacillus subtillis, yeast, the genus Saccharomyces, the genus Thermatoga, the genus Bacillus, the genus Pseudomonas, the genus Actinomycetes, the genus Streptomyces, the genus Escherichia the genus Kluyveromyces, the genus Candida, the genus Clavispora, the genus Pichia, the genus Schizosaccharomyces, the genus Hansenula, the genus Pachysolen, and the genus Bretannomyces, and any combination thereof. In some embodiments, the alcohol production host is a bacterium. In some embodiments, the bacterium is from a genus chosen from the group of Zymomonas, Clostridium, Escherichia, and any combination thereof. In some embodiments, the bacterium is Escherichia coli. In some embodiments, the fatty ester is or comprises an ethyl ester.

In some embodiments, the fatty ester is part of a fatty ester composition of a biofuel, such as a biodiesel. In some embodiments, the fatty ester composition comprises a fatty ester and a detectable amount of at least one impurity selected from the group consisting of: mannitol, cellulose, hemicelluloses, starch, soluble polysaccharides, dextran, phytoglycogen, potassium, sodium, calcium, magnesium, chlorides, bicarbonate, sulfate, phosphate, iron, aluminum, silica, ammonium, nitrate, ketones, polyols, dihydroxyacetone, furfural, hydroxymethylfurfural, Amadori products, Heyns products, pyrrole derivatives, pyridine derivatives, imidazole derivatives, pyrazine derivatives, heterocylcic caramel products, alicyclic caramel products, H-bonded caramel products, phenolic based colors, cis-aconitic acid, trans-aconitic acid, tartaric acid, citric acid, fumaric acid, malic acid, succinic acid, shikimic acid, 2,4-dihydroxybutyric acid, methylglyceric acid, saccharinic acids, palmitic acid, oleic acid, linoleic acid, linolenic acid, acetic acid, lactic acid, formic acid, glyceric acid, oxalic acid, glycolic acid, aromatic acids, ferulic acid, p-hydroxybenzoic acid, vanillic acid, caffeic acid, p-coumaric acid, 3,4-dihydroxybenzoic acid, 2,3-dihydroxybenzoic acid, phenolics, lignin, chlorogenic acid, neutral phenolics, glycosidic flavinoids, luteolins, 6-methoxyluteolin, apigenins, tricins, fats, phosphatides, chlorophyll A, chlorophyll B, carotene, xanthophyll, anthocyanins, phosphatidylethanolamine, lecithin, vitamins, thiamine, riboflavin, pyridoxine (B6 group), niacin, calcium pantothenate, biotin, folic acid, betaine, amides, acetamide, lactamide, N-sugar color, pyrollidone carboxylic acid (PCA), allantoin, allantoic acid, aspartic acid, asparagine, asparagine, glutamic acid, glutamine, glutamine, α-alanine, valine, γ-aminobutyric acid, threonine, isoleucine, glycine, leucine, lysine, serine, arginine, phenylalanine, tyrosine, histidine, hydroxyproline, proline, methionine, tryptophan, uridine, adenine, pesticides, herbicides, aldrin, dieldrin, chlordane, trehalose, acetaldehyde, acetals, 3-methyl-1-butanol, 2-methyl-1-propanol, 2-propanol, 1-propanol, 1-butanol, 2-methylbutanol, sulfite waste liquor, fusel alcohols, n-pentanol, n-hexanol, n-heptanol, higher straight-chain aldehydes, pentanal, hexanal, heptanal, octanal, aromatic alcohols, phenol derivatives, silica, maillard, caramel color, organic acids, aromatic acids, polypeptides, nucleic acids, fructose, iso-maltose, sorbitol, erythritol, glycerol, lactobacillus, and combinations thereof. In some embodiments, the fatty ester composition comprises one or more of 3-methyl-1-butanol, 2-methyl-1-propanol (isobutanol), 2-propanol, 1-propanol, 1-butanol, 2-methylbutanol, fusel alcohols, n-pentanol, n-hexanol, n-heptanol, and combinations thereof.

In some embodiments, the fatty ester composition further comprises an additional impurity comprising a starch. In some embodiments, the starch is derived from a plant selected from the group consisting of barley, corn, wheat, potato, and rice. In some embodiments, the fatty ester has a fraction of modern carbon of about 1.003 to about 1.5. In some embodiments, the fatty ester composition comprises a mixture of fatty esters selected from the group consisting of: C12:0, C12:1, C14:0, C14:1, C16:0, C16:1, C18:0, and C18:1. In an alternate embodiment, at least 60% by volume of the fatty esters are C16, C18, or some combination thereof.

In some embodiments, the fatty ester has the following formula:

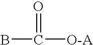

The B side of the fatty ester can be a carbon chain comprising at least 6 carbons and the A side can be an aliphatic group (e.g., an alkyl group) that comprises at least one carbon. In some embodiments, the B side of the fatty ester is a polyunsaturated carbon chain. In some embodiments, the B side of the fatty ester is a monounsaturated carbon chain. In some embodiments, the aliphatic group (e.g., an alkyl group) of the A side of the fatty ester has a number of carbon atoms selected from the group consisting of: 1, 2, 3, 4, 5, 6, 7, 8, 9, 10, 11, 12, 13, 14, 15, 16, and 17 carbon atoms. In some embodiments, the B side of the fatty ester comprises a carbon chain having a number of carbon atoms selected from the group consisting of: 5, 6, 7, 8, 9, 10, 11, 12, 13, 14, 15, 16, 17, 18, 19, 20, 21, 22, 23, 24, 25, 26, 27, 28, 29, and 30 carbon atoms. In some embodiments, the number of carbon atoms comprising the fatty ester is selected from the group consisting of 16, 17, and 18 carbon atoms. In some embodiments, the fatty ester has a δ¹³ of from about −10.9 to about −15.4. In some embodiments, the fatty ester has a fraction of modern carbon of about 1.003 to about 1.5. In some embodiments, the fatty ester has a δ¹³ of about −28 or greater, for example, a δ¹³ of about −18 or greater, a δ¹³ of about −27 to about −24, or a δ¹³ of about −16 to about −10. In some embodiments, the fatty ester has a f_{M} ¹⁴C of at least about 1, for example, a f_{M} ¹⁴C of at least about 1.01, a f_{M} ¹⁴C of about 1 to about 1.5, a f_{M} ¹⁴C of about 1.04 to about 1.18, or a f_{M} ¹⁴C of about 1.111 to about 1.124.

### Production Hosts for the Production of Fatty Acid Derivatives and Fatty Esters

As noted above, production hosts are cells that can be used to convert a production substrate into a product, such as a fatty ester. Examples of production hosts include plant, animal, human, bacteria, yeast, and/or filamentous fungi cells.

In some embodiments, the production hosts comprise heterologous nucleic acid sequences or lack native nucleic acid sequences. In some embodiments, the production host comprises a heterologous nucleic acid sequence encoding a thioesterase (e.g., EC 3.1.2.14). In some embodiments, the production host comprises a heterologous nucleic acid sequence encoding an ester synthase (e.g., EC 2.3.1.75). In some embodiments, the production host comprises a heterologous nucleic acid sequence encoding an acyl-CoA synthase (e.g., E.C.2.3.1.86). In some embodiments, the production host lacks a nucleic acid sequence encoding an acyl-CoA dehydrogenase enzyme. In some embodiments, the production host expresses an attenuated level of an acyl-CoA dehydrogenase enzyme. In some embodiments, any combination of the above is present in a host.

In some embodiments, the production host comprises a heterologous nucleic acid sequence encoding an alcohol acetyltransferase (e.g., EC 2.3.1.84). In some embodiments, the production host comprises a heterologous nucleic acid sequence encoding a fatty alcohol forming acyl-CoA reductase (e.g., EC 1.1.1.*) (wherein “*” denotes that any option applies at this position). In some embodiments, the production host comprises a heterologous nucleic acid sequence encoding an acyl-CoA reductase (e.g., EC 1.2.1.50). In some embodiments, any combination of the above is present in a host.

In some embodiments, fatty alcohols having defined carbon chain lengths can be produced by expressing particular exogenous nucleic acid sequences encoding thioesterases (e.g., EC 3.1.2.14) and combinations of acyl-CoA reductases (e.g., EC 1.2.1.50), alcohol dehydrogenases (e.g., EC 1.1.1.1) and fatty alcohol forming acyl-CoA reductases (e.g., EC 1.1.1*). Other enzymes that can be also modulated to increase the production of fatty alcohols include enzymes involved in fatty acid synthesis (e.g., EC 2.3.1.85) and acyl-CoA synthase (e.g., EC 2.3.1.86).

In some embodiments, fatty esters having defined carbon chain lengths can be produced by exogenously expressing particular thioesterases (e.g., EC 3.1.2.14), combinations of acyl-CoA reductase (1.2.1.50), alcohol dehydrogenases (EC 1.1.1.1) and fatty alcohol forming acyl-CoA reductase (e.g., EC 1.1.1*), as well as, acetyl transferase (e.g., EC 2.3.1.84). Other enzymes that can be modulated to increase the production of fatty esters include enzymes involved in fatty acid synthesis (e.g., EC 2.3.1.85) and acyl-CoA synthase (e.g., EC 2.3.1.86).

In some embodiments, fatty esters can be produced by adding exogenous alcohols to the medium.

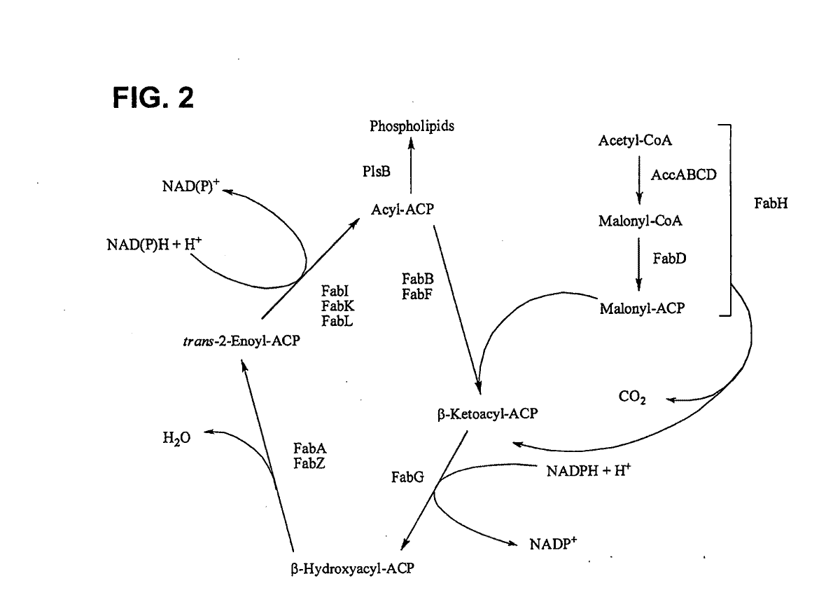

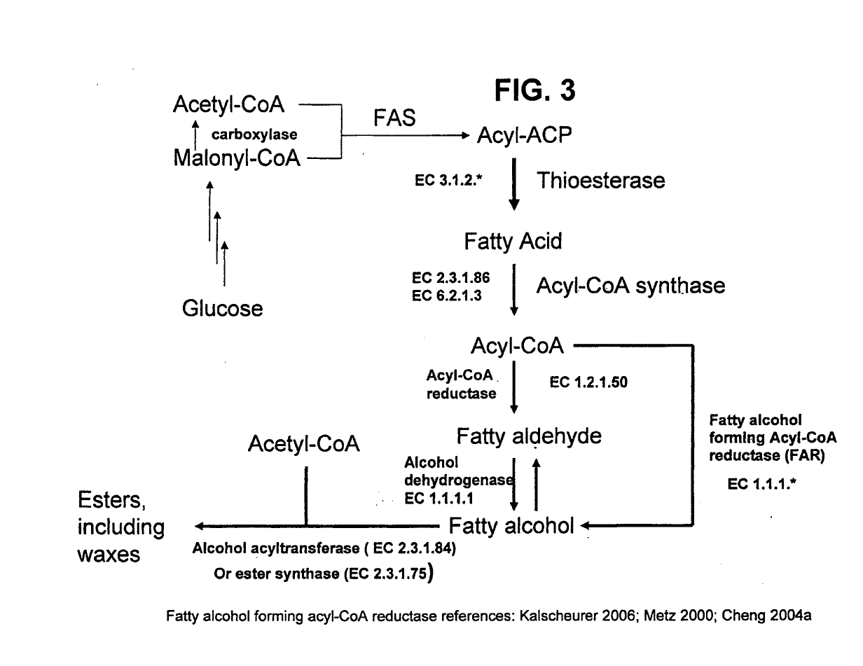

In some embodiments, the host organism that heterologous DNA sequences are transformed into can be a modified host organism, such as an organism that has been modified to increase the production of acyl-ACP or acyl-CoA, reduce the catabolism of fatty acid derivatives and intermediates, or to reduce feedback inhibition at specific points in the biosynthetic pathway. In addition to modifying the genes described herein, additional cellular resources can be diverted to over produce fatty acids. For example, the lactate, succinate and/or acetate pathways can be attenuated or acetyl-CoA carboxylase (ACC) can be over expressed. The modifications to the production host described herein can be through genomic alterations, extrachromosomal expression systems, or combinations thereof. An overview of one such pathway is provided in FIGS. 2 and 3.

Acetyl-CoA-Malonyl-CoA to Acyl-ACP

Fatty acid synthase (FAS) is a group of enzymes that catalyze the initiation and elongation of acyl chains. The acyl carrier protein (ACP) along with the enzymes in the FAS pathway control the length, degree of saturation, and branching of the fatty acids produced. Enzymes that can be included in FAS include AccABCD, FabD, FabH, FabG, FabA, FabZ, FabI, FabK, FabL, FabM, FabB, and FabF. Depending upon the desired product one or more of these genes can be attenuated or over-expressed.

In some embodiments, the fatty acid biosynthetic pathway in the production host uses the precursors acetyl-CoA and malonyl-CoA (FIG. 3). E. coli or other host organisms engineered to overproduce these components can serve as the starting point for subsequent genetic engineering steps to provide the specific output product (such as, fatty esters, hydrocarbons, fatty alcohols). Several different modifications can be made, either in combination or individually, to the host strain to obtain increased acetyl-CoA/malonyl-CoA/fatty acid and fatty acid derivative production. For example, to increase acetyl-CoA production, a plasmid with pdh, panK, aceEF (encoding the E1p dehydrogenase component and the E2p dihydrolipoamide acyltransferase component of the pyruvate and 2-oxoglutarate dehydrogenase complexes), fabH/fabD/fabG/acpP/fabF, and in some examples additional DNA encoding fatty acyl-CoA reductases and aldehyde decarbonylases, all under the control of a constitutive, or otherwise controllable promoter, can be constructed. Exemplary Genbank accession numbers for these genes are: pdh (BAB34380, AAC73227, AAC73226), panK (also known as conA, AAC76952), aceEF (AAC73227, AAC73226), fabH (AAC74175), fabD (AAC74176), fabG (AAC74177), acpP (AAC74178), fabF (AAC74179).

Additionally, fadE, gpsA, ldhA, pflb, adhE, pta, poxB, ackA, and/or ackB can be knocked-out or their expression levels can be reduced in the engineered microorganism. This can be accomplished by transformation with conditionally replicative or non-replicative plasmids containing null or deletion mutations of the corresponding genes or by substituting promoter or enhancer sequences. Exemplary Genbank accession numbers for these genes are; fadE (AAC73325), gspA (AAC76632), IdhA (AAC74462), pflb (AAC73989), adhE (AAC74323), pta (AAC75357), poxB (AAC73958), ackA (AAC75356), and ackB (BAB81430).

The resulting engineered microorganisms can be grown in a desired environment, for example, one with limited glycerol (e.g., less than 1% w/v in the culture medium). By doing this, these microorganisms will have increased acetyl-CoA production levels. Malonyl-CoA overproduction can be affected by engineering the microorganism, as described above, with DNA encoding accABCD (acetyl-CoA carboxylase, accession number AAC73296, EC 6.4.1.2). Fatty acid overproduction can be achieved by further including DNA encoding lipase (for example, Accessions numbers CAA89087, CAA98876).

In some examples, acetyl-CoA carboxylase (acc) is over-expressed to increase the intracellular concentration thereof by at least 2-fold, such as at least 5-fold, or at least 10-fold relative to native expression levels.

In addition, the plsB (for example, Accession number AAC77011) D311E mutation can be used to remove limitations on the pool of acyl-CoA.

In addition, over-expression of a sfa gene (suppressor of FabA Accession number AAN79592) can be included in the production host to increase production of monounsaturated fatty acids (see, e.g., Rock et al., J. Bacteriology 178:5382-5387, 1996).

Acyl-ACP to Fatty Acid

To engineer a production host for the production of a homogeneous population of fatty acid derivatives, one or more endogenous genes can be attenuated or functionally deleted. In addition, one or more thioesterases can be expressed. For example, C10 fatty acid derivatives can be produced by attenuating thioesterase C18 (for example, accession numbers AAC73596 and P0ADA1), which uses C18:1-ACP and expressing thioesterase C10 (for example, accession number Q39513), which uses C10-ACP. This results in a relatively homogeneous population of fatty acid derivatives that have a carbon chain length of 10. In another example, C14 fatty acid derivatives can be produced by attenuating endogenous thioesterases that produce non-C14 fatty acids and expressing the thioesterase accession number Q39473 (which uses C14-ACP). In yet another example, C12 fatty acid derivatives can be produced by expressing thioesterases that use C12-ACP (for example, accession number Q41635) and attenuating thioesterases that produce non-C12 fatty acids. acetyl-CoA, malonyl-CoA, and fatty acid overproduction can be verified using methods known in the art, for example, by using radioactive precursors, HPLC, and GC-MS subsequent to cell lysis.

TABLE 1
Thioesterases

| | | | Preferential |
|---|---|---|---|
| Accession | | | product |
| Number | Source Organism | Gene | produced |
| AAC73596 | E. coli | tesA without leader | C18:1 |
| | | sequence | |
| Q41635 | Umbellularia | fatB | C12:0 |
| | california | | |
| Q39513; | Cuphea hookeriana | fatB2 | C8:0-C10:0 |
| AAC49269 | Cuphea hookeriana | fatB3 | C14:0-C16:0 |
| Q39473 | Cinnamonum | fatB | C14:0 |
| | camphorum | | |
| CAA85388 | Arabidopsis thaliana | fatB[M141T]* | C16:1 |
| NP 189147; | Arabidopsis thaliana | fatA | C18:1 |
| NP 193041 | | | |
| CAC39106 | Bradyrhiizobium | fatA | C18:1 |
| | japonicum | | |
| AAC72883 | Cuphea hookeriana | fatA | C18:1 |

*Mayer et al., BMC Plant Biology 7: 1-11, 2007.

Fatty Acid to Acyl-CoA

Production hosts can be engineered using known peptides to produce fatty acids of various lengths. One method of making fatty acids involves increasing the expression of, or expressing more active forms of, one or more acyl-CoA synthases (e.g., EC 2.3.1.86).

As used herein, acyl-CoA synthase includes enzymes in enzyme classification number EC 2.3.1.86, as well as any other enzymes capable of catalyzing the conversion of a fatty acid to an acyl-CoA. Additionally, one of ordinary skill in the art will appreciate that some acyl-CoA synthases will catalyze other reactions as well. For example some acyl-CoA synthases will accept other substrates in addition to fatty acids. Such non-specific acyl-CoA synthase peptides are, therefore, also included. Acyl-CoA synthase sequences are publicly available. Exemplary GenBank Accession Numbers are provided in FIG. 9.

Acyl-CoA to Fatty Alcohol

Production hosts can be engineered using known polypeptides to produce fatty alcohols from acyl-CoA. One method of making fatty alcohols involves increasing the expression of, or expressing more active forms of, fatty alcohol forming acyl-CoA reductases (e.g., EC 1.1.1.*) acyl-CoA reductases (e.g., EC 1.2.1.50), or alcohol dehydrogenases (e.g., EC 1.1.1.1). Hereinafter, fatty alcohol forming acyl-CoA reductases (e.g., EC 1.1.1.*), acyl-CoA reductases (e.g., EC 1.2.1.50), and alcohol dehydrogenases (e.g., EC 1.1.1.1) are collectively referred to as fatty alcohol forming enzymes. In some examples, all three of the fatty alcohol forming genes can be over expressed in a production host. In yet other examples, one or more of the fatty alcohol forming genes can be over-expressed.

As used herein, fatty alcohol forming peptides include peptides in enzyme classification numbers EC 1.1.1.*, 1.2.1.50, and 1.1.1.1, as well as any other peptide capable of catalyzing the conversion of acyl-CoA to fatty alcohol. Additionally, one of ordinary skill in the art will appreciate that some fatty alcohol forming peptides will catalyze other reactions as well. For example, some acyl-CoA reductases will accept other substrates in addition to fatty acids. Such non-specific peptides are, therefore, also included. Fatty alcohol forming peptide sequences are publicly available. Exemplary GenBank Accession Numbers are provided in FIG. 9.

In some embodiments, a microorganism can be engineered to produce fatty alcohols by including a first exogenous DNA sequence encoding a protein capable of converting a fatty acid to a fatty aldehyde and a second exogenous DNA sequence encoding a protein capable of converting a fatty aldehyde to an alcohol. In some examples, the first exogenous DNA sequence encodes a fatty acid reductase. In one embodiment, the second exogenous DNA sequence encodes a mammalian microsomal aldehyde reductase or a long-chain aldehyde dehydrogenase. In a further example, the first and second exogenous DNA sequences are from a multienzyme complex from Arthrobacter AK 19, Rhodotorula glutinins, Acinobacter sp strain M-1, or Candida lipolytica. In one embodiment, the first and second heterologous DNA sequences are from a multienzyme complex from Acinobacter sp strain M-1 or Candida lipolytica.

Additional sources of heterologous DNA sequences encoding enzymes which convert a fatty acid to a long chain alcohol include, but are not limited to, Mortierella alpina (ATCC 32222), Crytococcus curvatus, (also referred to as Apiotricum curvatum), Alcanivorax jadensis (T9T=DSM 12718=ATCC 700854), Acinetobacter sp. HOl-N, (ATCC 14987) and Rhodococcus opacus (PD630 DSMZ 44193).

In one example, the fatty acid derivative is a saturated or unsaturated fatty alcohol having a carbon atom content limited to between 6 and 36 carbon atoms. In another example, the fatty alcohol has a carbon atom content limited to between 24 and 32 carbon atoms.

Appropriate hosts for producing s fatty alcohols can be either eukaryotic or prokaryotic microorganisms. Exemplary hosts include Arthrobacter AK 19, Rhodotorula glutinins, Acinobacter sp strain M-1, Arabidopsis thalania, or Candida lipolytica, Saccharomyces cerevisiae, and E. coli engineered to express acetyl-CoA carboxylase. Hosts which demonstrate an innate ability to synthesize high levels of fatty alcohol precursors in the form of lipids and oils, such as Rhodococcus opacus, Arthrobacter AK 19, Rhodotorula glutinins, E. coli engineered to express acetyl-CoA carboxylase, or other oleaginous bacteria, yeast, and fungi can also be used.

Fatty Alcohols to Fatty Esters

Production hosts can be engineered to produce fatty esters of various lengths. One method of making fatty esters includes increasing the expression of, or expressing more active forms of, one or more alcohol O-acetyltransferase peptides (e.g., EC 2.3.1.84). These peptides catalyze the reaction of acetyl-CoA and an alcohol to form CoA and a fatty ester. In some examples, the alcohol O-acetyltransferase peptides can be expressed in conjunction with selected thioesterase peptides, FAS peptides, and fatty alcohol forming peptides. This allows the carbon chain length, saturation, and degree of branching to be controlled. In some cases, the bkd operon can be coexpressed to enable branched fatty acid precursors to be produced.

As used herein, alcohol O-acetyltransferase peptides include peptides in enzyme classification number EC 2.3.1.84, as well as any other peptide capable of catalyzing the conversion of acetyl-CoA and an alcohol to form CoA and acetic fatty ester. Additionally, one of ordinary skill in the art will appreciate that alcohol O-acetyltransferase peptides will catalyze other reactions as well. For example, some alcohol O-acetyltransferase peptides will accept other substrates in addition to fatty alcohols or acetyl-CoA thioester (i.e., other alcohols and other acyl-CoA thioesters). Such non-specific or divergent specific alcohol O-acetyltransferase peptides are, therefore, also included. Alcohol O-acetyltransferase peptide sequences are publicly available. Exemplary GenBank Accession Numbers are provided in FIG. 9. Assays for characterizing the activity of a particular alcohol O-acetyltransferase peptides are well known in the art. Engineered O-acetyltransferases and O-acyltransferases can be also created that have new activities and specificities for the donor acyl group or acceptor alcohol moiety. Engineered enzymes could be generated through rational and evolutionary approaches well documented in the art.

In some embodiments, the expression of exogenous FAS genes originating from different species or engineered variants can be introduced into the host cell to result in the biosynthesis of fatty acid metabolites structurally different (e.g., in length, branching, degree of unsaturation, etc.) than that of the native host. These heterologous gene products can be also chosen or engineered so that they are unaffected by the natural regulatory mechanisms in the host cell and, therefore, function in a manner that is more controllable for the production of the desired commercial product. For example, the FAS enzymes from Bacillus subtilis, Saccharomyces cerevisiae, Streptomyces spp, Ralstonia, Rhodococcus, Corynebacteria, Brevibacteria, Mycobacteria, oleaginous yeast, and the like can be expressed in the production host.

One of ordinary skill in the art will appreciate that when a production host is engineered to produce a fatty acid from the fatty acid biosynthetic pathway that contains a specific level of unsaturation, branching, or carbon chain length, the resulting engineered fatty acid can be used in the production of the fatty acid derivatives. Hence, fatty acid derivatives generated from the production host can display the characteristics of the engineered fatty acid. For example, a production host can be engineered to make branched, short chain fatty acids, and then using the teachings provided herein relating to fatty alcohol production (e.g., including alcohol forming enzymes, such as FAR) the production host produces branched, short chain fatty alcohols. Similarly, a hydrocarbon can be produced by engineering a production host to produce a fatty ester having a defined level of branching, unsaturation, and/or carbon chain length, thus, producing a homogenous hydrocarbon population. Moreover, when an unsaturated alcohol, fatty ester, or hydrocarbon is desired the fatty acid biosynthetic pathway can be engineered to produce low levels of saturated fatty acids and an additional desaturase can be expressed to lessen the saturated product production.

In some embodiments, the fatty ester production host will include an ester synthase. As used herein, ester synthases includes enzymes in enzyme classification number EC 2.3.1.75, as well as any other peptide capable of catalyzing the conversion of an acyl-thioester to fatty esters. Additionally, one of ordinary skill in the art will appreciate that some ester synthases will catalyze other reactions as well. For example, some ester synthases will accept short chain acyl-CoAs and short chain alcohols and produce fatty esters. Such non-specific ester synthases are, therefore, also included. Ester synthase sequences are publicly available. Exemplary GenBank Accession Numbers are provided in FIG. 9. Methods to identify ester synthase activity are provided in U.S. Pat. No. 7,118,896, which is herein incorporated by reference.

In some embodiments, if the desired product is a fatty ester based biofuel, the microorganism is modified so that it produces a fatty ester generated from a renewable energy source. Such a microorganism includes a heterologous DNA sequence encoding an ester synthase that is expressed so as to confer upon said microorganism the ability to synthesize a saturated, unsaturated, or branched fatty ester from a renewable energy source. In some embodiments, the ester synthases include, but are not limited to: fatty acid elongases, acyl-CoA reductases, acyltransferases, ester synthases, fatty acyl transferases, diacylglycerol acyltransferases, acyl-coA wax alcohol acyltransferases, or bifunctional ester synthase/acyl-CoA:diacylglycerol acyltransferases. Bifunctional ester synthase/acyl-CoA:diacylglycerol acyltransferases can be selected from a multienzyme complex from Simmondsia chinensis, Acinetobacter sp. strain ADP1 (formerly Acinetobacter calcoaceticus ADP1), Pseudomonas aeruginosa, Fundibacter jadensis, Arabidopsis thaliana, or Alkaligenes eutrophus. In one embodiment, the fatty acid elongases, acyl-CoA reductases, or ester synthases are from a multienzyme complex from Alkaligenes eutrophus and other organisms known in the literature to produce fatty esters. Additional sources of heterologous DNA encoding ester synthases useful in fatty ester production include, but are not limited to, Mortierella alpina (for example ATCC 32222), Crytococcus curvatus, (also referred to as Apiotricum curvatum), Alcanivorax jadensis (for example T9T=DSM 12718=ATCC 700854), Acinetobacter sp. HOl-N, (for example ATCC 14987), and Rhodococcus opacus (for example PD630, DSMZ 44193).

In some embodiments, useful hosts for producing fatty esters can be either eukaryotic or prokaryotic microorganisms. In some preferred embodiments such hosts include, but are not limited to, Saccharomyces cerevisiae, Candida lipolytica, E. coli Arthrobacter AK 19, Rhodotorula glutinins, Acinobacter sp strain M-1, Candida lipolytica, and other oleaginous microorganisms. Given their high lipid content, fatty acid content, and precursors which can be converted to fatty esters, the preferred hosts are E. coli and Candida lipolytica.

In some embodiments, the ester synthase from Acinetobacter sp. ADP1 (e.g., at locus AAO17391 (described in Kalscheuer and Steinbuchel, J. Biol. Chem. 278:8075-8082; (2003, herein incorporated by reference)) is used. In some embodiments, the ester synthase from Simmondsia chinensis (e,g., at locus AAD38041) is used.

In some embodiments, an ester exporter, such as a member of the FATP family, is used to facilitate the release of fatty esters into the extracellular environment. One example of an ester exporter that can be used is fatty acid (long chain) transport protein CG7400-PA, isoform A from Drosophila melanogaster (e.g., at locus NP_{—}524723).

Genetic Engineering to Increase Fatty Acid Derivative Production

In some embodiments, heterologous DNA sequences involved in biosynthetic pathways for the production of fatty acid derivatives or fatty esters can be introduced stably or transiently into a production host cell using established techniques well known in the art including, for example, electroporation, calcium phosphate precipitation, DEAE-dextran mediated transfection, liposome-mediated transfection, conjugation, transduction, and the like. For stable transformation, a DNA sequence can further include a selectable marker, such as, antibiotic resistance. The selectable marker may provide antibiotic resistance to, for example, neomycin, tetracycline, chloramphenicol, or kanamycin. In addition, genes that complement resistance to auxotrophic deficiencies can be utilized.

In some embodiments, an expression vector that includes a heterologous DNA sequence encoding a protein involved in a metabolic or biosynthetic pathway is provided. Suitable expression vectors include, but are not limited to, viral vectors, such as baculovirus vectors, phage vectors, such as bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli, Pseudomonas pisum and Saccharomyces cerevisiae).

Useful expression vectors can include one or more selectable marker genes to provide a phenotypic trait for selection of transformed host cells. The selectable marker gene encodes a protein necessary for the survival or growth of transformed host cells grown in a selective culture medium. Host cells not transformed with the vector containing the selectable marker gene will not survive in the culture medium. Typical selection genes encode proteins that (a) confer resistance to antibiotics or other toxins (e.g., ampicillin, neomycin, methotrexate, or tetracycline) (b) complement auxotrophic deficiencies, or (c) supply critical nutrients not available from complex media (e.g., the gene encoding D-alanine racemase for Bacilli). In alternative embodiments of this invention, the selectable marker gene is one that encodes dihydrofolate reductase or confers neomycin resistance (for use in eukaryotic cell culture) or one that confers tetracycline or ampicillin resistance (for use in a prokaryotic host cell, such as E. coli).

The biosynthetic pathway gene product-encoding DNA sequence in the expression vector is operably linked to an appropriate expression control sequence, (promoters, enhancers, and the like) to direct synthesis of the encoded gene product. Such promoters can be derived from microbial or viral sources, including CMV and SV40. Depending on the host/vector system utilized, any number of suitable transcription and translation control elements, including constitutive and inducible promoters, transcription enhancer elements, transcription terminators, etc. can be used in the expression vector (see e.g., Bitter et al., Methods in Enzymology, 153:516-544, 1987).

Suitable promoters for use in prokaryotic host cells include, but are not limited to, promoters capable of recognizing the T4, T3, Sp6 and T7 polymerases, the P_{R} and P_{L} promoters of bacteriophage lambda, the trp, recA, heat shock, and lacZ promoters of E. coli, the α-amylase and the σ-specific promoters of B. subtilis, the promoters of the bacteriophages of Bacillus, Streptomyces promoters, the int promoter of bacteriophage lambda, the bla promoter of the β-lactamase gene of pBR322, and the CAT promoter of the chloramphenicol acetyl transferase gene. Prokaryotic promoters are reviewed by Glick, J. Ind. Microbiol. 1:277, 1987; Watson et al., MOLECULAR BIOLOGY OF THE GENE, 4th Ed., Benjamin Cummins (1987); and Sambrook et al., supra.

Non-limiting examples of suitable eukaryotic promoters for use within a eukaryotic host are viral in origin and include the promoter of the mouse metallothionein I gene (Hamer et al., J. Mol. Appl. Gen. 1:273, 1982); the TK promoter of Herpes virus (McKnight, Cell 31:355, 1982); the SV40 early promoter (Benoist et al., Nature (London) 290:304, 1981); the Rous sarcoma virus promoter; the cytomegalovirus promoter (Foecking et al., Gene 45:101, 1980); the yeast gal4 gene promoter (Johnston, et al., PNAS (USA) 79:6971, 1982; Silver, et al., PNAS (USA) 81:5951, 1984); and the IgG promoter (Orlandi et al., PNAS (USA) 86:3833, 1989).

The microbial host cell can be genetically modified with heterologous DNA sequence encoding a biosynthetic pathway gene product that is operably linked to an inducible promoter. Inducible promoters are well known in the art. Suitable inducible promoters include, but are not limited to, promoters that are affected by proteins, metabolites, or chemicals. These include: a bovine leukemia virus promoter, a metallothionein promoter, a dexamethasone-inducible MMTV promoter, a SV40 promoter, a MRP polIII promoter, a tetracycline-inducible CMV promoter (e.g., the human immediate-early CMV promoter) as well as those from the tip and lac operons.

In some examples, a genetically modified host cell is genetically modified with a heterologous DNA sequence encoding a biosynthetic pathway gene product that is operably linked to a constitutive promoter. Suitable constitutive promoters are known in the art and include constitutive adenovirus major late promoter, a constitutive MPSV promoter, and a constitutive CMV promoter.

In some examples a modified host cell is one that is genetically modified with an exogenous DNA sequence encoding a single protein involved in a biosynthesis pathway. In other embodiments, a modified host cell is one that is genetically modified with exogenous DNA sequences encoding two or more proteins involved in a biosynthesis pathway, for example, the first and second enzymes in a biosynthetic pathway.

Where the host cell is genetically modified to express two or more proteins involved in a biosynthetic pathway, those DNA sequences can each be contained in a single or in separate expression vectors. When those DNA sequences are contained in a single expression vector, in some embodiments, the nucleotide sequences will be operably linked to a common control element (e.g., a promoter) which controls expression of all of the biosynthetic pathway protein-encoding DNA sequences in the single expression vector.

When a modified host cell is genetically modified with heterologous DNA sequences encoding two or more proteins involved in a biosynthesis pathway, one of the DNA sequences can be operably linked to an inducible promoter, and one or more of the DNA sequences can be operably linked to a constitutive promoter.

In some embodiments, the intracellular concentration (e.g., the concentration of the intermediate in the genetically modified host cell) of the biosynthetic pathway intermediate can be increased to further boost the yield of the final product. The intracellular concentration of the intermediate can be increased in a number of ways, including, but not limited to, increasing the concentration in the culture medium of a substrate for a biosynthetic pathway; increasing the catalytic activity of an enzyme that is active in the biosynthetic pathway; increasing the intracellular amount of a substrate (e.g., a primary substrate) for an enzyme that is active in the biosynthetic pathway; and the like.

In some examples, the fatty ester, fatty. acid derivative, or intermediate is produced in the cytoplasm of the cell. The cytoplasmic concentration can be increased in a number of ways, including, but not limited to, binding of the fatty acid to coenzyme A to form an acyl-CoA thioester. Additionally, the concentration of these acyl-CoAs can be increased by increasing the biosynthesis of acyl-CoA in the cell, such as by over-expressing genes associated with pantothenate biosynthesis (panD) or knocking out the genes associated with glutathione biosynthesis (glutathione synthase).

Branching Including Cyclic Groups

Fatty esters and fatty acid derivatives can be produced that contain branch points, cyclic moieties, and combinations thereof, using the teachings provided herein. In some embodiments, microorganisms that naturally produce straight fatty acids (sFAs) can be engineered to produce branched chain fatty acids (brFAs) by expressing one or more exogenous nucleic acid sequences. For example, E. coli naturally produces straight fatty acids (sFAs). To engineer E. coli to produce brFAs, several genes can be introduced and expressed that provide branched precursors (bkd operon) and allow initiation of fatty acid biosynthesis from branched precursors (fabH). Additionally, the organism can express genes for the elongation of brFAs (e.g. ACP, FabF) and/or deleting the corresponding E. coli genes that normally lead to sFAs and would compete with the introduced genes (e.g. FabH, FabF).

The branched acyl-CoAs 2-methyl-buturyl-CoA, isovaleryl-CoA and isobuturyl-CoA are the precursors of brFA. In most brFA-containing microorganisms, they are synthesized in two steps (described in detail below) from branched amino acids (isoleucine, leucine and valine) (Kadena, Microbiol. Rev. 55: pp. 288, 1991). To engineer a microorganism to produce brFAs, or to overproduce brFAs, expression or over-expression of one or more of the enzymes in these two steps can be engineered. For example, in some instances the production host can have an endogenous enzyme that can accomplish one step and, therefore, only enzymes involved in the second step need to be expressed recombinantly.

The first step in forming branched fatty acids is the production of the corresponding α-keto acids by a branched-chain amino acid aminotransferase. E. coli has such an enzyme, IlvE (EC 2.6.1.42; Genbank accession YP_{—}026247). In some examples, a heterologous branched-chain amino acid aminotransferase may not be expressed. However, E. coli IlvE or any other branched-chain amino acid aminotransferase (e.g. ilvE from Lactococcus lactis (Genbank accession AAF34406), ilvE from Pseudomonas putida (Genbank accession NP 745648) or ilvE from Streptomyces coelicolor (Genbank accession NP_{—}629657)) can be over-expressed in a host microorganism if the aminotransferase reaction turns out to be rate limiting in brFA biosynthesis in the host organism chosen for fatty acid derivative production.

The second step, the oxidative decarboxylation of the α-ketoacids to the corresponding branched-chain acyl-CoA, is catalyzed by a branched-chain α-keto acid dehydrogenase complexes (bkd; EC 1.2.4.4.) (Denoya et al. J. Bacteriol. 177:pp. 3504, 1995), which consists of E1α/β (decarboxylase), E2 (dihydrolipoyl transacylase), and E3 (dihydrolipoyl dehydrogenase) subunits and are similar to pyruvate and α-ketoglutarate dehydrogenase complexes. Table 2 shows potential bkd genes from several microorganisms that can be expressed in a production host to provide branched-chain acyl-CoA precursors. Basically, every microorganism that possesses brFAs and/or grows on branched-chain amino acids can be used as a source to isolate bkd genes for expression in production hosts, for example, E. coli. Furthermore, E. coli has the E3 component (as part of its pyruvate dehydrogenase complex; 1pd, EC 1.8.1.4, Genbank accession NP_{—}414658). It can, therefore, only express the E1 α/β and E2 bkd genes.

TABLE 2
Bkd genes from selected microorganisms

| | | Genbank |
|---|---|---|
| Organism | Gene | Accession # |
| Streptomyces coelicolor | bkdA1 (E1α) | NP_628006 |
| | bkdB1 (E1β) | NP_628005 |
| | bkdC1 (E2) | NP_638004 |
| Streptomyces coelicolor | bkdA2 (E1α) | NP_733618 |
| | bkdB2 (E1β) | NP_628019 |
| | bkdC2 (E2) | NP_628018 |
| Streptomyces avermitilis | bkdA (E1a) | BAC72074 |
| | bkdB (E1b) | BAC72075 |
| | bkdC (E2) | BAC72076 |
| Streptomyces avermitilis | bkdF (E1α) | BAC72088 |
| | bkdG (E1β) | BAC72089 |
| | bkdH (E2) | BAC72090 |
| Bacillus subtilis | bkdAA (E1α) | NP_390288 |
| | bkdAB (E1β) | NP_390288 |
| | bkdB (E2) | NP_390288 |
| Pseudomonas putida | bkdA1 (E1α) | AAA65614 |
| | bkdA2 (E1β) | AAA65615 |
| | bkdC (E2) | AAA65617 |

In another example, isobuturyl-CoA can be made in a production host, for example, in E. coli through the coexpression of a crotonyl-CoA reductase (e.g., EC 1.1.1.9) and isobuturyl-CoA mutase (large subunit IcmA, EC 5.4.99.2; small subunit IcmB, EC 5.4.99.13) (Han and Reynolds J. Bacteriol. 179:pp. 5157, 1997). Crotonyl-CoA is an intermediate in fatty acid biosynthesis in E. coli and other microorganisms. Examples for ccr and icm genes from selected microorganisms are given in Table 3.

TABLE 3
Ccr and icm genes from selected microorganisms

| | | Genbank |
|---|---|---|
| Organism | Gene | Accession # |
| Streptomyces coelicolor | ccr | NP_630556 |
| | icmA | NP_629554 |
| | icmB | NP_630904 |
| Streptomyces cinnamonensis | ccr | AAD53915 |
| | icmA | AAC08713 |
| | icmB | AJ246005 |

In addition to expression of the bkd genes (see above), the initiation of brFA biosynthesis utilizes β-ketoacyl-acyl-carrier-protein synthase III (FabH, EC 2.3.1.41) with specificity for branched chain acyl-CoAs (Li et al. J. Bacteriol. 187:pp. 3795, 2005). Examples of such FabHs are listed in Table 4. fabH genes that are involved in fatty acid biosynthesis of any brFA-containing microorganism can be expressed in a production host. The Bkd and FabH enzymes from production hosts that do not naturally make brFA may not support brFA production and, therefore, bkd and fabH can be expressed recombinantly. Similarly, the endogenous level of Bkd and FabH production may not be sufficient to produce brFA. Therefore, they can be over-expressed. Additionally, other components of fatty acid biosynthesis machinery can be expressed, such as acyl carrier proteins (ACPs) and β-ketoacyl-acyl-carrier-protein synthase II (fabF, EC 2.3.1.41) (candidates are listed in Table 4). In addition to expressing these genes, some genes in the endogenous fatty acid biosynthesis pathway can be attenuated in the production host. For example, in E. coli the most likely candidates to interfere with brFA biosynthesis are fabH (Genbank accession #NP_{—}415609) and/or fabF genes (Genbank accession #NP_{—}415613).

As mentioned above, through the combination of expressing genes that support brFA synthesis and alcohol synthesis branched chain alcohols can be produced. For example, when an alcohol reductase, such as Acr1 from Acinetobacter baylyi ADP1 is coexpressed with a bkd operon, E. coli can synthesize isopentanol, isobutanol, or 2-methyl butanol. Similarly, when Acr1 is coexpressed with ccr/icm genes, E. coli can synthesize isobutanol.

In order to convert a production host, such as E. coli, into an organism capable of synthesizing ω-cyclic fatty acids (cyFAs), several genes can be introduced and expressed that provide the cyclic precursor cyclohexylcarbonyl-CoA (Cropp et al. Nature Biotech. 18:pp. 980, 2000). One or more of the genes listed in Table 4 (e.g., fabH, ACP, and fabF) can be expressed to allow initiation and elongation of ω-cyclic fatty acids. Alternatively, the homologous genes can be isolated from microorganisms that make cyFAs and expressed in E. coli.

TABLE 4
FabH, ACP and fabF genes from selected microorganisms with brFAs

| | | Genbank |
|---|---|---|
| Organism | Gene | Accession # |
| Streptomyces coelicolor | fabH1 | NP_626634 |
| | ACP | NP_626635 |
| | fabF | NP_626636 |
| Streptomyces avermitilis | fabH3 | NP_823466 |
| | fabC3 (ACP) | NP_823467 |
| | fabF | NP_823468 |
| Bacillus subtilis | fabH_A | NP_389015 |
| | fabH_B | NP_388898 |
| | ACP | NP_389474 |
| | fabF | NP_389016 |
| Stenotrophomonas | SmalDRAFT_0818 (FabH) | ZP_01643059 |
| maltophilia | SmalDRAFT_0821 (ACP) | ZP_01643063 |
| | SmalDRAFT_0822 (FabF) | ZP_01643064 |
| Legionella pneumophila | FabH | YP_123672 |
| | ACP | YP_123675 |
| | fabF | YP_123676 |

Expression of the following genes are sufficient to provide cyclohexylcarbonyl-CoA in E. coli: ansJ, ansK, ansL, chcA, and ansM from the ansatrienin gene cluster of Streptomyces collinus (Chen et al., Eur. J. Biochem. 261:pp. 1999, 1999) or plmJ, plmK, plmL, chcA, and plmM from the phoslactomycin B gene cluster of Streptomyces sp. HK803 (Palaniappan et al., J. Biol. Chem. 278:pp. 35552, 2003) together with the chcB gene (Patton et al. Biochem., 39:pp. 7595, 2000) from S. collinus, S. avermitilis, or S. coelicolor (see Table 5 for Genbank accession numbers).

TABLE 5
Genes for the synthesis of cyclohexylcarbonyl-CoA

| Organism | Gene | Genbank Accession # |
|---|---|---|
| Streptomyces collinus | ansJK | U72144* |
| | ansL | |
| | chcA | |
| | ansL | |
| | chcB | AF268489 |
| Streptomyces sp. HK803 | pmlJK | AAQ84158 |
| | pmlL | AAQ84159 |
| | chcA | AAQ84160 |
| | pmlM | AAQ84161 |
| Streptomyces coelicolor | chcB/caiD | NP_629292 |
| Strepromyces avermitilis | chcB/caiD | NP_629292 |

Only chcA is annotated in Genbank entry U72144, ansJKLM are according to Chen et al. (Eur. J. Biochem. 261: pp. 1999, 1999)

The genes listed in Table 4 (fabH, ACP and fabF) are sufficient to allow initiation and elongation of ω-cyclic fatty acids because they can have broad substrate specificity. In the event that coexpression of any of these genes with the ansJKLM/chcAB or pmlJKLM/chcAB genes from Table 5 does not yield cyFAs, fabH, ACP, and/or fabF homologs from microorganisms that make cyFAs can be isolated (e.g., by using degenerate PCR primers or heterologous DNA probes) and coexpressed. Table 6 lists selected microorganisms that contain ω-cyclic fatty acids.

TABLE 6
Examples of microorganisms that contain ω-cyclic fatty acids

| Organism | Reference |
|---|---|
| Curtobacterium pusillum | ATCC19096 |
| Alicyclobacillus acidoterrestris | ATCC49025 |
| Alicyclobacillus acidocaldarius | ATCC27009 |
| Alicyclobacillus cycloheptanicum* | Moore, J. Org. Chem. 62: |
| | pp. 2173, 1997. |

*uses cycloheptylcarbonyl-CoA and not cyclohexylcarbonyl-CoA as precursor for cyFA biosynthesis

As will be appreciated by one of skill in the art, any one or combination of the products discussed above can be incorporated into the fatty esters discussed herein.

Saturation

Production hosts can be engineered to produce unsaturated fatty acids by engineering the production host to over-express fabB or by growing the production host at low temperatures (e.g., less than 37° C.). FabB has a preference for cis-δ³decenoyl-ACP and results in unsaturated fatty acid production in E. coli. Over-expression of fabB resulted in the production of a significant percentage of unsaturated fatty acids (de Mendoza et al., J. Biol. Chem., 258:2098-101, 1983). These unsaturated fatty acids can then be used as intermediates in production hosts that are engineered to produce fatty acid derivatives, such as fatty alcohols, esters, waxes, olefins, alkanes, and the like. One of ordinary skill in the art will appreciate that by attenuating fabA or over-expressing fabB and expressing specific thioesterases (described below), unsaturated fatty acid derivatives having a desired carbon chain length can be produced. Alternatively, the repressor of fatty acid biosynthesis, fabR (Genbank accession NP_{—}418398), can be deleted, which will also result in increased unsaturated fatty acid production in E. coli (Zhang et al., J. Biol. Chem. 277:pp. 15558, 2002). Further increases in unsaturated fatty acids can be achieved by over-expression of fabM (trans-2, cis-3-decenoyl-ACP isomerase, Genbank accession DAA05501) and controlled expression of fabK (trans-2-enoyl-ACP reductase II, Genbank accession NP_{—}357969) from Streptococcus pneumoniae (Marrakchi et al., J. Biol. Chem. 277: 44809, 2002), while deleting E. coli fab I ((trans-2-enoyl-ACP reductase, Genbank accession NP_{—}415804). Additionally, to increase the percentage of unsaturated fatty esters, the microorganism can also have fabB (encoding β-ketoacyl-ACP synthase I, Accessions: BAA16180, EC:2.3.1.41), sfa (encoding a suppressor of fabA, Accession: AAC44390), or gnsA and gnsB (both encoding SecG null mutant suppressors (i.e., cold shock proteins), Accession: ABD18647.1, AAC74076.1) over-expressed.

In some examples, the endogenous fabF gene can be attenuated. This will increase the percentage of palmitoleate (C16:1) produced.

Exemplary Production Hosts

A production host, including those for fatty ester production, can include plant, animal, human, bacteria, yeast, or filamentous fungi cells. Additional production hosts include the following: a mammalian cell, plant cell, insect cell, yeast cell, fungus cell, filamentous fungi cell, bacterial cell, a Gram-positive bacteria, a Gram-negative bacteria, the genus Escherichia, the genus Bacillus, the genus Lactobacillus, the genus Rhodococcus, the genus Pseudomonas, the genus Aspergillus, the genus Trichoderma, the genus Neurospora, the genus Fusarium, the genus Humicola, the genus Rhizomucor, the genus Kluyveromyces, the genus Pichia, the genus Mucor, the genus Myceliophtora, the genus Penicillium, the genus Phanerochaete, the genus Pleurotus, the genus Trametes, the genus Chrysosporium, the genus Saccharomyces, the genus Stenotrophamonas, the genus Schizosaccharomyces, the genus Yarrowia, the genus Streptomyces, a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, a Mucor michei cell, a Streptomyces lividans cell, a Streptomyces murinus cell, an Actinomycetes cell, a CHO cell, a COS cell, a VERO cell, a BHK cell, a HeLa cell, a Cv1 cell, an MDCK cell, a 293 cell, a 3T3 cell, a PC12 cell, an E. coli cell, a strain B E. coli cell, a strain C E. coli cell, a strain K E. coli cell, and a strain W E. coli cell. Additional production hosts can be selected from the group consisting of Bacteria: (g-positive) Bacillus (B. lentus, B. brevis, B. stearothermophilus, B. licheniformis, B. alkalophilus, B. coagulans, B. circulans, B pumilis, B. thuringiensis, B. clausii, B. megaterium, B. subtilis, B. amyloliquefaciens), Lactobacillus; Bacteria: (g-negative): pseudomonas; Filamentous Fungi: Trichoderma (koningii, viride, reesei, longibrachiatum), Aspergillus (awamori, fumigatis, foetidus, nidulans, niger, oryzae), Fusarium, Humicola (Humicola insolens, Humicola lanuginosa), Rhizomucor (R. miehei), Kluyveromyces, Pichia, Mucor (michei), Neurospora, Myceliophtora, Penicillium, Phanerochaete, Pleurotus, Trametes; Yeast: Saccharomyces, Schizosaccharomyces, Yarrowia; Actinomycetes, e.g., streptomyces (Streptomyces lividans or Streptomyces murinus), and CHO cells.

In some embodiments, one or more production hosts are present in a production vessel. In some embodiments, one or more production hosts are used to make the same product (e.g., ethanol or fatty esters). In some embodiments, 1, 2, 3, 4, 5, 6, 7, 8, 9, 10, 11, 12, 13, 14, 15, 16, 17, 18, 19, 20 or more types of production hosts are together. In some embodiments, the production host is isolated from other production hosts.

For ethanol production, examples of suitable production hosts include yeast, bacteria, Saccharomyces cerevisiae, Saccharomyces distaticus, Saccharomyces uvarum, Schizosaccharomyces pombe, Kluyveromyces marxianus, Kluyveromyces fragilis, Candida pseudotropicalis, Candida brassicae, Clostridium acetobutylicum, Clavispora lusitaniae, Clavispora opuntiae, Pachysolen tannophilus, Bretannomyces clausenii, Zymomonas mobilis, Clostridium thermocellum, and various strains of Escherichia coli, including those described in paragraphs 98-99 of U.S. Patent Publication US2002/0137154 (incorporated herein by reference). Ethanol production hosts also include Klebsiella oxytoca strains, including those described in paragraphs 100-101 of U.S. Patent Publication US2002/0137154 (incorporated herein by reference), as well as the microorganisms described in paragraphs 26-29 of U.S. Patent Publication 2003/0054500 (incorporated herein by reference). Further examples of suitable production hosts for producing ethanol are recombinant bacteria strains, such as B. subtillis, described in U.S. Patent Publication US2005/0158836. Further examples of suitable production hosts for producing ethanol are described in U.S. Pat. No. 7,205,138, which describes methods of producing a product having between 5 to 20% ethanol using a granular starch production substrate, an acid-stable alpha amylase having granular starch hydrolyzing activity, a glucoamylase, and an ethanol producing microorganism, such as yeasts, including strains of Saccharomyces, such as S. cerevisiae. Other suitable production hosts are described in Linden, Industrially Important Strains and Pathways in Handbook of Anaerobic Fermentations, 1988, pp. 59-80; Nakashima, Progress in Ethanol Production With Yeasts, Yeasts, Biotechnology, and Biocatalysis 1990, p 57-84, Benitez et al, Production of Ethanol By Yeast, Handbook of Applied Mycology 4 Fungal Biotechnology 1992, pp. 603-680, and Lida, Fuel Ethanol Production By Immobilized Yeasts and Yeast Immobilization, Industrial Application of Immobilized Biocatalysts, 1993 pp. 163-182 (the entireties of each of which is incorporated by reference).

As noted herein, in some embodiments, the production host can produce short chain alcohols, such as ethanol, propanol, isopropanol, isobutanol, and butanol for incorporation in A₁ using techniques well known in the art. For example, butanol can be made by the host organism. To create butanol producing cells, the E. coli C41 (DE3, ΔfadE) LS9001 strain (described in Example 1, below) can be further engineered to produce A to B (acetyl-CoA acetyltransferase) from Escherichia coli K12, β-hydroxybutyryl-CoA dehydrogenase from Butyrivibrio fibrisolvens, crotonase from Clostridium beijerinckii, butyryl CoA dehydrogenase from Clostridium beijerinckii, CoA-acylating aldehyde dehydrogenase (ALDH) from Cladosporium fulvum, and AdhE (aldehyde-alcohol dehydrogenase) of Clostridium acetobutylicum in the pBAD24 expression vector under the prpBCDE promoter system. Similarly, ethanol can be produced in a production host using the methods taught by Kalscheuer et al., Microbiology 152:2529-2536, 2006, which is herein incorporated by reference. In some embodiments, a single production host makes both the fatty ester and the alcohol. In some embodiments, two different hosts are responsible for processing the fatty ester and the alcohol.

### Fatty Esters

Production hosts can be engineered using known peptides to produce fatty esters from acyl-CoA and alcohols. In some examples the alcohols are provided in the production media, and in other examples the production host can provide the alcohol as described herein.

One of ordinary skill in the art will appreciate that structurally, fatty esters have an A and a B side (or group). In some embodiments, the fatty ester comprises, consists, or consists essentially of the following formula:

B₁COOA₁

B₁ is an aliphatic group. In some embodiments, B₁ is a carbon chain. In some embodiments it is at least 5, 6, 7, 8, 9, 10, 11, 12, 13, 14, 15, 16, 17, 18, 19, 20, 21, 22, 23, 24, 25, 26, 27, 28, 29, or 30 carbons in length. In some embodiments, B₁ is an aliphatic group (e.g., an alkyl group). A₁ will include at least one carbon. In some embodiments, A₁ is an aliphatic group. In some embodiments, A₁ is an alkyl group. In some embodiments, the alkyl group comprises, consists, or consists essentially of 1, 2, 3, 4, 5, 6, 7, 8, 9, 10, 11, 12, 13, 14, 15, 16, 17, 18, 19, or 20 carbon atoms. In some embodiments, any of the above B₁ groups can be combined with any of the above A₁ groups. In some embodiments, A₁ comprises, consists, or consists essentially of a carbon chain having a number of carbons selected from the group consisting of 1, 2, 3, 4, and 5 carbon atoms while B₁ is a carbon chain that comprises, consists, or consists essentially of at least 12, 13, 14, 15, 16, 17, 18, 19, or 20 carbons.

In some embodiments, fatty esters are esters derived from a fatty acyl-thioester and an alcohol, wherein the A side and the B side of the fatty ester can vary in length independently. In some embodiments, the A side of the fatty ester is at least 1, 2, 3, 4, 5, 6, 7, or 8 carbons in length, while the B side of the fatty ester can be any useable length, for example, 6, 7, 8, 9, 10, 11, 12, 13, 14, 15, 16, 17, 18, 19, 20, 21, 22, 23, 24, 25, or 26 carbons in length. The A side and the B side can be straight chain or branched, saturated or unsaturated.

The production of fatty esters, including waxes, from acyl-CoA and alcohols can be engineered using known polypeptides. As used herein, waxes are long chain fatty esters, wherein the A side and the B side of the fatty ester can vary in length independently. Generally, the A side of the fatty ester is at least 1, 2, 3, 4, 5, 6, 7, 8, 9, 10, 11, 12, 13, 14, 15, 16, 17, 18, 19, 20, 21, 22, 23, 24, 25, or 26 carbons in length. Similarly the B side of the fatty ester is at least 6, 7, 8, 9, 10, 11, 12, 13, 14, 15, 16, 17, 18, 19, 20, 21, 22, 23, 24, 25, or 26 carbons in length. The A side and the B side can be mono-, di-, tri-unsaturated. The production of fatty esters, including waxes, from acyl-CoA and alcohols can be engineered using known polypeptides. One method of making fatty esters includes increasing the expression of or expressing more active forms of one or more ester synthases (EC 2.3.1.75).

When discussed in reference to the addition of an alcohol to acyl-CoA, the A side of the fatty ester is used to describe the carbon chain contributed by the alcohol and the B side of the fatty ester is used to describe the carbon chain contributed by the acyl-CoA.

In some embodiments, A₁ and/or B₁ are saturated or unsaturated, branched or unbranched, or any combination thereof. In some embodiments, the B side is saturated. In some embodiments, the B side is unsaturated. In some embodiments, B₁ has a single unsaturated bond. In some embodiments, B₁ is polyunsaturated. In some embodiments, A₁ is saturated. In some embodiments, A₁ is unsaturated. In some embodiments, A₁ has a single unsaturated bond. In some embodiments, A₁ is polyunsaturated. In some embodiments, A₁ and B₁ can be mono-, di-, or tri-unsaturated simultaneously or independently. In some embodiments, any of the previous A₁ and B₁ options can be combined with each other, in any combination.

In some embodiments, the methods of described herein permit production of fatty esters of varied length. In some examples, the fatty ester product is a saturated or unsaturated fatty ester product having a carbon atom content limited to between 24 and 46 carbon atoms. In one embodiment, the fatty ester product has a carbon atom content limited to between 24 and 32 carbon atoms. In another embodiment, the fatty ester product has a carbon content of 14 and 20 carbons. In another embodiment, the fatty ester is the methyl ester of C18:1 (or “C_{18:1}” in which “18” denotes the number of carbons present and “1” denotes the number of double bonds). In another embodiment, the fatty ester is the ethyl ester of C16:1. In another embodiment, the fatty ester is the methyl ester of C16:1. In another embodiment, the fatty ester is octadecyl ester of octanol. In another embodiment, the product is a mixture of fatty esters in which greater than about 50%, or greater than about 60%, or greater than about 70%, or greater than about 80%, or greater than about 90% by volume of the component fatty esters have a melting point below about 4 degrees Celsius, below about 0 degrees Celsius, below about −10 degrees Celsius, or below about −20 degrees Celsius.

In some embodiments, B₁ can have a double bond at one or more points in the carbon chain. Thus, in some embodiments, a 6, 7, 8, 9, 10, 11, 12, 13, 14, 15, 16, 17, 18, 19, 20, 21, 22, 23, 24, 25, 26, 27, 28, 29, or 30 carbon long chain can have 1, 2, 3, 4, 5, 6, 7, 8, 9, 10, 11, 12, 13, 14, 15, 16, 17, 18, 19, 20, 21, 22, 23, or 24 double bonds and 1-24 of those double bonds can be located following carbon 1, 2, 3, 4, 5, 6 7, 8, 9, 10, 11, 12, 13, 14, 15, 16, 17, 18, 19, 20, 21, 22, 23, 24, 25, 26, 27, 28, or 29. In some embodiments, a 1, 2, 3, 4, 5, or 6 carbon chain for A₁ can have 1, 2, 3, 4, or 5 double bonds and 1-5 of those double bonds can be located following carbon 1, 2, 3, 4, or 5. In some embodiments, any of the above A₁ groups can be combined with any of the above B₁ groups.

The production host can be engineered to produce fatty alcohols or short chain alcohols. The production host can also be engineered to produce specific acyl-CoA molecules.

In some embodiments, B₁ is contributed by a fatty acid produced from de novo synthesis in the host organism. In some embodiments, where the host is additionally engineered to make alcohols, including fatty alcohols, A₁ is also produced by the host organism. In some embodiments, the A₁ side can be provided in the medium. As described herein, by selecting the desired thioesterase genes, B₁ can be designed to have certain carbon chain characteristics. These characteristics include points of unsaturation, branching, and desired carbon chain lengths. For example, at least about 50%, 60%, 70%, 80%, 85%, 90%, 95%, 98%, 99% by volume of the fatty esters produced will have A, and B₁ that vary by 6, 4, or 2 carbons in length. In some embodiments, A₁ and B₁ will also display similar branching and saturation levels. In some embodiments, at least about 50%, 50-60%, 60-70%, 70-80%, 80-90%, 90-95%, 95-98%, 98-99%, or greater percent of the fatty esters produced will have A₁ and B₁ that vary by 6, 4, or 2 carbons in length.

Carbon Chain Characteristics

In some embodiments, the hydrocarbons, fatty alcohols, fatty esters, and waxes disclosed herein are useful as biofuels and specialty chemicals. The products can be produced such that they contain desired branch points, levels of saturation, and carbon chain length. Therefore, these products can be desirable starting materials for use in many applications (FIG. 9 provides a description of the various enzymes that can be used alone or in combination to make various fatty acid derivatives). FIG. 9 also identifies various genes that can be modulated to alter the structure of the fatty acid derivative product. One of ordinary skill in the art will appreciate that some of the genes that are used to alter the structure of the fatty acid derivative can also increase the production of fatty acid derivatives.

Furthermore, biologically produced fatty acid derivatives (including fatty esters) represent a new feedstock for fuels, such as alcohols, diesel and gasoline. Fatty esters and some biofuels made using fatty acid derivatives have not been produced from renewable sources and, as such, are new compositions of matter. These new fatty esters and fuels can be distinguished from fatty esters and fuels derived from petrochemical carbon on the basis of dual carbon-isotopic fingerprinting. Additionally, the specific source of biosourced carbon (e.g. glucose vs. glycerol) can be determined by dual carbon-isotopic fingerprinting (see, U.S. Pat. No. 7,169,588, which is herein incorporated by reference). The following discussion generally outlines two options for distinguishing chemically-identical materials (that have the same structure, but different isotopes). In some embodiments, this apportions carbon in products by source (and possibly year) of growth of the biospheric (plant) component.

The isotopes, ¹⁴C and ¹³C, bring complementary information to this examination. The radiocarbon dating isotope (¹⁴C), with its nuclear half life of 5730 years, clearly allows one to apportion specimen carbon between fossil (“dead”) and biospheric (“alive”) feedstocks [Currie, L. A. “Source Apportionment of Atmospheric Particles,” Characterization of Environmental Particles, J. Buffle and H. P. van Leeuwen, Eds., 1 of Vol. 1 of the IUPAC Environmental Analytical Chemistry Series (Lewis Publishers, Inc) (1992) 3 74]. The basic understanding in radiocarbon dating is that the constancy of ¹⁴C concentration in the atmosphere leads to the constancy of ¹⁴C in living organisms. When dealing with an isolated sample, the age of a sample can be deduced approximately by the relationship t=(−5730/0.693)ln(A/A₀) (Equation 1) where t=age, 5730 years is the half-life of radiocarbon, and A and A₀ are the specific ¹⁴C activity of the sample and of the modern standard, respectively [Hsieh, Y., Soil Sci. Soc. Am J., 56, 460, (1992)]. However, because of atmospheric nuclear testing since 1950 and the burning of fossil fuel since 1850, ¹⁴C has acquired a second, geochemical time characteristic. Its concentration in atmospheric CO₂, and hence in the living biosphere, approximately doubled at the peak of nuclear testing, in the mid-1960s. It has since been gradually returning to the steady-state cosmogenic (atmospheric) baseline isotope rate (¹⁴C/¹²C) of ca. 1.2×10¹² with an approximate relaxation “half-life” of 7-10 years. (This latter half-life must not be taken literally; rather, one must use the detailed atmospheric nuclear input/decay function to trace the variation of atmospheric and biospheric ¹⁴C since the onset of the nuclear age.) It is this latter biospheric ¹⁴C time characteristic that holds out the promise of annual dating of recent biospheric carbon. ¹⁴C can be measured by accelerator mass spectrometry (AMS) with results given in units of “fraction of modern carbon” (f_{M}). f_{M} is defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

The stable carbon isotope ratio (¹³C/¹²C) provides a complementary route to source discrimination and apportionment. The ¹³C/¹²C ratio in a given biosourced material is a consequence of the ¹³C/¹²C ratio in atmospheric carbon dioxide at the time the carbon dioxide is fixed and also reflects the precise metabolic pathway. Regional variations also occur. Petroleum, C3 plants (the broadleaf), C₄ plants (the grasses), and marine carbonates all show significant differences in ¹³C/¹²C and their corresponding delta¹³C values. Furthermore, lipid matter of C3 and C4 plants analyze differently than materials derived from the carbohydrate components of the same plants as a consequence of the metabolic pathway. Within the precision of measurement, ¹³C shows large variations due to isotopic fractionation effects, the most significant of which for the instant invention is the photosynthetic mechanism. The major cause of differences in the carbon isotope ratio in plants is closely associated with differences in the pathway of photosynthetic carbon metabolism in the plants, particularly the reaction occurring during the primary carboxylation (i.e., the initial fixation of atmospheric CO₂). Two large classes of vegetation are those that incorporate the “C3” (or Calvin-Benson) photosynthetic cycle and those that incorporate the “C4” (or Hatch-Slack) photosynthetic cycle. C3 plants, such as hardwoods and conifers, are dominant in the temperate climate zones. In C3 plants, the primary CO₂ fixation or carboxylation reaction involves the enzyme ribulose-1,5-diphosphate carboxylase and the first stable product is a 3-carbon compound. C4 plants, on the other hand, include such plants as tropical grasses, corn and sugar cane. In C4 plants, an additional carboxylation reaction involving another enzyme, phosphoenol-pyruvate carboxylase, is the primary carboxylation reaction. The first stable carbon compound is a 4-carbon acid which is subsequently decarboxylated. The CO₂ thus released is refixed by the C3 cycle.

Both C4 and C3 plants exhibit a range of ¹³C/¹²C isotopic ratios, but typical values are about −10 to −14 per mil (C4) and −21 to −26 per mil (C3) [Weber et al., J. Agric. Food Chem., 45, 2942 (1997)]. Coal and petroleum fall generally in this latter range. The ¹³C measurement scale was originally defined by a zero set by pee dee belemnite (PDB) limestone, where values are given in parts per thousand deviations from this material. The “δ¹³”, values are in parts per thousand (per mil), abbreviated ‰, and are calculated as follows:

$${δ}^{13} C = \frac{{( {}^{13} C / {}^{12} C )}_{sample} - {( {}^{13} C / {}^{12} C )}_{standard}}{{( {}^{13} C / {}^{12} C )}_{standard}} \times 1000$$

Since the PDB reference material (RM) has been exhausted, a series of alternative RMs have been developed in cooperation with the IAEA, USGS, NIST, and other selected international isotope laboratories. Notations for the per mil deviations from PDB is δ¹³. Measurements are made on CO₂ by high precision stable ratio mass spectrometry (IRMS) on molecular ions of masses 44, 45, and 46.

The fatty acid derivatives, fatty esters, and the associated biofuels, chemicals, and mixtures can be distinguished from their petrochemical derived counterparts on the basis of ¹⁴C (fM) and dual carbon-isotopic fingerprinting, indicating new compositions of matter.

In some embodiments, the fatty acid derivatives and fatty esters described herein have utility in the production of biofuels and chemicals. The new fatty acid derivative or fatty ester based product compositions provided herein additionally can be distinguished on the basis of dual carbon-isotopic fingerprinting from those materials derived solely from petrochemical sources. The ability to distinguish these products is beneficial in tracking these materials in commerce. For example, fuels or chemicals comprising both “new” and “old” carbon isotope profiles can be distinguished from fuels and chemicals made only of “old” materials. Hence, the instant materials can be followed in commerce on the basis of their unique profile.

In some examples, a biofuel composition is made that includes a fatty id derivative or and fatty ester having δ¹³ of from about −10.9 to about −15.4, wherein the fatty acid derivative or fatty ester accounts for at least about 85% by volume of biosourced material (derived from a renewable resource such as cellulosic materials and sugars) in the composition. In other examples, the biofuel composition includes a fatty acid derivative or fatty ester having the formula

X—(CH(R))ₙCH₃

wherein X represents CH₃, —CH₂OR¹; —C(O)OR²; or —C(O)NR³R⁴;

R is, for each n, independently absent, H or lower aliphatic;

n is an integer from 8 to 34, such as from 10 to 24; and

R¹, R², R³ and R⁴ independently are selected from H and lower aliphatic. Typically, when R is lower aliphatic, R represents a branched, unbranched or cyclic lower alkyl or lower alkenyl moiety. Exemplary R groups include, without limitation, methyl, isopropyl, isobutyl, sec-butyl, cyclopentenyl, and the like.

In some embodiments, the fatty acid derivative is additionally characterized as having a δ¹³ of from about −10.9 to about −15.4; and the fatty acid derivative accounts for at least about 85% by volume of biosourced material in the composition. In some examples, the fatty acid derivative in the biofuel composition is characterized by having a fraction of modern carbon (f_{M} ¹⁴C) of at least about 1.003, 1.010, or 1.5.

### Processing

In some embodiments, the production and isolation of fatty acid derivatives or fatty esters can be enhanced by employing specific processing techniques. One method for increasing production while reducing costs is increasing the percentage of the carbon source that is converted to hydrocarbon products. During normal cellular lifecycles, carbon is used in cellular functions including producing lipids, saccharides, proteins, organic acids, and nucleic acids. Reducing the amount of carbon necessary for growth-related activities can increase the efficiency of carbon source conversion to output. This can be achieved by first growing microorganisms to a desired density, such as a density achieved at the peak of the log phase of growth. At such a point, replication checkpoint genes can be harnessed to stop the growth of cells. Specifically, quorum sensing mechanisms (reviewed in Camilli and Bassler Science 311:1113, 2006; Venturi FEMS Microbio Rev 30:274-291, 2006; and Reading and Sperandio FEMS Microbiol Lett 254:1-11, 2006) can be used to activate genes such as p53, p21, or other checkpoint genes. Genes that can be activated to stop cell replication and growth in E. coli include umuDC genes, the over-expression of which stops the progression from stationary phase to exponential growth (Murli et al., J. of Bact. 182:1127, 2000). UmuC is a DNA polymerase that can carry out translesion synthesis over non-coding lesions, the mechanistic basis of most UV and chemical mutagenesis. The umuDC gene products are used for the process of translesion synthesis and also serve as a DNA damage checkpoint. umuDC gene products include UmuC, UmuD, umuD′, UmuD′₂C, UmuD′₂, and UmuD₂. Simultaneously, the product producing genes would be activated, thus minimizing the need for replication and maintenance pathways to be used while the fatty acid derivative is being made.

The percentage of input carbons converted to hydrocarbon products is a cost driver. The more efficient (i.e., the higher the percentage) the conversion is, the less expensive the process will be. For oxygen-containing carbon sources (i.e. glucose and other carbohydrate based sources), the oxygen must be released in the form of carbon dioxide. For every 2 oxygen atoms released, a carbon atom is also released leading to a maximal theoretical metabolic efficiency of ˜34% (w/w) (for fatty acid derived products). This figure, however, changes for other hydrocarbon products and carbon sources. Typical efficiencies in the literature are less than about 5%. Engineered microorganisms which produce hydrocarbon products can have greater than about 1, 3, 5, 10, 15, 20, 25, and 30% efficiency. In some embodiments, microorganisms will exhibit an efficiency of about 10% to about 25%. In other embodiments, such microorganisms will exhibit an efficiency of about 25% to about 30%, and in other examples such microorganisms will exhibit greater than about 30% efficiency.

In some embodiments, where the final product is released from the cell, a continuous process can be employed. In this approach, a reactor with organisms producing fatty acid derivatives can be assembled in multiple ways. In one example, a portion of the media is removed and let to sit. Fatty acid derivatives are separated from the aqueous layer, which will in turn, be returned to the fermentation chamber.

In one example, the fermentation chamber will enclose a fermentation that is undergoing a continuous reduction. In this instance, a stable reductive environment would be created. The electron balance would be maintained by the release of carbon dioxide (in gaseous form). Efforts to augment the NAD/H and NADP/H balance can also facilitate in stabilizing the electron balance.

The availability of intracellular NADPH can also be enhanced by engineering the production host to express an NADH:NADPH transhydrogenase. The expression of one or more NADH:NADPH transhydrogenases converts the NADH produced in glycolysis to NADPH which enhances the production of fatty acid derivatives.

Disclosed herein is a system for continuously producing and exporting fatty acid derivatives out of recombinant host microorganisms via a transport protein. Many transport and efflux proteins serve to excrete a large variety of compounds and can be evolved to be selective for a particular type of fatty acid derivatives. Thus, in some embodiments an exogenous DNA sequence encoding an ABC transporter will be functionally expressed by the recombinant host microorganism so that the microorganism exports the fatty acid derivative into the culture medium. In one example, the ABC transporter is an ABC transporter from Caenorhabditis elegans, Arabidopsis thalania, Alkaligenes eutrophus, or Rhodococcus erythropolis (locus AAN73268). In another example, the ABC transporter is an ABC transporter chosen from CER5 (locuses At1g51500 or AY734542), AtMRP5, AmiS2, and AtPGP1. In some examples, the ABC transporter is CER5. In yet another example, the CER5 gene is from Arabidopsis (locuses At1g51500, AY734542, At3g21090 and At1g51460).

The transport protein, for example, can also be an efflux protein selected from: AcrAB, TolC, and AcrEF from E. coli, or Tll1618, Tll1619, and Tll10139 from Thermosynechococcus elongatus BP-1.

In addition, the transport protein can be, for example, a fatty acid transport protein (FATP) selected from Drosophila melanogaster, Caenorhabditis elegans, Mycobacterium tuberculosis, or Saccharomyces cerevisiae or any one of the mammalian FATP's. [0316] Production hosts can also be chosen for their endogenous ability to release fatty acid derivatives. The efficiency of product production and release into the fermentation broth can be expressed as a ratio of intracellular product to extracellular product. In some examples, the ratio can be 5:1, 4:1, 3:1, 2:1, 1:1, 1:2, 1:3, 1:4, or 1:5.

The production host can be additionally engineered to express recombinant cellulosomes, such as those described in PCT application number PCT/US2007/003736, which will allow the production host to use cellulosic material as a carbon source. For example, the production host can be additionally engineered to express invertases (EC 3.2.1.26) so that sucrose can be used as a carbon source.

Similarly, the production host can be engineered using the teachings described in U.S. Pat. Nos. 5,000,000, 5,028,539, 5,424,202, 5,482,846, and 5,602,030 to Ingram et al. so that the production host can assimilate carbon efficiently and use cellulosic materials as carbons sources.

### Processing Conditions

As will be appreciated by one of skill in the art, the conditions for allowing a production host to process a production substrate into a desired product (e.g., a fatty ester or an alcohol) will vary based upon the specific production host. In some embodiments, the process occurs in an aerobic environment. In some embodiments, the process occurs in an anaerobic environment. In some embodiments, the process occurs in a micro-aerobic environment.

In some embodiments, the amount of production host, production substrate, and ethanol in a fatty ester production process is between about 25 mg/L to about 2 g/L production host, between about 50 g/L and about 200 g/L production substrate, and about 10 mL/L to about 1000 mL/L ethanol, such as between about 75 mL/L and about 250 mg/L production host, about 150 mg/L to about 500 mg/L glucose, and about 25 mL/L to about 100 mL/L ethanol.

In some embodiments, cells (e.g., production hosts) are not added during the production process. In some embodiments, the alcohol composition is added to the fatty ester production host incrementally. In some embodiments, alcohol can be trapped from fatty ester production vessel off gas and be recycled back to the fatty ester production vessel.

### Post Production Processing

The fatty acid derivatives or fatty esters produced during production can be separated from the production media. Any technique known for separating fatty acid derivatives or fatty esters from aqueous media can be used. One exemplary separation process provided herein is a two phase (bi-phasic) separation process. This process involves processing the genetically engineered production hosts under conditions sufficient to produce a fatty acid derivative (e.g., a fatty ester), allowing the derivative to collect in an organic phase and separating the organic phase from the aqueous production broth. This method can be practiced in both a batch and continuous production setting.

Bi-phasic separation uses the relative immisiciblity of fatty acid derivatives to facilitate separation. Immiscible refers to the relative inability of a compound to dissolve in water and is defined by the compounds partition coefficient. The partition coefficient, P, is defined as the equilibrium concentration of compound in an organic phase (in a bi-phasic system the organic phase is usually the phase formed by the fatty acid derivative during the production process. However, in some examples an organic phase can be provided (e.g., a layer of octane to facilitate product separation)) divided by the concentration at equilibrium in an aqueous phase (i.e., production broth). When describing a two phase system the P is usually discussed in terms of log P. A compound with a log P of 1 would partition 10:1 to the organic phase, while a compound of log P of 0.1 would partition 1:10 to the organic phase. One or ordinary skill in the art will appreciate that by choosing a production broth and the organic phase such that the fatty acid derivative being produced has a high log P value, the fatty acid derivative will separate into the organic phase, even at very low concentrations in the production vessel.

The fatty acid derivatives produced by the methods described herein will be relatively immiscible in the production broth, as well as in the cytoplasm. Therefore, the fatty acid derivative will collect in an organic phase either intracellularly or extracellularly. The collection of the products in an organic phase will lessen the impact of the fatty acid derivative on cellular function and will allow the production host to produce more product. Stated another way, the concentration of the fatty acid derivative will not have as significant of an impact on the host cell.

The fatty esters produced as described herein allow for the production of homogeneous compounds wherein at least 60%, 70%, 80%, 90%, 95%, 98%, 99%, or 100% by volume of the fatty esters produced will have carbon chain lengths that vary by less than 4 carbons, or less than 2 carbons. These compounds can also be produced so that they have a relatively uniform degree of saturation, for example at least 60%, 70%, 80%, 90%, 95%, 98%, 99%, or 100% by volume of the fatty esters will be mono-, di-, or tri-unsaturated. These compounds can be used directly as fuels, personal care additives, or nutritional supplements. These compounds can also be used as feedstock for subsequent reactions, for example transesterification, hydrogenation, catalytic cracking via either hydrogenation, pyrolisis, or both or epoxidations reactions, to make other products. The fatty esters can also be concentrated such that the composition of which they are part will comprise at least about 80% fatty esters, for example, the percent fatty ester can be about 80-85, 85-90, 90-95, 95-99% or more.

In some embodiments, in order to be used as a biofuel, the fatty ester composition can be further processed. In some embodiments, the fatty ester composition can be isolated from the broth and the cells. In addition, the fatty ester composition can be purified to remove excess water. In some embodiments, fine solids can be removed that might affect injection nozzles or prefilters in engines. In some embodiments, the fatty ester composition can also be processed to remove species that have poor volatility and would lead to deposit formation. In some embodiments, traces of sulfur compounds that may be present are removed. In some embodiments, the above can be achieved via one or more of the following: washing, adsorption, distillation, filtration, centrifugation, settling, and coalescence.

In some embodiments, during processing, impurities in the alcohol can enter the fermentation off gas. Off gas treatment steps can be used as appropriate depending on the impurity.

### Fatty Acid Derivatives

The centane number (CN), viscosity, melting point, and heat of combustion for various fatty esters have been characterized in for example, Knothe, Fuel Processing Technology 86:1059-1070, 2005, which is herein incorporated by reference. Using the teachings provided herein a production host can be engineered to produce any one of the fatty esters described in the Knothe, Fuel Processing Technology 86:1059-1070, 2005.

Alcohols (short chain, long chain, branched or unsaturated) can be produced by the production hosts described herein. Such alcohols can be used as fuels directly. Alternatively, they can be used to create a fatty ester (i.e. the A side of a fatty ester) as described above. Such fatty esters alone, or in combination with the other fatty acid derivatives described herein, are useful a fuels.

### Reduced Impurities

In some embodiments, the fatty acid derivatives described herein can be useful for making biofuels. In some embodiments, these fatty acid derivatives are made directly from fatty acids. Accordingly, in some embodiments, fuels comprising the disclosed fatty acid derivatives can contain less of some types of impurities than are normally associated with biofuels derived from triglycerides, such as fuels derived from vegetable oils and fats.

The crude fatty acid derivative biofuels described herein (prior to mixing the fatty acid derivative with other fuels, such as traditional fuels) will contain less transesterification catalyst than petrochemical diesel or biodiesel. For example, the fatty acid derivative can contain less than about 2%, 1.5%, 1%, 0.5%, 0.3%, 0.1%, 0.05%, or 0% by volume of a transesterification catalyst or an impurity resulting from a transesterification catalyst. Transesterification catalysts include, for example, hydroxide catalysts, such as NaOH, KOH, LiOH, and acidic catalysts, such as mineral acid catalysts and Lewis acid catalysts. Catalysts and impurities resulting from transesterification catalysts include, without limitation, tin, lead, mercury, cadmium, zinc, titanium, zirconium, hafnium, boron, aluminum, phosphorus, arsenic, antimony, bismuth, calcium, magnesium, strontium, uranium, potassium, sodium, lithium, and combinations thereof.

Similarly, the crude fatty acid derivative biofuels described herein (prior to mixing the fatty acid derivative with other fuels such as petrochemical diesel or biodiesel) will contain less glycerol (or glycerin) than bio-fuels made from triglycerides. For example, the fatty acid derivative can contain less than about 2%, 1.5%, 1%, 0.5%, 0.3%, 0.1%, 0.05%, or 0% glycerol.

The crude biofuel derived from fatty acid derivatives will also contain less free alcohol (i.e., alcohol that is used to create the ester) than biodiesel made from triglycerides. This is, in part, due to the efficiency of utilization of the alcohol by the production host. For example, the fatty acid derivative will contain less than about 2%, 1.5%, 1%, 0.5%, 0.3%, 0.1%, 0.05%, or 0% free alcohol.

Biofuel derived from the disclosed fatty acid derivatives can be additionally characterized by its low concentration of sulfur compared to petroleum derived diesel. For example, biofuel derived from fatty acid derivatives can have less than about 2%, 1.5%, 1%, 0.5%, 0.3%, 0.1%, 0.05%, or 0% sulfur.

In some embodiments, while the biofuel, fatty ester, or fatty ester derivative has less of one or more of the above impurities, it has more of another impurity. For example, the biofuel, fatty ester, or fatty acid derivative can have additional impurities from those of unrefined or impure alcohols (e.g., ethanol) as noted above. Thus, in some embodiments, the biofuel, fatty ester, or fatty acid derivative can have more of some types of impurities (e.g., those present in an impure alcohol) and less of the impurities discussed within this section.

### Fuel and Fatty Ester Compositions

The fatty acid derivatives described herein can be used as a fuel. One of ordinary skill in the art will appreciate that depending upon the intended purpose of the fuel, different fatty acid derivatives can be produced and used. For example, for automobile fuel that is intended to be used in cold climates, a branched fatty acid derivative can be desirable. Using the teachings provided herein, branched hydrocarbons, fatty esters, and alcohols can be made. Using the methods described herein, fuels comprising relatively homogeneous fatty acid derivatives that have desired fuel qualities can be produced. Such fuels can be characterized by carbon fingerprinting or their lack of impurities when compared to petroleum derived fuels or biodiesel derived from triglycerides. Moreover, the fatty acid derivative based fuels can be combined with other fuels or fuel additives to produce fuels having desired properties.

In some embodiments, the fatty ester composition comprises a variety of fatty esters that can vary in A₁ and B₁ length, saturation level, and ratios between the different species. Thus, in some embodiments, B₁ can be a 6, 7, 8, 9, 10, 11, 12, 13, 14, 15, 16, 17, 18, 19, 20, 21, 22, 23, 24, 25, 26, 27, 28, 29, or 30 carbon chain which can have 1, 2, 3, 4, 5, 6, 7, 8, 9, 10, 11, 12, 13, 14, 15, 16, 17, 18, 19, 20, 21, 22, 23, or 24 double bonds. 1-24 of those double bonds can be located following carbon 1, 2, 3, 4, 5, 6 7, 8, 9, 10, 11, 12, 13, 14, 15, 16, 17, 18, 19, 20, 21, 22, 23, 24, 25, 26, 27, 28, or 29. A₁ can be a 1, 2, 3, 4, 5, or 6 carbon chain having 1, 2, 3, 4, or 5 double bonds. 1-5 of those double bonds can be located following carbon 1, 2, 3, 4, or 5. One or more of these A₁COOB₁ species (each different species denoted as A₂COOB₂, A₃COOB₃, etc.) can make up some fraction of the fatty ester composition. Thus, in some embodiments, one or more of the above species makes up at least about 1%, 5%, 10%, 15%, 20%, 25%, 30%, 35%, 40%, 50%, 60%, 70%, 80%, 90%, or 95% by volume of the fatty ester composition. In some embodiments, the fatty ester composition is at least about 50 to about 95 wt % C_{16:1} ethyl ester, at least about 50 to about 95 wt % C_{18:1} ethyl ester, at least about 50 to about 95 wt % C_{16:0} ethyl ester, and/or at least about 50 to about 95 wt % C_{18:0} ethyl ester. In some embodiments, the fatty ester composition is at least about 50 to about 100 wt % C_{16:1} ethyl ester, at least about 50 to about 100 wt % C_{18:1} ethyl ester, at least about 50 to about 100 wt % C_{16:0} ethyl ester, and/or at least about 50 to about 100 wt % C_{18:0} ethyl ester. In some embodiments, the fatty ester composition comprises about 20%, 30%, 40%, 50%, 60%, 70%, 80%, 85%, 90%, or 95% fatty ester that has a B₁ carbon chain that is 8:0, 10:0, 12:0, 14:0, 14:1, 16:0, 16:1, 18:0, 18:1, 18:2, 18:3, 20:0, 20:1, 20:2, 20:3, 22:0, 22:1, or 22:3.

### Additives

In some embodiments, fuel additives are used to enhance the performance of a fuel or engine. For example, fuel additives can be used to alter the freezing/gelling point, cloud point, lubricity, viscosity, oxidative stability, ignition quality, octane level, and flash point. In the United States, all fuel additives must be registered with the Environmental Protection Agency (EPA). Companies that sell fuel additives and the name of the fuel additive are publicly available on the EPA's website or also by contacting the EPA. One of ordinary skill in the art will appreciate that the fatty acid derivatives described herein can be mixed with one or more such additives to impart a desired quality.

One of ordinary skill in the art will also appreciate that the fatty acid derivatives described herein can be mixed with other fuels, such as biodiesel derived from triglycerides, various alcohols, such as ethanol and butanol, and petroleum derived products, such as gasoline. In some examples, a fatty acid derivative, such as C16:1 ethyl ester or C18:1 ethyl ester, is produced which has a low gel point. This low gel point fatty acid derivative is mixed with biodiesel made from triglycerides to lessen the overall gelling point of the fuel. Similarly, a fatty acid derivative, such as C16:1 ethyl ester or C18:1 ethyl ester, can be mixed with petroleum derived diesel to provide a mixture that is at least and often greater than 5% biodiesel. In some examples, the mixture includes at least about 20% or greater of the fatty acid derivative.

For example, a biofuel composition can be made that includes at least about 20%, 30%, 40%, 50%, 60%, 70%, 80%, 85%, 90% or 95% by volume of a fatty acid derivative and/or fatty ester that includes a carbon chain that is 8:0, 10:0, 12:0, 14:0, 14:1, 16:0, 16:1, 18:0, 18:1, 18:2, 18:3, 20:0, 20:1, 20:2, 20:3, 22:0, 22:1 or 22:3. Such biofuel compositions can additionally include at least one additive selected from a cloud point lowering additive that can lower the cloud point to less than about 5° C., or 0° C., a surfactant, or a microemulsion, at least about 5%, 10%, 15%, 20%, 30%, 40%, 50%, 60%, 70% or 80%, 85%, 90%, or 95% diesel fuel from triglycerides, petroleum derived gasoline or diesel fuel from petroleum.

As will be appreciated by one of skill in the art, any of the above fatty esters and fatty ester compositions can be converted into a biofuel, or more specifically biodiesel, if desired. Thus, the corresponding biofuels and biodiesels are also provided herein.

### EXAMPLES

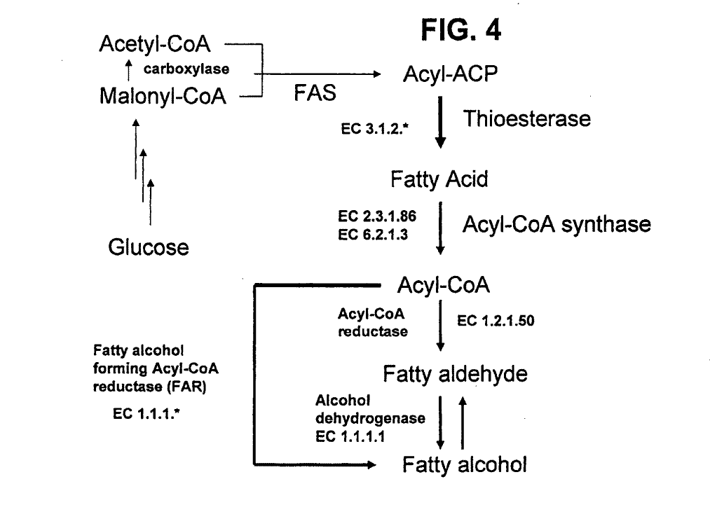

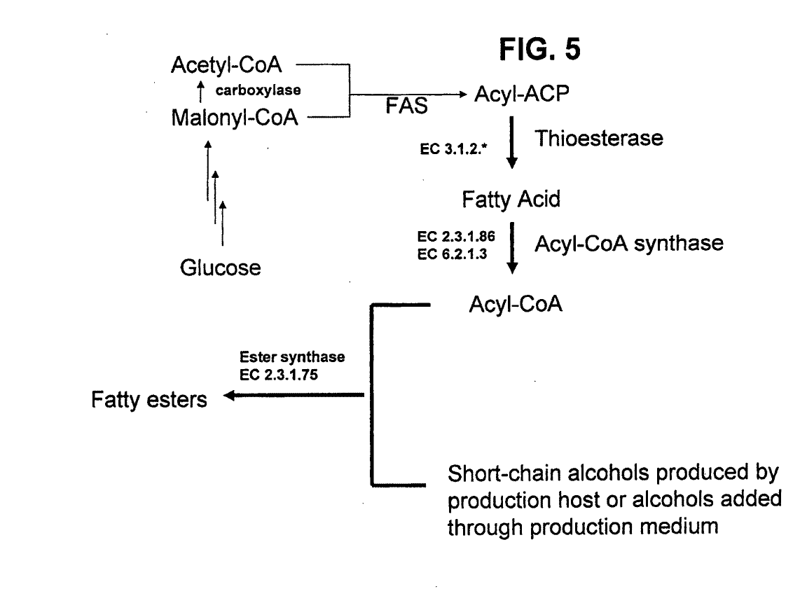

The examples provided herein illustrate the engineering of production hosts to produce specific fatty esters. Exemplary biosynthetic pathways involved in the production of fatty esters are illustrated in the figures. For example, FIG. 2 is a diagram of the FAS pathway showing the enzymes directly involved in the synthesis of acyl-ACP. To increase the production of fatty esters, one or more of the enzymes in FIG. 2 can be over expressed or mutated to reduce feedback inhibition to increase the amount of acyl-ACP produced. Additionally, enzymes that metabolize the intermediates to make non-fatty acid based products (side reactions) can be functionally deleted or attenuated to increase the flux of carbon through the fatty acid biosynthetic pathway. In the examples below, many production hosts are described that have been modified to increase fatty acid production. FIG. 3, FIG. 4, and FIG. 5 show biosynthetic pathways that can be engineered to make fatty esters. As illustrated in FIG. 3, the conversion of each substrate (e.g., acetyl-CoA, malonyl-CoA, acyl-ACP, fatty acid, and acyl-CoA) to each product (e.g., acetyl-CoA, malonyl-CoA, acyl-ACP, fatty acid, and acyl-CoA) can be accomplished using several different polypeptides that are members of the enzyme classes indicated.

The examples below describe microorganisms that have been engineered or can be engineered to produce specific fatty esters.

### Example 1

### Production Host Construction

The present example outlines various production hosts and methods of making them. An exemplary production host is E. coli. A preferred production host is E. coli with the fadE gene attenuated or deleted. An E. coli lacking the fadE gene was produced by modifying E. coli C41(DE3) from OverExpress (Saint Beauzire, France) to knock-out the fadE gene (acyl-CoA dehydrogenase).

Briefly, the fadE knock-out strain of E. coli was made using primers YafV_NotI and Ivry_Ol to amplify about 830 by upstream of fadE and primers Lpcaf_ol and LpcaR_Bam to amplify about 960 by downstream of fadE. Overlap PCR was used to create a construct for in-frame deletion of the complete fadE gene. The fadE deletion construct was cloned into the temperature-sensitive plasmid pKOV3, which contained a sacB gene for counterselection, and a chromosomal deletion of fadE was made according to the method of Link et al., J. Bact. 179:6228-6237, 1997. The resulting strain was not capable of degrading fatty acids and fatty acyl-CoAs. This knock-out strain is herein designated as E. coli C41 (DE3, ΔfadE).

An additional production host was made that included a plasmid carrying the four genes that are responsible for acetyl-CoA carboxylase activity in E. coli (accA, accB, accC, and accD, Accessions: NP_{—}414727, NP_{—}417721, NP_{—}417722, NP_{—}416819, respectively, EC 6.4.1.2). The accABCD genes were cloned in two steps as bicistronic operons into the NcoI/HindIII and NdeI/AvrII sites of pACYCDuet-1 (Novagen, Madison, Wis.), and the resulting plasmid was termed pACYCDuet-1-accABCD. This also included the ΔfadE modification noted above.

Similarly, a production host can be engineered to express accABCD (encoding acetyl-CoA carboxylase) from Pisum savitum. However, when the production host is also producing butanol, it is less desirable to express accABCD from Pisum savitum.

Additional production host modifications that can be made include the following adjustments: overexpression of aceEF (encoding the E1p dehydrogase component and the E2p dihydrolipoamide acyltransferase component of the pyruvate and 2-oxoglutarate dehydrogenase complexes) or fabH/fabD/fabG/acpP/fabF (encoding FAS) from E. coli, Nitrosomonas europaea (ATCC 19718), Bacillus subtilis, Saccharomyces cerevisiae, Streptomyces spp, Ralstonia, Rhodococcus, Corynebacteria, Brevibacteria, Mycobacteria, or oleaginous yeast.

In some exemplary production hosts, genes can be knocked out or attenuated using the method of Link, et al., J. Bacteria 179:6228-6237, 1997. For example, genes that can be knocked out or attenuated include gpsA (encoding biosynthetic sn-glycerol 3-phosphate dehydrogenase, accession NP_{—}418065, EC: 1.1.1.94); ldhA (encoding lactate dehydrogenase, accession NP_{—}415898, EC: 1.1.1.28); pflb (encoding formate acetyltransferase 1, accessions: P09373, EC: 2.3.1.54); adhE (encoding alcohol dehydrogenase, accessions: CAA47743, EC: 1.1.1.1, 1.2.1.10); pta (encoding phosphotransacetylase, accessions: NP_{—}416800, EC: 2.3.1.8); poxB (encoding pyruvate oxidase, accessions: NP_{—}415392, EC: 1.2.2.2); ackA (encoding acetate kinase, accessions: NP_{—}416799, EC: 2.7.2.1) and combinations thereof.

In another embodiment, the plsB[D311E] mutation can be introduced into E. coli C41 (DE3, ΔfadE) to attenuate plsB using the method described above for the fadE deletion. This mutation decreases the amount of carbon diverted to phospholipid production (see FIG. 2). An allele encoding plsB[D311E] can be made by replacing the GAC codon for aspartate 311 with a GAA codon for glutamate. The altered allele can be made by gene synthesis, and the chromosomal plsB wildtype allele can be exchanged for the mutant plsB[D311E] allele using the method of Link et al. (see above).

For the commercial production of fatty acid derivatives via fermentation, the production host's internal regulatory pathways can be optimized to produce more of the desired products. In many instances, this regulation can be optimized by overexpressing certain enzymes. Some examples are shown in Table 7.

TABLE 7
Additional genes that can be optimized for fatty acid derivative production

| | | Example of E. coli |
|---|---|---|
| | | gene(s) |
| | EC | (or other |
| Enzymatic Activity | Number | microorganism) |
| Pantetheine-phosphate adenylyltransferase | 2.7.7.3 | coaD |
| Dephospho-CoA kinase | 2.7.1.24 | coaE |
| Biotin-[acetyl-CoA-carboxylase] ligase | 6.3.4.15 | birA |
| Carbonic anhydrase | 4.2.1.1 | cynT, can(yadF) |
| Apo-[acyl carrier protein] | None | acpP |
| Holo-[acyl-carrier-protein] synthase | 2.7.8.7 | acpS, acpT |
| Pyruvate dehydogenase complex | 1.2.4.1 | aceF |
| | 2.3.1.12 | aceE |
| | 1.8.1.4 | Lpd |
| NAD Kinase | 2.7.1.23 | nadK (yfjB) |
| Pyruvate-ferredoxin oxidoreductase | 1.2.7.1 | porA (Desulfovobrio |
| | | vulgaris DP4) |

### Example 2

### Additional Production Hosts

The present example outlines additional modifications that were made to various production hosts.

The following plasmids were constructed for the expression of various genes that are used in the synthesis of fatty acid derivatives. The constructs were made using standard molecular biology methods. The cloned genes were put under the control of IPTG-inducible promoters (e.g., T7, tac, or lac promoters).

The 'tesA gene (thioesterase A gene accession NP_{—}415027 without a leader sequence (See, e.g., Cho and Cronan, J. Biol. Chem., 270:4216-9, 1995, EC: 3.1.1.5, 3.1.2.-)) of E. coli was cloned into NdeI/AvrII digested pETDuet-1 (pETDuet-1 described herein is available from Novagen, Madison, Wis.). Genes encoding for FatB-type plant thioesterases (TEs) from Umbellularia californica, Cuphea hookeriana, and Cinnamonum camphorum (Accessions: UcFatB1=AAA34215, ChFatB2=AAC49269, ChFatB3=AAC72881, CcFatB=AAC49151, respectively) were individually cloned into three different vectors: (i) NdeI/AvrII digested pETDuet-1; (ii) XhoI/HindIII digested pBluescript KS+ (Stratagene, La Jolla, Calif., to create N-terminal lacZ::TE fusion proteins); and (iii) XbaI/HindIII digested pMAL-c2X (New England Lab, Ipswich, Mass.) (to create n-terminal malE::TE fusions).

The fadD gene (encoding acyl-CoA synthase) from E. coli was cloned into a NcoI/HindIII digested pCDFDuet-1 derivative that also contained the acr1 gene (acyl-CoA reductase) from Acinetobacter baylyi ADP1 within its NdeI/AvrII sites.

Table 8 provides a summary of the plasmids generated to make several exemplary production hosts.

The chosen expression plasmids contained compatible replicons and antibiotic resistance markers to produce a four-plasmid expression system.

TABLE 8
Summary of plasmids used in production hosts

| | Source Organism | Accession No., |
|---|---|---|
| Plasmid | Gene Product | EC number |
| pETDuet-1-tesA | E. coli | Accessions: |
| | tesA | NP_415027, |
| | | EC: 3.1.1.5, |
| | | 3.1.2.— |
| pETDuet-1-TEuc | Umbellularia californica | Q41635 |
| pBluescript-TEuc | UcFatB1 | |
| pMAL-c2X-TEuc | | AAA34215 |
| pETDuet-1-TEchfatB2 | Cuphea hookeriana | |
| pBluescript-TEchfatB2 | ChFatB2 | AAC49269 |
| pMAL-c2X-TEchfatB2 | | |
| pETDuet-1-TEchfatB3 | Cuphea hookeriana | AAC72881 |
| pBluescript-TEchfatB3 | ChFatB3 | |
| pMAL-c2X-TEchfatB3 | | |
| pETDuet-1-TEcc | Cinnamonum camphorum | |
| pBluescript-TEcc | CcFabB | AAC49151 |
| TEci | | |
| pETDuet-1-atFatA3 | Arabidopsis thaliana | NP_189147 |
| pETDuet-1-haFatA1 | Helianthus annuus | AAL769361 |
| pCDFDuet-1-fadD | E. coli | fadD: Accessions |
| | | NP_416319, |
| | | EC 6.2.1.3 |
| pCDFDuet-1-fadD-acrl | fadD: E. coli | fadD: Accessions |
| | acrl: Acinetobacter | NP_416319, |
| | baylyi ADP1 | EC 6.2.1.3 |
| | | acrl: Accessions |
| | | AF529086.1 |
| pCDFDuet-1-fadD-atfA | fadD: E. coli | fadD: Accessions |
| | atfA: Acinetobacter | NP_416319, |
| | baylyi ADP1 | EC 6.2.1.3 |
| | | atfA: Accessions |
| | | AF529086.1 |
| pRSET-B-saat | | AF193789 |
| pCOLADuet-1-atfA | atfA: Acinetobacter | atfA: Accessions |
| | baylyi ADP1 | AF529086.1 |

One of ordinary skill in the art will appreciate that different plasmids and genomic modifications can be used to achieve similar strains to those described herein.

In some embodiments, E. coli C41 (DE3, ΔfadE) can be co-transformed with: (i) any of the thioesterase (e.g., TesA) expressing plasmids; (ii) an acyl-CoA synthase (e.g., FadD) expressing plasmid, which also expresses an acyl-CoA reductase (e.g., Acr1); and (iii) an ester synthase expression plasmid.

As will be clear to one of skill in the art, when E. coli C41 (DE3, ΔfadE) is induced with IPTG, the resulting strain will produce increased concentrations of fatty esters from carbon sources, such as glucose.

### Example 3

### Medium Chain Fatty Esters

Alcohol acetyl transferases (AATs, EC 2.3.1.84), which are responsible for acyl acetate production in various plants, can be used to produce medium chain length fatty esters (e.g., octyl octanoate, decyl octanoate, decyl decanoate, etc.). Fatty esters, synthesized from medium chain alcohols (e.g., C₆ to C₈), medium chain acyl-CoA, or medium chain fatty acids (e.g., C₆ to C₈), have a relatively low melting point. For hexyl hexanoate has a melting point of −55° C. and octyl octanoate has a melting point of −18 to −17° C. The low melting points of these compounds make them good candidates for use as biofuels.

In this example, an SAAT gene encoding an alcohol acetyltransferase was co-expressed in production host E. coli C41(DE3, ΔfadE) with fadD from E. coli and acr1 (acyl-CoA reductase from A. baylyi ADP1). Octanoic acid was provided in the fermentation broth. This resulted in the production of octyl octanoate. Similarly, when the ester synthase gene from A. baylyi ADP1 was expressed in the production host instead of the SAAT gene, octyl octanoate was produced.

A recombinant SAAT gene was synthesized by DNA 2.0 (Menlo Park, Calif. 94025). The synthesized DNA sequence was based on the published gene sequence (accession number AF193789), but modified to eliminate the NcoI site. The synthesized SAAT gene (as a BamHI-HindIII fragment) was cloned in pRSET B (Invitrogen, Carlsbad, Calif.), linearized with BamHI and HindIII. The resulting plasmid, pRSET-B-saat, was cotransformed into an E. coli production host with pCDFDuet-1-fadD-acr1, which carries a fadD gene from E. coli and acr1 gene from A. baylyi ADP1. The transformants were grown in 3 mL of M9 medium with 2% glucose. After IPTG induction and the addition of 0.02% octanoic acid, the culture was continued at 25° C. for 40 hours. 3 mL of ethyl acetate was then added to the whole culture and mixed several times with a mixer. The ethyl acetate phase was analyzed by GC/MS.

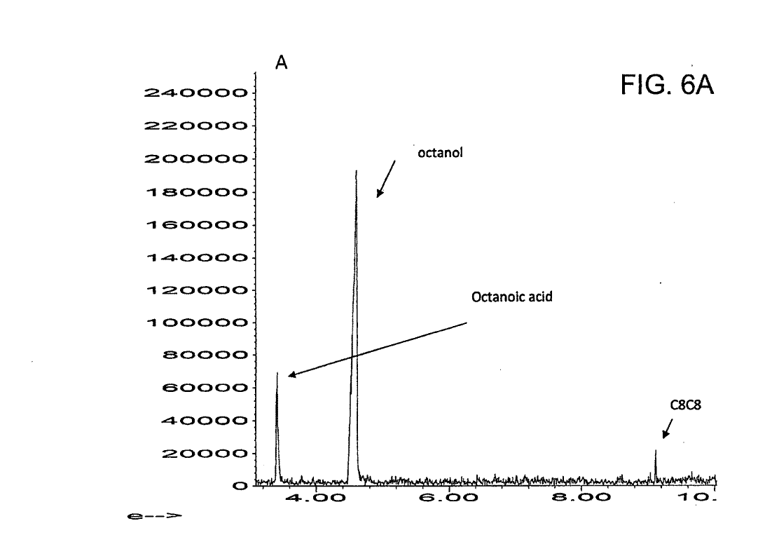

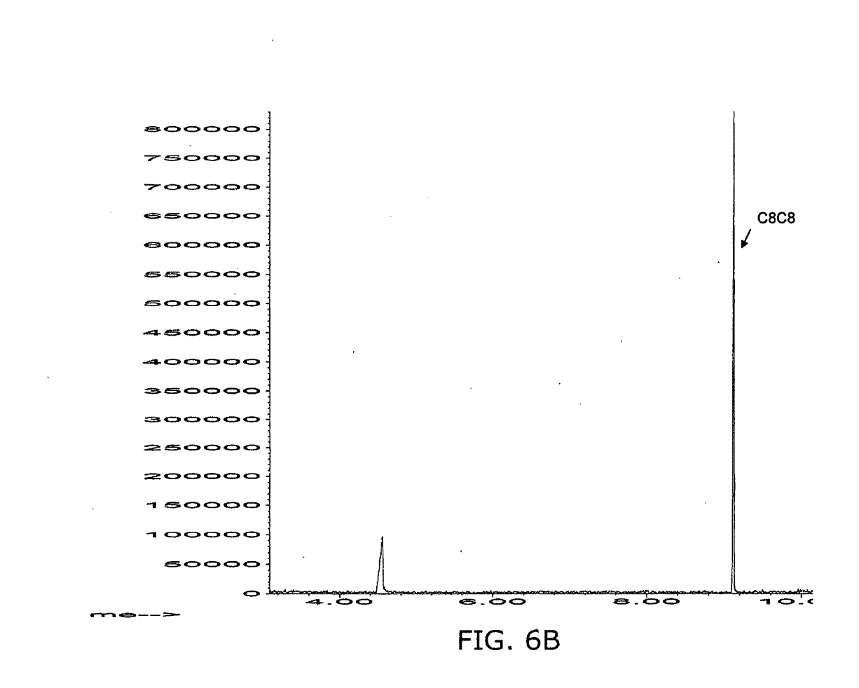

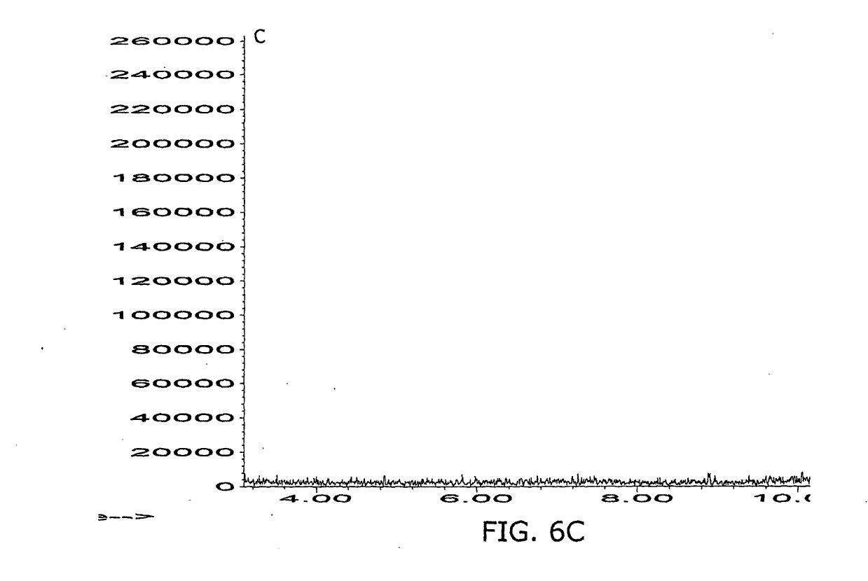

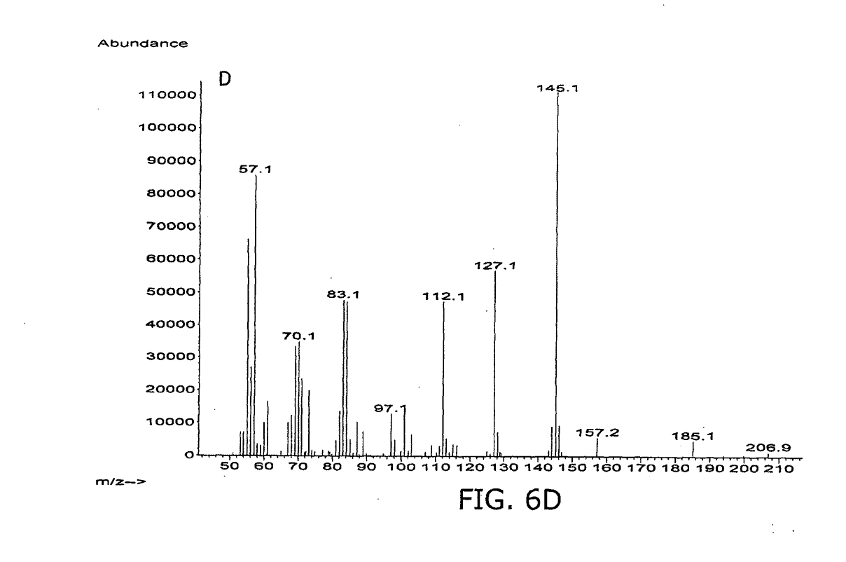

Surprisingly, no acyl acetate was observed in the ethyl acetate extract. However, octyl octanoate was observed. However, the control strain without the SAAT gene (E. coli C41(DE3, ΔfadE)/pRSET B/pCDFDuet-1-fadD-acr1) did not produce octyl octanoate (FIGS. 6A and 6C). Furthermore, the strain (E. coli C41(DE3, ΔfadE)/pCOLADuet-1-atfA/pCDFDuet-1-fadD-acr1) in which the ester synthase gene from A. baylyi ADP1 was carried by pCOLADuet-1-atfA produced octyl octanoate (FIGS. 6B and 6D).

The finding that SAAT activity produces octyl octanoate makes it possible to produce medium chain fatty esters, such as octyl octanoate and octyl decanoate, which have low melting points and are good candidates for use as biofuels to replace triglyceride based biodiesel.

### Example 4

### Production and Release of Fatty Ethyl Ester from Production Host

The present example outlines how to produce a fatty ester by using an E. coli C41(DE3, ΔfadE) production host.

The E. coli C41 (DE3, ΔfadE) production host was transformed with plasmids carrying an ester synthase gene from A. baylyi (plasmid pCOLADuet-1-atfA), a thioesterase gene from Cuphea hookeriana (plasmid pMAL-c2X-TEchfatB3), and a fadD gene from E. coli (plasmid pCDFDuet-1-fadD).

This recombinant strain was grown at 25° C. in 3 mL of M9 medium with 50 mg/L kanamycin, 100 mg/L carbenicillin, and 100 mg/L of spectinomycin. After IPTG induction, the media was adjusted to a final concentration of 1% ethanol and 2% glucose.

The culture was allowed to grow for 40 hours after IPTG induction. The cells were separated from the spent medium by centrifugation at 3500×g for 10 minutes. The cell pellet was re-suspended with 3 mL of M9 medium. The cell suspension and the spent medium were then extracted with 1 volume of ethyl acetate. The resulting ethyl acetate phases from the cells suspension and the supernatant were subjected to GC-MS analysis.

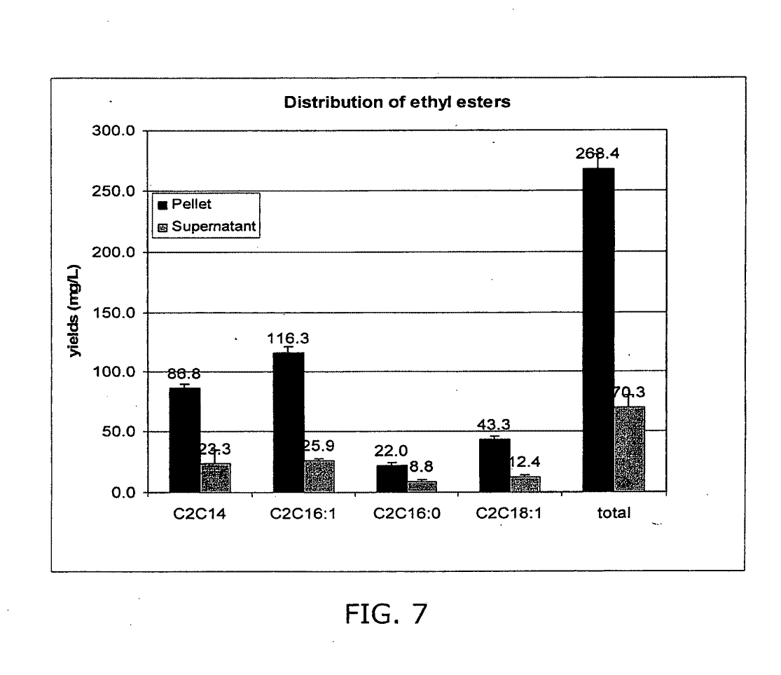

The C₁₆ ethyl ester was the most prominent ester species (as expected for this thioesterase, see Table 1), and 20% of the fatty ester produced was released from the cell (see FIG. 7). A control E. coli strain E. coli C41(DE3, ΔfadE) containing pCOLADuet-1 (empty vector for the ester synthase gene), pMAL-c2X-TEchfatB3 (containing fatB3 from Cuphea hookeriana), and pCDFDuet-1-fadD (fadD gene from E. coli) failed to produce detectable amounts of fatty esters. The fatty esters were quantified using commercial palmitic acid ethyl ester as the reference.

Fatty esters were also made using the methods described herein except that methanol or isopropanol was added to the production broth. The predicted fatty esters were produced.

### Example 5

### Alternative Production Hosts and Uses Thereof

The present example examines the influence of various thioesterases on the composition of fatty ethyl esters produced in recombinant E. coli strains.

The thioesterases FatB3 (C. hookeriana), TesA (E. coli), and FatB3 (U. california) were expressed simultaneously with ester synthase (A. baylyi). A plasmid, pCDFDuet-1-fadD-atfA, was constructed by replacing the NotI-AvrII fragment (carrying the acr1 gene) with the NotI-AvrII fragment from pCOLADuet-1-atfA so that fadD and the atfA ester synthase were in one plasmid and both coding sequences were under the control of separate T7 promoters. The construction of pCDFDuet-1-fadD-atfA made it possible to use a two plasmid system. pCDFDuet-1-fadD-atfA was then co-transformed into E. coli C41(DE3, ΔfadE) with one of the various plasmids carrying the different thioesterase genes stated above.

The total fatty ethyl esters (in both the supernatant and intracellular fatty ethyl fluid) produced by these transformants were evaluated using the technique described herein. The yields and the composition of fatty ethyl esters are summarized in Table 9. With regard to Table 9, the following plasmids were used: 'TesA, pETDuet-1-'tesA; chFatB3, pMAL-c2X-TEchfatB3; ucFatB, pMAL-c2X-TEuc; pMAL, pMAL-c2X, the empty vector for thioesterase genes.

TABLE 9

| Thioesterases | C₂C₁₀ | C₂C_{12:1} | C₂C₁₂ | C₂C_{14:1} | C₂C₁₄ | C₂C_{16:1} | C₂C₁₆ | C₂C_{18:1} | Total |
|---|---|---|---|---|---|---|---|---|---|
| ‘TesA | 0.0 | 0.0 | 6.5 | 0.0 | 17.5 | 6.9 | 21.6 | 18.1 | 70.5 |
| ChFatB3 | 0.0 | 0.0 | 0.0 | 0.0 | 10.8 | 12.5 | 11.7 | 13.8 | 48.8 |
| ucFatB | 6.4 | 8.5 | 25.3 | 14.7 | 0.0 | 4.5 | 3.7 | 6.7 | 69.8 |
| pMAL | 0.0 | 0.0 | 0.0 | 0.0 | 5.6 | 0.0 | 12.8 | 7.6 | 26.0 |

### Example 6

### Additional Exemplary Production Hosts

The present example provides additional alternative productions host and or specific genes that can be employed in various embodiments described herein.

Various production hosts can be used, such as: a mammalian cell, plant cell, insect cell, yeast cell, fungus cell, filamentous fungi cell, bacterial cell, a Grain-positive bacteria, a Gram-negative bacteria, the genus Escherichia, the genus Bacillus, the genus Lactobacillus, the genus Rhodococcus, the genus Pseudomonas, the genus Aspergillus, the genus Trichoderma, the genus Neurospora, the genus Fusarium, the genus Humicola, the genus Rhizomucor, the genus Kluyveromyces, the genus Pichia, the genus Mucor, the genus Myceliophtora, the genus Penicillium, the genus Phanerochaete, the genus Pleurotus, the genus Trametes, the genus Chrysosporium, the genus Saccharomyces, the genus Stenotrophamonas, the genus Schizosaccharomyces, the genus Yarrowia, the genus Streptomyces, a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, a Mucor michei cell, a Streptomyces lividans cell, a Streptomyces murinus cell, an Actinomycetes cell, a CHO cell, a COS cell, a VERO cell, a BHK cell, a HeLa cell, a Cv1 cell, an MDCK cell, a 293 cell, a 3T3 cell, a PC12 cell, an E. coli cell, a strain B E. coli cell, a strain C E. coli cell, a strain K E. coli cell, and a strain W E. coli cell. The production hosts can be used as described in the examples above.

There are a variety of genes with the same function from other organisms that can be used to achieve the specific desired results in various production hosts. For example, one need not use tesA, but could use any thioesterase, such as: ccFatB (Cinnamomum camphora), umFatB (Umbellularia californica), chFatB2 (Cuphea hookeriana), chFatB3 (Cuphea hookeriana), chFatA (Cuphea hookeriana), atFatA1 (Arabidopsis thaliana), or atFatB1[M141T] (Arabidopsis thaliana).

Exemplary acyl-CoA reductases which can be used are: bFAR (Bombyx mori), acr1 (Acinetobacter baylyi ADP1), jjFAR (Simmondsia chinensis), TTA1 (Triticum aestivum), mFAR1 (Mus musculus), mFAR2 (Mus musculus), acr M1 (Acinetobacter sp M1), or hFAR (Homo sapiens)

Exemplary ester synthases which can be used are: WST9 (Fundibacter jadensis DSM 12178), WSHN (Acinetobacter sp. HO1-N), WSadp1 (Acinetobacter baylyl ADP1), mWS (Mus musculus), hWS (Homo sapiens), SAAT (Fragaria ananassa), mpAAT (Malus domestica), or jjWS (Simmondsia chinensis).

An exemplary decarbonylase which can be used is cer1 (Arabidopsis thaliana).

An exemplary transport protein which can be used is cer5 (Arabidopsis thaliana).

### Example 7

### Exemplary Production Process

The present example describes one example for part of a production process.

Production hosts are engineered to express umuC and umuD from E. coli in pBAD24 under the prpBCDE promoter system through de novo synthesis of this gene with the appropriate end-product production genes. For small scale hydrocarbon product production, E. coli BL21(DE3) cells harbouring pBAD24 (with ampicillin resistance and the end-product synthesis pathway) as well as pUMVC1 (with kanamycin resistance and the acetyl-CoA/malonyl-CoA over-expression system) are incubated overnight at 37° C. and shaken at >200 rpm in 2 L flasks in 500 ml LB medium supplemented with 75 micrograms/mL ampicillin and 50 micrograms/ml kanamycin until cultures reach an OD₆₀₀ of >0.8. Upon achieving an OD₆₀₀ of >0.8 cells are supplemented with 25 mM sodium proprionate (pH 8.0) to activate the engineered gene systems for production as well as to stop cellular proliferation (through activation of umuC and umuD proteins). Induction is performed for 6 hours at 30° C. After incubation, production media is examined for product using GC-MS (as described herein).

For large scale product production, the engineered microorganisms can be grown in 10 L, 100 L, 10×10⁵ L, 2 million, 3 million 3.5 million, 3.8 million, or larger batches and manipulated to express desired products based on the specific genes encoded in the plasmids, as appropriate.

E. coli BL21(DE3) cells harbouring pBAD24 (with ampicillin resistance and the end-product synthesis pathway) as well as pUMVC1 (with kanamycin resistance and the acetyl-CoA/malonyl-CoA over-expression system) are incubated from a 500 mL seed culture for 10 L fermentations (5 L for 100 L fermentations) in LB media (glycerol free) at 37° C. shaken at >200 rpm until cultures reach an OD₆₀₀ of >0.8 (typically about 16 hours) with 50 micrograms/mL kanamycin and 75 micrograms/mL ampicillin. The production media is supplemented to maintain a 25 mM sodium proprionate (pH 8.0) to activate the engineered gene systems for production as well as to stop cellular proliferation (through activation of umuC and umuD proteins). Media is continuously supplemented with glucose to maintain a concentration of 10 g/100 mL. After the first hour of induction, aliquots of no more than 10% of the total culture volume are removed each hour and are allowed to sit unagitated so as to allow the hydrocarbon product to rise to the surface and undergo a spontaneous phase separation. The hydrocarbon component is then collected, and the aqueous phase is returned to the reaction chamber. The reaction chamber is operated continuously. When the OD₆₀₀ drops below 0.6, the cells are replaced with a new batch grown from a seed culture.

While the above example outlines one embodiment for how the production process can occur, as will be appreciated by one of skill in the art, additional processing or refinement can occur to the product. In some embodiments, such as in fatty ester production, subsequent to isolation, the fatty esters can be washed briefly in 1 M HCl to split the ester bond and to return the pH to 7 with extensive washing with distilled water. In some embodiments, the product can be purified to remove excess water. In some embodiments, fine solids can be removed that might affect injection nozzles or prefilters in engines. In some embodiments, the fatty ester can also be processed to remove species that have poor volatility and would lead to deposit formation. Traces of sulfur compounds that may be present can be removed. It will be appreciated that steps for removing substances from the product can include one or more of washing, adsorption, distillation, filtration, centrifugation, settling, or coalescence.

### Example 8

### Product Characterization

The present example outlines an embodiment for characterizing a product of a production host.

To characterize and quantify the fatty esters and other compounds, gas chromatography (GC) coupled with electron impact mass spectra (MS) detection was used. Fatty esters did not required derivatization. Fatty esters were dissolved in an appropriate volatile solvent, such as ethyl acetate.

The samples were analyzed on a 30 m DP-5 capillary column using the following method. After a 1 μL splitless injection onto the GC/MS column (inlet temperature held at 300° C.), the oven was held at 100° C. for 3 minutes. The temperature was ramped up to 320° C. at a rate of 20° C./minute. The oven was held at 320° C. for an additional 5 minutes. The flow rate of the carrier gas helium was 1.3 mL/minute. The MS quadrapole scanned from 50 to 550 m/z. Retention times and fragmentation patterns of product peaks were compared with authentic references to confirm peak identity.

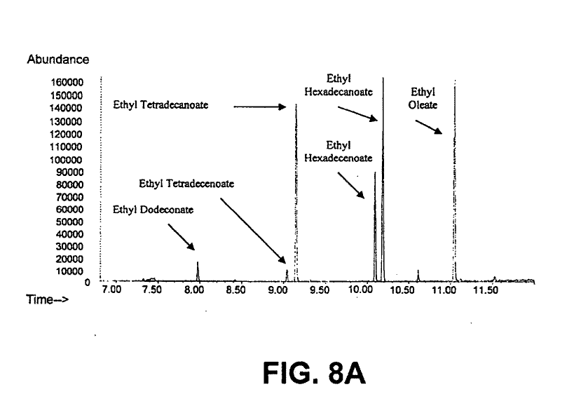

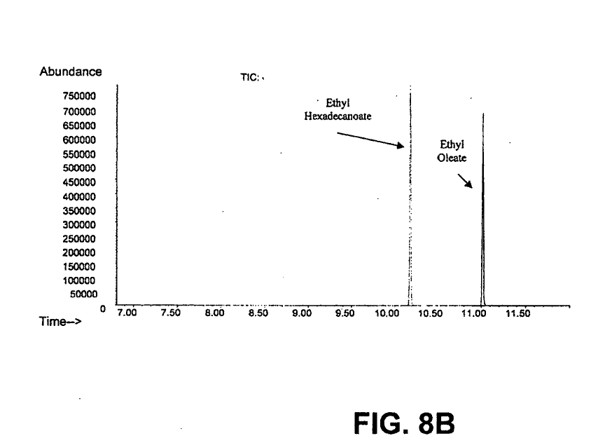

The results are presented in FIGS. 8A and 8B. For example, hexadeconic acid ethyl ester eluted at 10.18 minutes (FIG. 8A and FIG. 8B). The parent ion of 284 mass units was readily observed. More abundant were the daughter ions produced during mass fragmentation. This included the most prevalent daughter ion of 80 mass units:

Quantification was carried out by injecting various concentrations of the appropriate authentic references using the GC/MS method described above. This information was used to generate a standard curve with response (total integrated ion count) versus concentration.

### Example 9

### Ester Production from Impure Ethanol Vs. Distilled Ethanol

The present example examines the ability of a production host to create a fatty ester from an ethanol composition that contains impurities.

Plasmid pCDFDuet-1-fadD-atfA (containing sequences for ester synthase, atfA, and FadD) was transformed along with plasmid pMAL-c2X-TEchfatB3 (this plasmid is described in Table 8 and contains a nucleic acid sequence encoding thioesterase ChfatB3) into E. coli C41 (DE3ΔFadE) cells. A control strain containing the plasmids pMAL-c2X-TEchfatB3 without the thioesterase gene and pCDFDuet-1-fadD-atfA without the ester synthase gene was also made. After transformation, the resulting colonies were grown as starter cultures in M9 media supplemented with 2.0% glucose. Starter cultures were used to inoculate 80 mL fresh media. At mid log phase of growth, the cultures were induced with IPTG (1 mM final concentration). At the same time, the media was brought to 1% ethanol with either 100% ethanol or with beer (Corona, Grupo Modelo S.A. de C. V., 4.6% ethanol).

At time points 0, 19, 27, 43, 51, 73, and 115 hours, 5 mL of the culture was removed and centrifuged to separate the cells. After centrifugation, the supernatant was extracted with 1 volume of ethyl acetate. At the same time, the pellet was resuspended in M9 broth (the same volume of initial culture) and an equal volume of ethyl acetate was added to extract fatty esters. The fatty esters (C2:C14, C2:C16:1, C2:C16, C2:C18:1) were quantified by GC/MS.

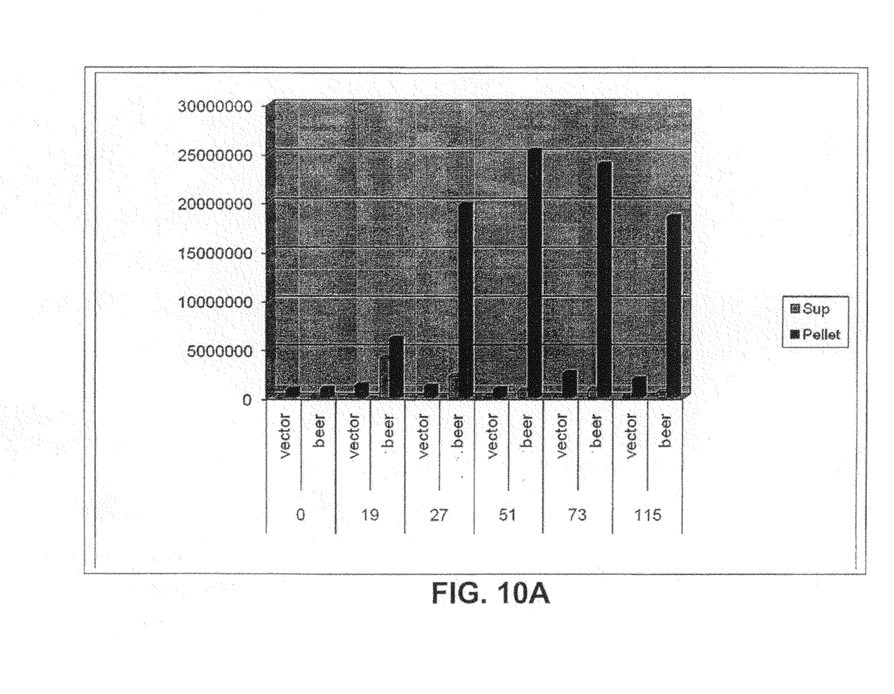

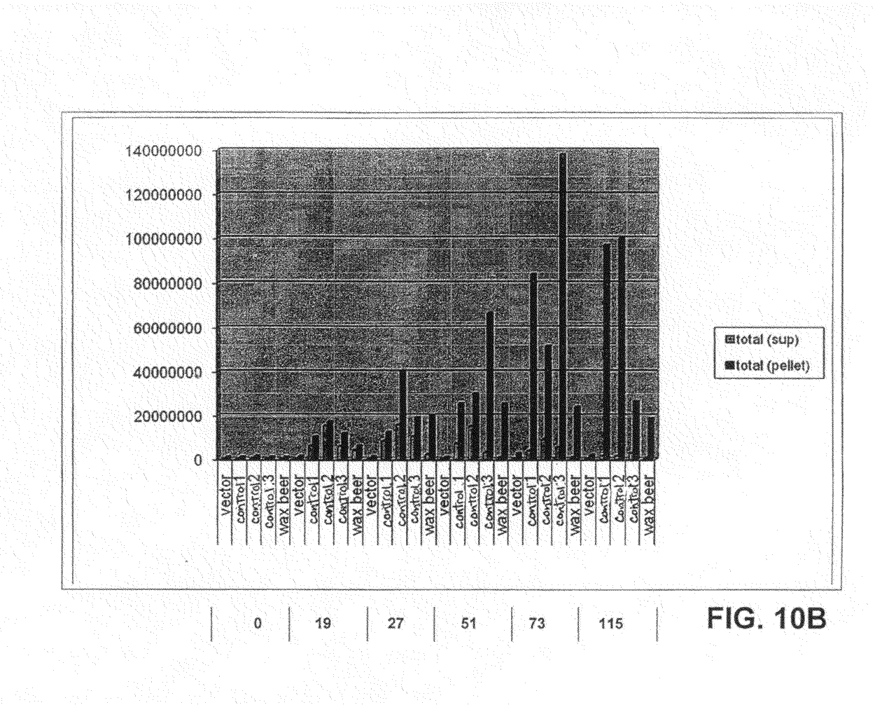

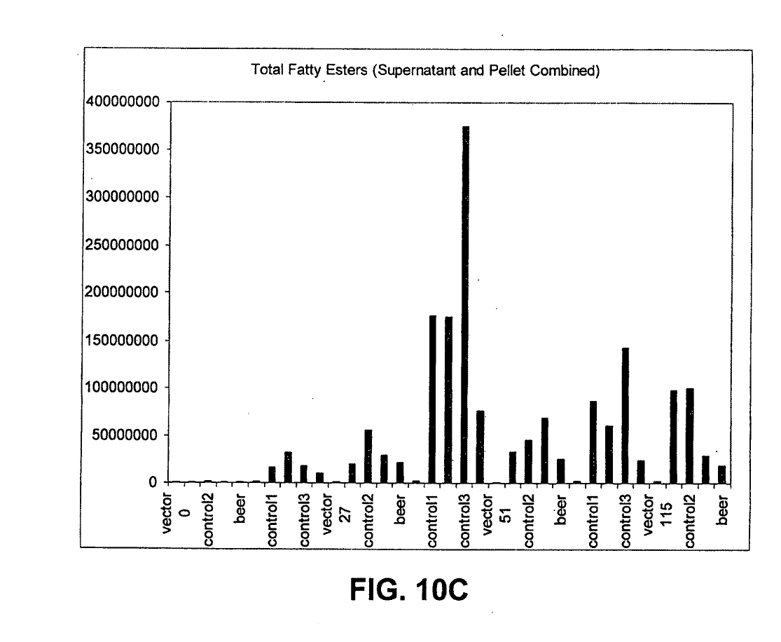

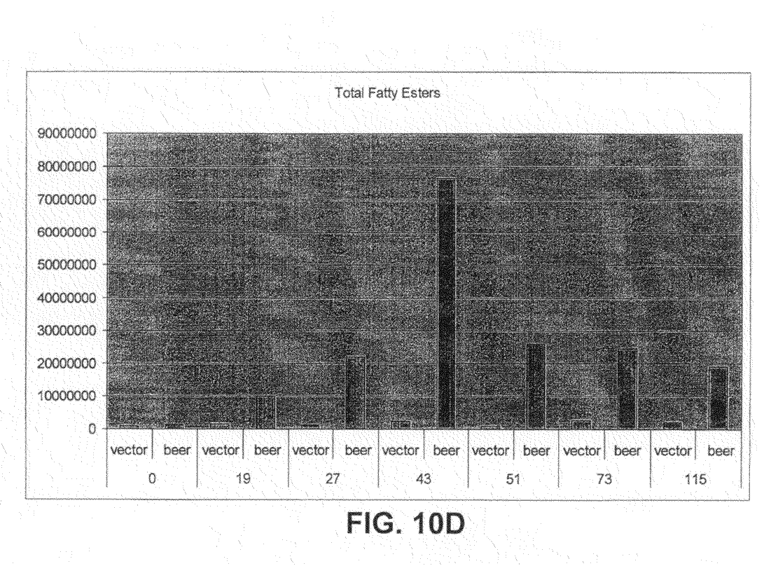

The results from the chromatograms are shown in the bar charts in FIGS. 10A and 10B over various time points (0, 19, 27, 43, 51, 73, and 115 hours, with an n of 3 for each refined ethanol batch). The ratio of total target responses of ethyl esters from the sample grown in the presence of beer compared to the control strain that was grown in the presence of 1% distilled ethanol was 4×, 17×, 29×, 40×, 9×, and 10× at time points 19, 27, 43, 51, 73, and 115, respectively. The highest target response from the beer sample was 2 fold less than the sample expressing the thioesterase and ester synthase that was grown in the presence of 1% distilled ethanol (see, FIGS. 10C and 10D, showing total fatty ester (combined sup. and pellet), including time point 43 hr). The growth of cells in the presence of beer reached stationary phase around OD₆₀₀ at 2.0 (43 hr), while the samples in the presence of distilled ethanol reached up to OD₆₀₀ 5.0.

The reported antibiotic effect of hops in beer may contribute to the more limited growth of the beer sample.

### Example 10

### Impure Alcohol in Fatty Ester Production

The present example demonstrates a method for using a volume of alcohol that includes an impurity to make a fatty ester.

An alcohol composition containing one or more impurities is obtained. The one or more impurities include one or more of acetaldehyde, acetic acid, or ethyl-lactate.

The impure alcohol composition is added to a fatty ester production vessel and combined with a fatty ester production substrate and a production host. The production host comprises a nucleic acid sequence encoding a thioesterase (EC 3.1.2.14), an ester synthase (EC 2.3.1.75), and an acyl-CoA synthase (E.C 2.3.1.86). The production host will have an attenuated acyl-CoA dehydrogenase (E.C. 1.3.99.3, 1.3.99.-) gene.

The production host is allowed to process the fatty ester production substrate and produce a fatty ester. The production host will produce a fatty ester despite the initial impurity in the alcohol composition.

### Example 11

### Impure Alcohol in Fatty Ester Production

The present example demonstrates a method for using an impure alcohol composition to make a fatty ester.

An impure alcohol composition containing an impurity is obtained. The impure alcohol composition includes at least one of the following: mannitol, cellulose, Hemicelluloses, Starch, Soluble polysaccharides, dextran, phytoglycogen, potassium, sodium, calcium, magnesium, chlorides, bicarbonate, sulfate, phosphate, iron, aluminum, silica, ammonium, nitrate, ketones, polyols, dihydroxyacetone, furfural, hydroxymethylfurfural, Amadori or Heyns products MW>1000, Amadori or Heyns products MW>200, pyrrole derivatives, pyridine derivatives, imidazole derivatives, pyrazine derivatives, heterocylcic caramel products, alicyclic caramel products, H-bonded caramel products, phenolic based colors, cis-aconitic, trans-aconitic, tartaric acid, citric acid, fumaric acid, malic acid, succinic acid, shikimic acid, 2,4-dihydroxybutyric acid, methylglyceric acid, saccharinic acids, palmitic acid, oleic acid, linoleic acid, linolenic acid, acetic acid, lactic acid, formic acid, glyceric acid, oxalic acid, glycolic acid, aromatic acids, ferulic acid, p-hydroxybenzoic acid, vanillic acid, caffeic acid, p-coumaric acid, 3,4-dihydroxybenzoic acid, 2,3-dihydroxybenzoic acid, phenolics, lignin, chlorogenic acid, neutral phenolics, glycosidic flavinoids, Swertisin, luteolins, 6-methoxyluteolin, apigenins, tricins, fats, phosphatides, chlorophyll a & b, carotene, xanthophyll, anthocyanins, phosphatidylethanolamine, lecithin, vitamins, thiamine, riboflavin, pyridoxine (b6 group), niacin, calcium pantothenate, biotin, folic acid, betaine, amides, acetamide, lactamide, n-sugar color, pyrollidone carboxylic acid (pea), allantoin, allantoic acid, aspartic acid, asparagine, asparagine, glutamic acid, glutamine, glutamine, α-alanine, valine, γ-aminobutyric acid, threonine, isoleucine, glycine, leucine, lysine, serine, arginine, phenylalanine, tyrosine, histidine, hydroxyproline, proline, methionine, tryptophan, uridine, adenine, pesticides and herbicides, aldrin, dieldrin, and chlordane, trehalose; acetaldehyde, acetals; 3-methyl-1-butanol, 2-methyl-1-propanol (isobutanol), 2-propanol, 1-propanol, 1-butanol, 2-methylbutanol, sulfite waste liquor, fusel alcohols, n-pentanol, n-hexanol, n-heptanol, higher straight-chain aldehydes, pentanal, hexanal, heptanal, and octanal, aromatic alcohols, phenol derivatives, mannitol, silica, maillard, caramel color, organic acids (C4-C6), organics acids (C2-C3), aromatic acids, phenolics/lignins, vitamins, other n species, polypeptides N>2, nucleic acids, fructose, iso-maltose, acetic acid, trehalose (e.g., 200 to 400 ppm of trehalose and 50 to 200 ppm isomaltose), sorbitol, erythritol, and mannitol (e.g., concentrations of 5 to 50 ppm), glycerol, Lactobacillus, lactic acid, yeast, succinic acid, and acetic acid in a detectable amount.

The impure alcohol composition is added to a fatty ester production vessel and combined with a fatty ester production substrate and a fatty ester production host. The fatty ester production host is an E. coli bacterium comprising a nucleic acid sequence encoding a thioesterase (EC 3.1.2.14), an ester synthase (EC 2.3.1.75), and an acyl-CoA synthase (E.C.2.3.1.86), and, optionally, having an attenuated acyl-CoA dehydrogenase (E.C. 1.3.99.3, 1.3.99.-) gene.

The production host is allowed to process the production substrate and produce a fatty ester. The production host produces a fatty ester despite the initial impurity in the alcohol composition.

### Example 12

### Unrefined Ethanol in Fatty Ester Production

The present example demonstrates a method for using an ethanol composition that includes an impurity to make a fatty ester.

An unrefined ethanol composition is obtained. The unrefined ethanol composition includes yeast. The unrefined ethanol composition is added to a fatty ester production vessel and is combined with a fatty ester production host and carbon source for the fatty ester production host. The production host is allowed to process the carbon source and produce a fatty ester by using the available ethanol. The production host is an E. coli bacterium comprising a nucleic acid sequence encoding a thioesterase (EC 3.1.2.14), an ester synthase (EC 2.3.1.75), and an acyl-CoA synthase (E.C.2.3.1.86). The production host, optionally, has an attenuated acyl-CoA dehydrogenase (E.C. 1.3.99.3, 1.3.99.-) gene. The production host produces a fatty ester despite the initial yeast impurity in the ethanol composition.

### Example 13

### Impure Isopropanol in Fatty Ester Production

The present example demonstrates a method for using an impure isopropanol composition that includes an impurity to make a fatty ester.

An isopropanol composition having an impurity is obtained. The impure isopropanol composition is added to a fatty ester production vessel and combined with a fatty ester production substrate and a production host. The production host comprises a nucleic acid sequence encoding a thioesterase (EC 3.1.2.14), an ester synthase (EC 2.3.1.75), and an acyl-CoA synthase (E.C.2.3.1.86), and, optionally, lacks a nucleic acid sequence encoding an acyl-CoA dehydrogenase (E.C. 1.3.99.3, 1.3.99.-).

The production host is allowed to process the production substrate and produce a fatty ester. The production host produces a fatty ester despite the initial impurity in the isopropanol composition.

As will be appreciated by one of skill in the art, the above example can be used with alternative production hosts, such as a production host that includes an exogenous nucleic acid sequence encoding a thioesterase (EC 3.1.2.14), an ester synthase (EC 2.3.1.75), an alcohol acetyltransferase (2.3.1.84), a fatty alcohol forming acyl-CoA reductase (1.1.1.*), an acyl-CoA reductase (EC 1.2.1.50), or an alcohol dehydrogenase (EC 1.1.1.1). In addition, the isopropanol can be replaced with other impure alcohols, such as propanol or longer alcohols (e.g., 4, 5, 6, 7, or 8 carbon alcohols). Furthermore, alternative impurities can also be present.

### Example 14

### Determination of Impact of Alcohol Impurity and Remedial Measures Therefor

The present example demonstrates a method for determining if an impurity in an alcohol composition adversely impacts the fatty ester production host and hinders fatty ester production.

The impure alcohol composition is combined with a production media and is combined with an E. coli bacterium. The E. coli bacterium has a nucleic acid sequence encoding a thioesterase (EC 3.1.2.14), an ester synthase (EC 2.3.1.75), and an acyl-CoA synthase (E.C.2.3.1.86). The E. coli bacterium, optionally, lacks a nucleic acid sequence encoding an acyl-CoA dehydrogenase (or the bacterium is modified to express a reduced level of acyl-CoA dehydrogenase). One then looks for the survival of the E. coli bacterium and whether fatty esters are produced. One also determines the amount of fatty ester production in comparison to fatty ester production in the same bacterium in a similar production system that utilizes the refined form of the alcohol.

As will be appreciated by one of skill in the art, by performing the assay in this Example, one can determine if any impurity will adversely impact any of the production hosts. As will be appreciated by one of skill in the art, in light of the present disclosure, if a specific amount of an impurity does adversely impact the fatty ester production, one can dilute the alcohol sample with water and then combine it with the production host. The production host will then be allowed to process the diluted alcohol and production substrate to produce a fatty ester. In some embodiments, one determines the minimal inhibitory concentration (MIC) of the pure compound as compared to the impure compounds. If the impure compound has a lower MIC, then there can be one or more compounds in the mix that are inhibitory relative to the pure compound. These compounds can either be removed or the mixture diluted.

### Example 15

### Serial Ethanol and Fatty Ester Production

The present example demonstrates how an impure or unrefined ethanol composition can be used in a production system to produce fatty esters. The ethanol composition is produced serially and before fatty ester production.

In an ethanol production vessel, an ethanol composition is produced by using yeast and an appropriate yeast substrate under the appropriate fermentation conditions. The ethanol composition production occurs in a liquid environment.

After a sufficient amount of time has passed to allow the yeast to produce a desired amount of ethanol in the ethanol composition, at least some of the ethanol composition is moved from the ethanol production vessel to a fatty ester production vessel. The product is not distilled prior to being added to the fatty ester production vessel, although it can be filtered.

The fatty ester production host, contained in the fatty ester production vessel, is then allowed to process the ethanol and a fatty ester production substrate, thereby converting the ethanol to a fatty ester. The fatty ester will be produced despite the presence of impurities due to the unrefined nature of the ethanol.

As will be appreciated by one of skill in the art, in some embodiments, the method in the above example can be modified. In some embodiments, the ethanol composition is filtered to remove the yeast prior to being placed into the fatty ester production vessel. The ethanol and fatty ester production can occur at the same production facility or at separate production facilities (e.g., the ethanol can be sourced from a third party ethanol production facility). The liquid from the ethanol production vessel is moved to the fatty ester production vessel by flowing the liquid through a piping system directly connecting the ethanol production vessel to the fatty ester production vessel. In some other embodiments, the liquid is moved between the ethanol production vessel and the fatty ester production while stored in containers.

### Example 16

### Continuous Serial Production Vessel Arrangement

The present example demonstrates how an impure or unrefined alcohol composition can be used in a production system to produce fatty esters. An alcohol composition is produced and continually transferred to a fatty ester production vessel. The production of the alcohol composition is continuous for a period of time and the transfer of the alcohol composition to the fatty ester production vessel is also continuous for at least part of that production period.

In an alcohol production vessel, an alcohol composition is produced by an alcohol production host and an appropriate alcohol production substrate under the appropriate production conditions. The alcohol composition production can occur in a liquid environment.

As the alcohol composition is produced by the alcohol production host the production media alcohol composition is continuously transferred to a fatty ester production vessel. The fatty ester production vessel includes a fatty ester production substrate and a fatty ester production host. The alcohol composition is not substantially refined prior to being added to the fatty ester production vessel. The temperature and other environmental conditions of the alcohol production vessel are optimized for alcohol production. The temperature and other environmental conditions of the fatty ester production vessel are optimized for fatty ester production. The alcohol production vessel is kept at a lower temperature than the fatty ester production vessel. The solution inside of the fatty ester vessel is kept at a higher pH (in comparison to the solution in the alcohol production vessel).

The fatty ester production host, contained in the fatty ester production vessel, is allowed to process the alcohol composition and a fatty ester production substrate, thereby producing fatty ester. The fatty ester will be produced despite the presence of impurities due to the impure nature of the alcohol.

### Example 17

### Serial Alcohol and Fatty Ester Production

The present example demonstrates how an impure or unrefined source of alcohol can be used in a production system to produce fatty esters. An alcohol composition is produced serially, before fatty ester production.

An alcohol composition is produced in an alcohol production vessel. The alcohol composition is produced by an alcohol production host that converts an alcohol production substrate into an alcohol. Once the alcohol composition is produced, at least a part of the alcohol composition from the alcohol production vessel, which will include the alcohol, is transported to a fatty ester production vessel and combined with a fatty ester production substrate and a fatty ester production host. The alcohol composition is below 20% alcohol when it is placed into the fatty ester production vessel.

The fatty ester production host comprises a nucleic acid sequence encoding a thioesterase (EC 3.1.2.14), an ester synthase (EC 2.3.1.75), and an acyl-CoA synthase (E.C.2.3.1.86). The fatty ester production host has an attenuated or deleted acyl-CoA dehydrogenase (E.C. 1.3.99.3, 1.3.99.-) gene.

The fatty ester production host is allowed to process the fatty ester production substrate and produce a fatty ester. The fatty ester production host will produce a fatty ester despite the fact that the alcohol entering the fatty ester production vessel has not been distilled.

### Example 18

### Parallel Alcohol and Fatty Ester Production

### Single Production Vessel

The present example demonstrates how an impure or unrefined alcohol composition can be used in a production system to produce fatty esters. Alcohol and fatty ester are simultaneously produced in the same production vessel.

An alcohol composition is produced in a production vessel via an alcohol production host processing a production substrate. The alcohol production host will be allowed to convert the production substrate into an alcohol.

Within the same production vessel is a fatty ester production host. As the alcohol is produced by the alcohol production host, it will be used by the fatty ester production host, along with a production substrate present in the vessel, to produce a fatty ester. The fatty ester production host will produce a fatty ester despite the fact that the vessel contains an alcohol production host and side products from the production of the alcohol.

As will be appreciated by one of skill in the art, the variables in the above Example can be adjusted for various embodiments. In some embodiments, a single production substrate is used for two different production hosts. In some embodiments, two different production substrates are used for two different production hosts. In some embodiments, the environmental conditions (e.g., temperature and pH) for the production of the alcohol and the fatty ester are optimized for the production of the fatty ester, optimized for the production of the alcohol, or set for conditions between the optimal conditions for each of the two processes. In some embodiments, the environmental conditions are cycled between those that favor alcohol production and those that favor fatty ester production.

### Example 19

### Parallel Ethanol and Fatty Ester Production

### Single Production Vessel

The present example demonstrates how an impure or unrefined source of ethanol can be used in a production system to produce fatty esters. Ethanol and fatty esters are produced simultaneously in the same vessel.

Ethanol is produced in a production vessel via an ethanol production host processing an ethanol production substrate. The ethanol production host will be yeast and the production substrate will be a sugar, such as glucose. The yeast will be allowed to convert the sugar into ethanol. Within the same production vessel will be a fatty ester production host. The fatty ester production host comprises a nucleic acid sequence encoding a thioesterase (EC 3.1.2.14), an ester synthase (EC 2.3.1.75), and an acyl-CoA synthase (E.C.2.3.1.86). The fatty ester production host will lack or have an attenuated acyl-CoA dehydrogenase (E.C. 1.3.99.3, 1.3.99.-).

As the ethanol is produced by the yeast, it will be used by the fatty ester production host, along with the sugar present in the vessel, to produce a fatty ester. The fatty ester production host will produce a fatty ester despite the fact that the vessel contains an ethanol production host and by-products from the production of the ethanol.

### Example 20

### Parallel Alcohol and Fatty Ester Production

### Single Production Vessel

The present example demonstrates how an impure or unrefined source of alcohol can be used in a production system to produce fatty esters. Alcohol and fatty esters are produced in the same vessel.

Alcohol is produced in a production vessel containing Zymomonas mobilis that has been modified to utilize 5 carbon sugars, but not 6 carbon sugars.

Within the same production vessel is a fatty ester production host (any of the production hosts disclosed herein). The fatty ester production host preferentially converts 6 carbon sugars over 5 carbon sugars.

The product media includes a mix of 5 and 6 carbon sugars so as to support both ethanol production (using the Z. mobilis) and fatty ester production (using E. coli that preferentially processes 6 carbon sugars). The quantity of each type of sugar is regulated to achieve similar growth rates in the Z. mobilis and E. coli. In the event that 4 moles of glucose give 1 mole of fatty ester, the media can be supplemented with 1 mole of pentose sugars to give 1 mole of ethanol. In this way, the health of both the E. coli and the Z. mobilis are ensured (as they both grow at similar pH and temperature), yet one does not outgrow the other because they feed off of different sugars.

As disclosed earlier, as the alcohol is produced by the alcohol production host, it is used by the fatty ester production host, along with the production substrate present in the vessel, to produce a fatty ester. The fatty ester production host produces a fatty ester despite the fact that the vessel contains an alcohol production host and side products from the production of the alcohol.

As will be appreciated by one of skill in the art, this Example can be especially advantageous for a production substrate that includes corn-based carbon source (e.g., corn kernels or corn cobs), as the resultant mixture contains some 5 carbon sugars, but is rich in 6 carbon sugars. In addition, other sources of 5 and 6 carbon sugars can be used.

In some embodiments, the production system utilizes a modified E. coli that has been modified to efficiently process 5 carbon sugars (but does not effectively process 6 carbon sugars), while the Zymomas or yeast production host efficiently processes 6 carbon sugars (but not 5 carbon sugars). The growth of the fatty ester and alcohol production hosts are regulated by the supply of 5 and 6 carbon sugars, respectively. References providing further guidance regarding various individual aspects of the above can be found in “Evaluation of Zymomonas-based ethanol production from a hydrolysed waste starch stream,” Linda Davis, Peter Rogers John Pearce and Paul Peiris Biomass and Bioenergy, 30:809-814, 2006; “Evaluation of wheat stillage for ethanol production by recombinant Zymomonas mobilis,” Linda Davis, Young-Jae Jeon, Charles Svenson, Peter Rogers, John Pearce, Paul Peiris, Biomass and Bioenergy, 29:49-59, 2005; and “Effects of lignocellulose degradation products on ethanol fermentations of glucose and xylose by Saccharomyces cerevisiae, Zymomonas mobilis, Pichia stipitis, and Candida shehatae,” J. P. Delgenes, R. Moletta, J. M. Navarro, Enzyme and Microbial Technology, 19:220-225, 1996 (the entireties of each of which is incorporated by reference). In some embodiments, the production host can be one or more of Pichia stipitis, Candida shehatae, and Kluyveromyces fragilis NRRL 665 (discussed in greater detail in “Ethanol production from lactose by coculture of Kluyveromyces fragilis and Zymomonas mobilis,” Numbi Ramudu Kamini, Paramasamy Gunasekaran, Journal of Fermentation and Bioengineering, 68:305-309, 1989, the entirety of each of which is incorporated by reference).

### Example 21

### Production of Biodiesel

The present example outlines how the fatty ester products can be further processed for use as a biodiesel.

The fatty ester product from any of the above fatty ester producing examples can be collected as outlined in Example 7. After the hydrophobic phase is recovered, the fatty esters can be further purified and concentrated if desired. In addition, various specific types of fatty esters can be isolated or concentrated as desired. The recovered fatty ester composition can then be refined to at least about 90% fatty esters. In some cases, the collected fatty ester composition can be purified to be at least about 99% fatty esters. The concentrated product can then be used as a biodiesel fuel product for various diesel engines (e.g., as the combustible fuel in combustion engines in vehicles).

In another embodiment, the fatty esters can be combined with a different biodiesel, petroleum-based diesel, or other fuel additives well known in the art.

In this disclosure, the use of the singular can include the plural unless specifically stated otherwise or unless, as will be understood by one of skill in the art in light of the present disclosure, the singular is the only functional embodiment. Thus, for example, “a” can mean more than one, and “one embodiment” or “one example” can mean that the description applies to multiple embodiments. The phrase “and/or” denotes a shorthand way of indicating that the specific combination is contemplated in combination and, separately, in the alternative.

The section headings used herein are for organizational purposes only and are not to be construed as limiting the described subject matter in any way.

It will be appreciated that there is an implied “about” prior to the temperatures, concentrations, times, etc. discussed in the present teachings, such that slight and insubstantial deviations are within the scope of the present teachings herein. For example, “a primer” means that more than one primer can, but need not, be present. For example, but without limitation, one or more copies of a particular primer species, as well as one or more versions of a particular primer type, for example, but not limited to, a multiplicity of different forward primers can be present. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the invention.

### Incorporation by Reference

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including, but not limited to defined terms, term usage, described techniques, or the like, this application controls.

### EQUIVALENTS

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

We claim:

1. A method of making a fatty ester, comprising:
processing an alcohol production substrate to produce an alcohol composition that comprises an alcohol;
providing the alcohol composition, without refining the alcohol composition, to a fatty ester production host;
providing a fatty ester production substrate to the fatty ester production host; and
processing the fatty ester production substrate in the presence of the alcohol to produce a fatty ester.

2. The method of claim 1, wherein, between processing the alcohol production substrate and providing the alcohol composition to the fatty ester production host, an alcohol concentration of the alcohol composition, as measured by volume, changes by no more than about 20%.

3. The method of claim 1, wherein the alcohol is less than about 20% by volume of the alcohol composition that is provided to the fatty ester production host.

4. The method of claim 3, wherein the alcohol is less than about 95% by volume of the alcohol composition that is provided to the fatty ester production host.

5. The method of claim 1, wherein processing the alcohol production substrate and processing the fatty ester production substrate are performed simultaneously.

6. The method of claim 5, wherein processing the alcohol production substrate is performed in a first vessel and processing the fatty ester production substrate is performed in a second vessel,
wherein the first vessel is in fluid communication with the second vessel.

7. The method of claim 1, wherein the alcohol comprises ethanol.

8. The method of claim 1, wherein the alcohol comprises an aliphatic group having at least 1 carbon atom.

9. The method of claim 8, wherein the aliphatic group includes a number of carbon atoms, wherein the number of carbon atoms is selected from the group consisting of: 3, 4, 5, 6, 7, 8, 9, 10, 11, 12, 13, 14, 15, and 16.

10. The method of claim 8, wherein the alcohol comprises an alkyl group having at least 2 carbon atoms.

11. The method of claim 8, wherein the alcohol is chosen from the group consisting of ethanol, propanol, isopropanol, butanol, isobutanol, pentanol, isoamyl alcohol, isopentenol, hexanol, heptanol, octanol, nonanol, decanol, geraniol, undecanol, dodecanol, tetradecanol, pentadecanol, farnesol, and any combination thereof.

12. The method of claim 1, wherein the fatty ester production host is selected from the group consisting of at least one of the following: a mammalian cell, plant cell, insect cell, yeast cell, fungus cell, filamentous fungi cell, bacterial cell, a Gram-positive bacteria, a Gram-negative bacteria, the genus Escherichia, the genus Bacillus, the genus Lactobacillus, the genus Rhodococcus, the genus Pseudomonas, the genus Aspergillus, the genus Trichoderma, the genus Neurospora, the genus Fusarium, the genus Humicola, the genus Rhizomucor, the genus Kluyveromyces, the genus Pichia, the genus Mucor, the genus Myceliophtora, the genus Penicillium, the genus Phanerochaete, the genus Pleurotus, the genus Trametes, the genus Chrysosporium, the genus Saccharomyces, the genus Stenotrophamonas, the genus Schizosaccharomyces, the genus Yarrowia, the genus Streptomyces, a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, a Mucor michei cell, a Streptomyces lividans cell, a Streptomyces murinus cell, an Actinomycetes cell, a CHO cell, a COS cell, a VERO cell, a BHK cell, a HeLa cell, a Cv1 cell, an MDCK cell, a 293 cell, a 3T3 cell, a PC12 cell, an E. coli cell, a strain B E. coli cell, a strain C E. coli cell, a strain K E. coli cell, and a strain W E. coli cell.

13. The method of claim 1, wherein the fatty ester production host comprises a recombinant cell.

14. The method of claim 13, wherein the recombinant cell lacks a nucleic acid sequence encoding an acyl-CoA dehydrogenase enzyme or wherein expression of an acyl-CoA dehydrogenase enzyme is attenuated in the recombinant cell.

15. The method of claim 13, wherein the recombinant cell comprises a heterologous nucleic acid sequence encoding an ester synthase enzyme.

16. The method of claim 13, wherein the recombinant cell comprises a heterologous nucleic acid sequence encoding a thioesterase enzyme.

17. The method of claim 13, wherein the recombinant cell comprises a heterologous nucleic acid sequence encoding an acyl-CoA synthase enzyme.

18. The method of claim 1, wherein processing the alcohol production substrate is performed by an alcohol production host.

19. The method of claim 1, wherein processing the alcohol production substrate is performed by an alcohol production host selected from the group consisting of at least one of the following: Saccharomyces cerevisiae, Saccharomyces distaticus, Saccharomyces uvarum, Schizosaccharomyces pombe, Kluyveromyces marxianus, Kluyveromyces fragilis, Candida pseudotropicalis, Candida brassicae, the genus Clostridium, Clostridium acetobutylicum, Clavispora lusitaniae, Clavispora opuntiae, Pachysolen tannophilus, Bretannomyces clausenii, Zymomonas mobilis, the genus Zymomonas, Clostridium thermocellum, Klebsiella oxytoca, Bacillus subtillis, yeast, the genus Saccharomyces, the genus Thermaloga, the genus Bacillus, the genus Pseudomonas, the genus Actinomycetes, the genus Streptomyces, the genus Escherichia the genus Kluyveromyces, the genus Candida, the genus Clavispora, the genus Pichia, the genus Schizosaccharomyces, the genus Hansenula, the genus Pachysolen, and the genus Bretannomyces.

20. The method of claim 1, wherein the alcohol production substrate comprises glucose.

21. The method of claim 1, wherein the fatty ester production substrate comprises glucose.

22. The method of claim 1, wherein the alcohol production substrate and the fatty ester production substrate both comprise glucose.

23. The method of claim 1, wherein the alcohol production substrate and the fatty ester production substrate are selected from the group consisting of at least one of the following: monosaccharide, glucose, fructose, mannose, galactose, oligosaccharide, fructo-oligosaccharide, galacto-oligosaccharide, polysaccharide, xylose, arabinose, disaccharide, sucrose, maltose, turanose, cellulosic material, methyl cellulose, sodium carboxymethyl cellulose, succinate, lactate, acetate, starch derivatives, lignocellulosic biomass, and any combination thereof.

24. The method of claim 1, wherein processing the alcohol production substrate, providing the alcohol composition, providing the fatty ester production substrate, and processing the fatty ester production substrate are performed in the order in which these steps are presented in claim 1.

25. The method of claim 1, wherein processing the alcohol production substrate and processing the fatty ester production substrate are performed in a same vessel.

26. A method of making a fatty ester, comprising:
providing an alcohol composition to a fatty ester production host, wherein the alcohol composition contains less than about 20% alcohol by volume immediately prior to being provided to the fatty ester production host;
adding a fatty ester production substrate to the fatty ester production host; and
processing the fatty ester production substrate in the presence of the alcohol composition to produce a fatty ester.

27. The method of claim 26, wherein the alcohol composition comprises at least one alcohol selected from the group consisting of: ethanol, propanol, isopropanol, butanol, isobutanol, pentanol, isoamyl alcohol, isopentenol, hexanol, heptanol, octanol, nonanol, decanol, geraniol, undecanol, dodecanol, tetradecanol, pentadecanol, farnesol, and any combination thereof.

28. The method of claim 26, wherein the alcohol composition comprises at least one alcohol of the formula: R₁—OH, wherein R₁ is a saturated carbon chain.

29. The method of claim 26, wherein the alcohol composition comprises at least one alcohol of the formula: R₁—OH, wherein R₁ comprises an unsaturated carbon chain.

30. The method of claim 26, wherein the alcohol composition comprises at least one alcohol of the formula: R₁—OH, wherein R₁ comprises a branched carbon chain.

31. The method of claim 26, wherein the alcohol composition comprises at least one alcohol of the formula: R₁—OH, wherein R₁ comprises an alkyl carbon chain.

32. The method of claim 26, wherein the alcohol composition comprises at least one alcohol of the formula: R₁—OH, wherein R₁ comprises a straight chain alcohol.

33. The method of claim 26, wherein the alcohol composition comprises at least one alcohol of the formula: R₁—OH, wherein R₁ is an aliphatic group comprising a number of carbon atoms selected from the group consisting of 1, 2, 3, 4, 5, 6, 7, 8, 9, 10, 11, 12, 13, 14, 15, 16, 17, 18, 19, and 20.

34. The method of claim 26, wherein the alcohol composition further comprises an alcohol production host.

35. The method of claim 34, wherein the alcohol production host is selected from the group consisting of at least one of the following: Saccharomyces cerevisiae, Saccharomyces distaticus, Saccharomyces uvarum, Schizosaccharomyces pombe, Kluyveromyces marxianus, Kluyveromyces fragilis, Candida pseudotropicalis, Candida brassicae, the genus Clostridium, Clostridium acetobutylicum, Clavispora lusitaniae, Clavispora opuntiae, Pachysolen tannophilus, Bretannomyces clausenii, Zymomonas mobilis, the genus Zymomonas, Clostridium thermocellum, Klebsiella oxytoca, Bacillus subtillis, yeast, the genus Saccharomyces, the genus Thermatoga, the genus Bacillus, the genus Pseudomonas, the genus Actinomycetes, the genus Streptomyces, the genus Escherichia, the genus Kluyveromyces, the genus Candida, the genus Clavispora, the genus Pichia, the genus Schizosaccharomyces, the genus Hansenula, the genus Pachysolen, the genus Bretannomyces, and combinations thereof.

36. The method of claim 26, wherein the fatty ester production substrate is provided in the alcohol composition.

37. The method of claim 26, wherein the alcohol composition comprises an alcohol production substrate.

38. A production system, comprising:
a fatty ester production vessel;
a fatty ester production host; and
a source of impure alcohol in fluid communication with the fatty ester production vessel.

39. The production system of claim 38, wherein the fatty ester production host comprises a heterologous nucleic acid sequence encoding a thioesterase.

40. The production system of claim 38, wherein the fatty ester production host comprises a heterologous nucleic acid sequence encoding an ester synthase.

41. The production system of claim 38, wherein the fatty ester production host comprises a heterologous nucleic acid sequence encoding an acyl-CoA synthase.

42. The production system of claim 38, wherein the fatty ester production host has attenuated acyl-CoA dehydrogenase activity.

43. The production system of claim 38, wherein the fatty ester production host lacks an acyl-CoA dehydrogenase gene.

44. The production system of claim 38, wherein the fatty ester production vessel comprises a fatty ester production host comprising a heterologous nucleic acid sequence encoding an enzyme selected from the group consisting of: a thioesterase, an ester synthase, an alcohol acyltransferase, a fatty alcohol forming acyl-CoA reductase, an acyl-CoA reductase, an alcohol dehydrogenase, and combinations thereof.

45. The production system of claim 38, comprising an alcohol production vessel.

46. The production system of claim 45, wherein the production system is configured to feed the source of impure alcohol from the alcohol production vessel to the fatty ester production vessel.

47. The production system of claim 45, wherein the fatty ester production vessel and the alcohol production vessel are a same vessel.

48. The production system of claim 45, further comprising a production substrate storage unit in fluid communication with the fatty ester production vessel.

49. The production system of claim 48, wherein the production substrate is selected from the group consisting of monosaccharide, glucose, fructose, mannose, galactose, oligosaccharide, fructo-oligosaccharide, galacto-oligosaccharide, polysaccharide, xylose, arabinose, disaccharide, sucrose, maltose, turanose, cellulosic material, methyl cellulose, sodium carboxymethyl cellulose, saturated or unsaturated fatty acid ester, succinate, lactate, acetate, starch derivatives, lignocellulosic biomass, and any combination thereof.

50. The production system of claim 48, wherein the production substrate storage unit is in fluid communication with the alcohol production vessel.

51. The production system of claim 38, wherein the source of impure alcohol comprises an alcohol composition that is between about 1% to about 25% alcohol by volume.

52. The production system of claim 51, wherein the alcohol composition comprises an alcohol selected from the group consisting of: ethanol, propanol, isopropanol, butanol, isobutanol, pentanol, isoamyl alcohol, isopentenol, hexanol, heptanol, octanol, nonanol, decanol, geraniol, undecanol, dodecanol, tetradecanol, pentadecanol, farnesol, and any combination thereof.

53. The production system of claim 52, wherein the alcohol composition comprises ethanol.

54. The production system of claim 51, wherein the alcohol composition comprises at least one alcohol of the formula: R₁—OH, wherein R₁ is an aliphatic group comprising a number of carbon atoms selected from the group consisting of 1, 2, 3, 4, 5, 6, 7, 8, 9, 10, 11, 12, 13, 14, 15, 17, 18, 19, and 20.

55. A production system, comprising:
a production substrate feed source;
an ethanol production vessel comprising an ethanol production host, wherein the ethanol production vessel is in fluid communication with the production substrate feed source; and
a fatty ester production vessel comprising a fatty ester production host, wherein the fatty ester production vessel is in fluid communication with the production substrate feed source, and wherein the fatty ester production host comprises a heterologous nucleic acid sequences encoding a thioesterase, an ester synthase, and an acyl-CoA synthase.

56. The production system of claim 55, wherein the fatty ester production host lacks a nucleic acid sequence encoding an acyl-CoA dehydrogenase enzyme or wherein the fatty ester production host expresses a nucleic acid encoding an acyl-CoA dehydrogenase enzyme at an attenuated level.

57. The production system of claim 55, wherein the ethanol production vessel and the fatty ester production vessel are a same vessel.

58. The production system of claim 55, wherein the ethanol production vessel is separate from and in fluid communication with the fatty ester production vessel, and wherein the ethanol production vessel is configured to produce and feed an ethanol product to the fatty ester production vessel.

59. The production system of claim 55, wherein the ethanol production vessel comprises an ethanol production substrate comprising a material selected from the group consisting of: monosaccharide, glucose, fructose, mannose, galactose, oligosaccharide, fructo-oligosaccharide, galacto-oligosaccharide, polysaccharide, xylose, arabinose, disaccharide, sucrose, maltose, turanose, cellulosic material, methyl cellulose, sodium carboxymethyl cellulose, succinate, lactate, acetate, starch derivatives, lignocellulosic biomass, and any combination thereof.

60. A fatty ester composition comprising:
an alcohol production host; and
a fatty ester, wherein the fatty ester is at least 40% by volume of the fatty ester composition.

61. The fatty ester composition of claim 60, wherein the alcohol production host is a yeast.

62. The fatty ester composition of claim 61, wherein the yeast is Saccharomyces cerevisiae.

63. The fatty ester composition of claim 60, wherein the alcohol production host is selected from the group consisting of Saccharomyces cerevisiae, Saccharomyces distaticus, Saccharomyces uvarum, Schizosaccharomyces pombe, Kluyveromyces marxianus, Kluyveromyces fragilis, Candida pseudotropicalis, Candida brassicae, the genus Clostridium, Clostridium acetobutylicum, Clavispora lusitaniae, Clavispora opuntiae, Pachysolen tannophilus, Bretannomyces clausenii, Zymomonas mobilis, the genus Zymomonas, Clostridium thermocellum, Klebsiella oxytoca, Bacillus subtillis, yeast, the genus Saccharomyces, the genus Thermatoga, the genus Bacillus, the genus Pseudomonas, the genus Actinomycetes, the genus Streptomyces, the genus Escherichia, the genus Kluyveromyces, the genus Candida, the genus Clavispora, the genus Pichia, the genus Schizosaccharomyces, the genus Hansenula, the genus Pachysolen, the genus Bretannomyces, and combinations thereof.

64. The fatty ester composition of claim 60, wherein the alcohol production host is a bacterium.

65. The fatty ester composition of claim 64, wherein the bacterium is from a genus chosen from the group consisting of the genus Zymomonas, the genus Clostridium, and the genus Escherichia.

66. The fatty ester composition of claim 65, wherein the bacterium is Escherichia coli.

67. The fatty ester composition of claim 60, wherein the fatty ester is an ethyl ester.

68. The fatty ester composition of claim 60, further comprising:
a detectable amount of at least one impurity selected from the group consisting of: mannitol, cellulose, hemicelluloses, starch, soluble polysaccharides, dextran, phytoglycogen, potassium, sodium, calcium, magnesium, chlorides; bicarbonate, sulfate, phosphate, iron, aluminum, silica, ammonium, nitrate, ketones, polyols, dihydroxyacetone, furfural, hydroxymethylfurfural, Amadori products, Heyns products, pyrrole derivatives, pyridine derivatives, imidazole derivatives, pyrazine derivatives, heterocylcic caramel products, alicyclic caramel products, H-bonded caramel products, phenolic based colors, cis-aconitic acid, trans-aconitic acid, tartaric acid, citric acid, fumaric acid, malic acid, succinic acid, shikimic acid, 2,4-dihydroxybutyric acid, methylglyceric acid, saccharinic acids, palmitic acid, oleic acid, linoleic acid, linolenic acid, acetic acid, lactic acid, formic acid, glyceric acid, oxalic acid, glycolic acid, aromatic acids, ferulic acid, p-hydroxybenzoic acid, vanillic acid, caffeic acid, p-coumaric acid, 3,4-dihydroxybenzoic acid, 2,3-dihydroxybenzoic acid, phenolics, lignin, chlorogenic acid, neutral phenolics, glycosidic flavinoids, luteolins, 6-methoxyluteolin, apigenins, tricins, fats, phosphatides, chlorophyll A, chlorophyll B, carotene, xanthophyll, anthocyanins, phosphatidylethanolamine, lecithin, vitamins, thiamine, riboflavin, pyridoxine (B6 group), niacin, calcium pantothenate, biotin, folic acid, betaine, amides, acetamide, lactamide, N-sugar color, pyrollidone carboxylic acid (PCA), allantoin, allantoic acid, aspartic acid, asparagine, asparagine, glutamic acid, glutamine, glutamine, α-alanine, valine, γ-aminobutyric acid, threonine, isoleucine, glycine, leucine, lysine, serine, arginine, phenylalanine, tyrosine, histidine, hydroxyproline, proline, methionine, tryptophan, uridine, adenine, pesticides, herbicides, aldrin, dieldrin, chlordane, trehalose, acetaldehyde, acetals, 3-methyl-1-butanol, 2-methyl-1-propanol, 2-propanol, 1-propanol, 1-butanol, 2-methylbutanol, sulfite waste liquor, fusel alcohols, n-pentanol, n-hexanol, n-heptanol, higher straight-chain aldehydes, pentanal, hexanal, heptanal, octanal, aromatic alcohols, phenol derivatives, silica, maillard, caramel color, organic acids, aromatic acids, polypeptides, nucleic acids, fructose, iso-maltose, sorbitol, erythritol, glycerol, lactobacillus, and combinations thereof.

69. The fatty ester composition of claim 68, further comprising an additional impurity comprising a starch.

70. The fatty ester composition of claim 69, wherein the starch is derived from a plant selected from the group consisting of barley, corn, wheat, potato, and rice.

71. The fatty ester composition of claim 60, wherein the fatty ester has a fraction of modern carbon of about 1.003 to about 1.5.

72. The fatty ester composition of claim 60, wherein the fatty ester composition comprises a mixture of fatty esters selected from the group consisting of: C12:0, C12:1, C14:0, C14:1, C16:0, C16:1, C18:0, and C18:1, wherein at least 60% by volume of the fatty esters are C16, C18, or some combination thereof.

73. The fatty ester composition of claim 60, wherein the fatty ester has the following formula:
BCOOA
wherein the B side of the fatty ester is a carbon chain comprising at least 6 carbons and the A side is an aliphatic group that comprises at least one carbon.

74. The fatty ester composition of claim 73, wherein the B side of the fatty ester is a polyunsaturated carbon chain.

75. The fatty ester composition of claim 73, wherein the B side of the fatty ester is a monounsaturated carbon chain.

76. The fatty ester composition of claim 73, wherein the B side of the fatty ester is a saturated carbon chain.

77. The fatty ester composition of claim 73, wherein the aliphatic group of the A side of the fatty ester has a number of carbon atoms selected from the group consisting of: 1, 2, 3, 4, 5, 6, 7, 8, 9, 10, 11, 12, 13, 14, 15, 16, 17, 18, 19, and 20.

78. The fatty ester composition of claim 73, wherein the B side of the fatty ester comprises a carbon chain having a number of carbon atoms selected from the group consisting of: 5, 6, 7, 8, 9, 10, 11, 12, 13, 14, 15, 16, 17, 18, 19, 20, 21, 22, 23, 24, 25, 26, 27, 28, 29, and 30.

79. The fatty ester composition of claim 78, wherein the number of carbon atoms is selected from the group consisting of 14, 15, and 16.

80. The fatty ester composition of claim 60, wherein the fatty ester has a δ¹³ of from about −10.9 to about −15.4.

81. The fatty ester composition of claim 60, wherein the fatty ester has a δ¹³ of about −28 or greater.

82. The fatty ester composition of claim 81, wherein the fatty ester has a δ¹³ of about −18 or greater.

83. The fatty ester composition of claim 60, wherein the fatty ester has a δ¹³ of about −27 to about −24.

84. The fatty ester composition of claim 83, wherein the fatty ester has a δ¹³ of about −16 to about −10.

85. The fatty ester composition of claim 60, wherein the fatty ester has a f_{M} ¹⁴C of at least about 1.

86. The fatty ester composition of claim 85, wherein the fatty ester has a f_{M} ¹⁴C of at least about 1.01.

87. The fatty ester composition of claim 60, wherein the fatty ester has a f_{M} ¹⁴C of about 1 to about 1.5.

88. The fatty ester composition of claim 87, wherein the fatty ester has a f_{M} ¹⁴C of about 1.04 to about 1.18.

89. The fatty ester composition of claim 88, wherein the fatty ester has a f_{M} ¹⁴C of about 1.111 to about 1.124.

90. A biofuel comprising a fatty ester, wherein the fatty ester is produced according to the method of claim 1.

91. The biofuel of claim 90, wherein the fatty ester has a δ¹³ of from about −10.9 to about −15.4.

92. The biofuel of claim 90, wherein the fatty ester has a f_{M} ¹⁴C of about 1.003 to about 1.5.

93. A biofuel comprising a fatty ester, wherein the fatty ester is produced according to the method of claim 26.

94. The biofuel of claim 93, wherein the fatty ester has a δ¹³ of from about −10.9 to about −15.4.

95. The biofuel of claim 94, wherein the fatty ester has a f_{M} ¹⁴C of about 1.003 to about 1.5.
